(19) United States
(12) Patent Application Publication
(10) Pub. No.: US 2026/0241183 A1
(43) Pub. Date: Aug. 20, 2026

(54) DOUBLE SEQUENTIAL EXTERNAL DEFIBRILLATION AND VECTOR CHANGE

(71) Applicant: ZOLL Medical Corporation, Chelmsford, MA (US)

(72) Inventors: Gideon D.H. Butler, Portsmouth, NH (US); Paolo Giacometti, North Grafton, MA (US); Timothy F. Stever, Lowell, MA (US); Hana Khalil Ritscher, Burlington, MA (US); Dan E. Goldman, Shrewsbury, MA (US); Joshua W. Lampe, Groton, MA (US); Gary A. Freeman, Waltham, MA (US)

(73) Assignee: ZOLL Medical Corporation, Chelmsford, MA (US)

(21) Appl. No.: 19/164,435

(22) PCT Filed: Mar. 14, 2024
(86) PCT No.: PCT/US24/19904

§ 371 Date: Sep. 11, 2025

Related U.S. Application Data
(60) Provisional application No. 63/490,599, filed on Mar. 16, 2023.

(51) Int. Cl. A61N 1/39

(52) U.S. Cl. CPC A61N 1/3904; A61N 1/3918; A61N 1/3975

(57) ABSTRACT

Dual sequential external defibrillation (DSED) and defibrillation with vector change system, devices and methods are described. An example system includes two defibrillators and a defibrillation accessory device. The defibrillators and two pairs of electrodes may connect with the accessory device, and electrical current for defibrillation shocks may pass through the defibrillation accessory device. In DSED and defibrillation with vector change, two defibrillation shocks may be delivered, where the second shock is delivered along a different vector line than the first shock. In some examples, the defibrillation accessory device may be used in alternating patient transthoracic impedance monitoring and measurement, and associated impedance values may be provided to each of the defibrillators for use in connection with determination of one or more parameters associated with defibrillation shocks.

### BACKGROUND

Electrotherapies, which can include delivery of electrical energy or current to a patient, may be used, for example, in connection with treatments relating to organs such as the human heart. Normally, electro-chemical activity within a human heart causes the organ's muscle fibers to contract and relax in a synchronized manner. This synchronized action of the heart's musculature results in the effective pumping of blood from the ventricles throughout the body, including to the vital organs. Certain conditions can disrupt this normal electrochemical activity, synchronized action and effective pumping of the heart. These conditions include cardiac arrhythmias, which can cause the heart to beat irregularly or abnormally. Electrotherapies, including defibrillation and pacing, for example, can be useful or lifesaving in connection with conditions such as cardiac arrhythmias.

In particular, one of the most deadly cardiac arrhythmias is ventricular fibrillation, during which abnormal electrical activity within the heart causes the individual muscle fibers to contract in an unsynchronized and chaotic way. As a result of this loss of synchronization, the heart can rapidly lose its ability to effectively pump blood.

Defibrillation is a type of electrotherapy that may be used to treat conditions including ventricular fibrillation. A defibrillator may produce a large shock for delivery to a patient that disrupts the chaotic electrical activity of the heart associated with ventricular fibrillation, which can allow the heart's electro-chemical system to re-synchronize itself. Once organized electrical activity is restored, synchronized muscle contractions often follow, leading to the restoration of effective cardiac pumping.

### SUMMARY

One example provides a system for double defibrillation, comprising: a first defibrillator, comprising a first accessory device connection port, configured to deliver a first defibrillation shock to a patient; a second defibrillator, comprising a second accessory device connection port, configured to deliver a second defibrillation shock to the patient; and a defibrillation accessory device, separate from the first defibrillator and the second defibrillator, comprising: a first defibrillator connection port and a second defibrillator connection port, and at least one electrode port configured for connection of at least three electrodes comprising at least two electrode pairs comprising a first pair of electrodes defining a first shock vector path and a second pair of electrodes defining a second shock vector path, wherein the first shock vector path is different than the second shock vector path, and wherein delivery of the second defibrillation shock occurs after a shock delay period following delivery of the first defibrillation shock, wherein the first defibrillator is configured to deliver the first defibrillation shock to the patient via the first pair of electrodes along the first shock vector path, and wherein the second defibrillator is configured to deliver the second defibrillation shock to the patient via the second pair of electrodes along the second shock vector path, and wherein electrical current for the first defibrillation shock passes through the defibrillation accessory device before reaching the patient, and wherein electrical current for the second defibrillation shock passes through the defibrillation accessory device before reaching the patient.

In some examples, the shock delay period is from initiation of the delivery of the first shock to initiation of the delivery of the second shock. In some examples, a time period of the delivery of the second shock partially overlaps with a time period of the delivery of the first shock. In some examples, a time period of the delivery of the second shock has no overlap with a time period of the delivery of the first shock. In some examples, delivery of the second shock is initiated after a period of time following completion of the delivery of the first shock.

In some examples, the first defibrillator and the defibrillation accessory device are configured for cable connection via the first accessory device connection port and the first defibrillator connection port, and wherein the second defibrillator and the defibrillation accessory device are configured for cable connection via the second accessory device connection port and the second defibrillator connection port, and the first pair of electrodes defines the first shock vector path between a first electrode of the first pair of electrodes and a second electrode of the first pair of electrodes, wherein the second pair of electrodes defines the second shock vector path between a first electrode of the second pair of electrodes and a second electrode of the second pair of electrodes.

In some examples, the shock delay period is between 0-300 milliseconds, between 0.1-100 milliseconds, or between 0.01-50 milliseconds. In some examples, the defibrillation accessory device is configured to trigger the delivery of the second defibrillation shock to occur after the shock delay period. In some examples, triggering the delivery of the second defibrillation shock to occur after the shock delay period following the delivery of the first defibrillation shock comprises triggering the delivery of the second defibrillation shock such that a start of a time period of the second defibrillation shock occurs after the shock delay period following a start of a time period of the first defibrillation shock. In some examples, the triggering comprises at least one current sensing circuit component configured for use in detection of electrical current of the first defibrillation shock. In some examples, the at least one current sensing circuit component comprises a transformer.

In some examples, the defibrillation accessory device is configured to: determine a first patient transthoracic impedance value associated with determining at least one electrical parameter for the first defibrillation shock, comprising sending electrical current along the first shock vector path and through the patient, and determine a second patient transthoracic impedance value associated with determining at least one electrical parameter for the second defibrillation shock, comprising sending electrical current along the second shock vector path and through the patient, wherein determining the first patient transthoracic impedance value and the second patient transthoracic impedance value comprises alternating between (1) the sending of electrical current along the first shock vector path and through the patient and (2) the sending of electrical current along the second shock vector path and through the patient, send first electrical signaling associated with the first patient transthoracic impedance value to the first defibrillator for use in determining the at least one electrical parameter for the first defibrillation shock, and send second electrical signaling associated with the second patient transthoracic impedance value to the second defibrillator for use in determining the at least one electrical parameter for the second defibrillation shock.

In some examples, the at least one electrical parameter comprises voltage. In some examples, the defibrillation accessory device is configured to electrically prevent patient transthoracic impedance monitoring by the first defibrillator and the second defibrillator. In some examples, electrically preventing patient transthoracic impedance monitoring by the first defibrillator and the second defibrillator comprises electrically blocking high frequency patient transthoracic impedance monitoring. In some examples, electrically preventing the patient transthoracic impedance monitoring by the first defibrillator and the second defibrillator comprises electrically blocking the high frequency patient transthoracic impedance monitoring while permitting low frequency patient electrocardiogram (ECG) monitoring by at least one of: the first defibrillator and the second defibrillator. In some examples, electrically preventing the patient transthoracic impedance monitoring by the first defibrillator and the second defibrillator comprises electrically blocking high frequency patient transthoracic impedance monitoring and electrically blocking low frequency patient ECG monitoring by at least one of the first defibrillator and the second defibrillator.

In some examples, electrically preventing the patient transthoracic impedance monitoring by the first defibrillator comprises use of at least one first gas discharge tube, and wherein electrically preventing the patient transthoracic impedance monitoring by the second defibrillator comprises use of at least one second gas discharge tube. In some examples, the defibrillation accessory device comprises an electrical circuit to allow patient ECG monitoring by the first defibrillator, wherein electrical current along the electrical circuit does not pass through the at least one first gas discharge tube or the at least one second gas discharge tube. In some examples, causing the sending of the first electrical signaling comprises: determining a first resistance value associated with the determined first patient transthoracic impedance value; and
selecting a first electrical circuit configuration, for a first programmable resistor circuit, associated with the determined first resistance value; and wherein causing the sending of the second electrical signaling comprises: determining a second resistance value associated with the determined second patient transthoracic impedance value; and selecting a second electrical circuit configuration, for a second programmable resistor circuit, to reflect the determined second resistance value.

In some examples, selecting the first electrical circuit configuration comprises determining a configuration of at least one switch of the first programmable resistor circuit as open or closed, and wherein selecting the second electrical circuit configuration comprises determining a configuration of at least one switch of the second programmable resistor circuit as open or closed. In some examples, the first resistance value is selected from a set of available resistance values, wherein the first resistance value is closest, among the set of available resistance values, to the determined first patient transthoracic impedance value, and wherein the second resistance value is selected from the set of available resistance values, wherein the second resistance value is closest, among the set of available resistance values, to the determined second patient transthoracic impedance value. In some examples, the first resistance value is determined so as to be approximately equal to the first patient transthoracic impedance value, and wherein the second resistance value is determined so as to be approximately equal to the second patient transthoracic impedance value. In some examples, the first programmable resistor circuit and the second programmable resistor circuit are PWM based.

In some examples, the system comprises: a first voltage regulator circuit, electrically connected between the first defibrillator and the first programmable resistor circuit, configured to protect at least the first programmable resistor circuit from over-voltage during delivery of the first defibrillation shock; and a second voltage regulator circuit, electrically connected between the second defibrillator and the second programmable resistor circuit, configured to protect at least the second programmable resistor circuit from over-voltage during delivery of the second defibrillation shock. In some examples, each of the first voltage regulator circuit and the second voltage regulator circuit comprises at least one cascode circuit. In some examples, the first voltage regulator comprises: a first cascode circuit configured to protect at least the first programmable resistor circuit from overvoltage during a positive phase of the first defibrillation shock, and a second cascode circuit configured to protect at least the first programmable resistor circuit from overvoltage during a negative phase of the first defibrillation shock, and the second voltage regulator comprises: a third cascode circuit configured to protect at least the second programmable resistor circuit from overvoltage during a positive phase of the second defibrillation shock, and a fourth cascode circuit configured to protect at least the second programmable resistor circuit from overvoltage during a negative phase of the second defibrillation shock.

In some examples, the first cascode circuit comprises a first high voltage transistor and a first low voltage transistor, the second cascode circuit comprises a second high voltage transistor and a second low voltage transistor, the third cascode circuit comprises a third high voltage transistor and a third low voltage transistor, and the fourth cascode circuit comprises a fourth high voltage transistor and a fourth low voltage transistor. In some examples, the first low voltage transistor is electrically connected between the first high voltage transistor and the first programmable resistor circuit, the second low voltage transistor is electrically connected between the second high voltage transistor and the first programmable resistor circuit, the third low voltage transistor is electrically connected between the third high voltage transistor and the second programmable resistor circuit, and the fourth low voltage transistor is electrically connected between the fourth high voltage transistor and the second programmable resistor circuit.

In some examples, the first defibrillator comprises: an energy storage capacitor for providing electrotherapeutic current to the patient, wherein the first defibrillation shock comprises the electrotherapeutic current; a therapeutic current control network, electrically coupled to the energy storage capacitor, for controlling the electrotherapeutic current to be delivered to the patient, the therapeutic current control network comprising a resonant electrical circuit and at least one current control switch, wherein energy provided by the energy storage capacitor flows through the resonant electrical circuit; and a controller electrically coupled to the energy storage capacitor and the therapeutic current control network, wherein the controller is configured to, in connection with the energy provided by the energy storage capacitor that flows through the resonant electrical circuit, control operation of the at least one current control switch of the therapeutic current control network in delivering an electrotherapeutic waveform to the patient to correspond with a specified waveform.

In some examples, the first defibrillator comprises: at least one sensor configured to sense at least one electrical parameter from which current flow to the patient can be determined or estimated, wherein the controller is configured to: process a signal associated with the sensed at least one electrical parameter; compare the processed signal with a second signal associated with the specified waveform; and control operation of the at least one current control switch of the therapeutic current control network in adjusting delivery of the electrotherapeutic waveform to the patient to correspond with the specified waveform.

In some examples, the first defibrillator and the second defibrillator comprises: a battery; and a bidirectional charging control network, comprising: a first circuit connected with the battery, a second circuit connected with the energy storage capacitor, and a transformer, connected with the first circuit and the second circuit, for use in storing first energy from the battery and in transferring at least a portion of the stored first energy to the energy storage capacitor, and for use in storing second energy from the energy storage capacitor and in transferring at least a portion of the stored second energy to the battery.

In some examples, the first defibrillator comprises: an energy storage capacitor for providing electrotherapeutic current to the patient, wherein the first defibrillation shock comprises the electrotherapeutic current; a therapeutic current control network, electrically coupled to the energy storage capacitor, for controlling the electrotherapeutic current to be delivered to the patient, the therapeutic current control network comprising a resonant electrical circuit in and at least one current control switch, wherein energy provided by the energy storage capacitor flows through the resonant electrical circuit, the therapeutic current control network being configured to be operable in an operation mode of two modes of operation, the two modes comprising: a first mode in which the resonant electrical circuit is connected with the patient in a parallel configuration, and a second mode in which the resonant electrical circuit is connected with the patient in a series configuration; and a controller electrically coupled to the energy storage capacitor and the therapeutic current control network, wherein the controller is configured to, in connection with the energy provided by the energy storage capacitor that flows through the resonant electrical circuit,
control operation of the at least one current control switch of the therapeutic current control network in delivering an electrotherapeutic waveform to the patient to correspond with a specified waveform.

In some examples, the operation mode is selected based at least in part on maximizing a value of a Q factor, wherein the value of the Q factor represents a measure of peak energy relative to dissipated energy with regard to the resonant electrical circuit, wherein the value of the Q factor is affected by the operation mode and a patient impedance. In some examples, at least one of: the first defibrillation shock and the second defibrillation shock, comprises a biphasic shock. In some examples, the first defibrillation shock comprises a positive phase shock vector and a negative phase shock vector, wherein a positive phase of the biphasic shock is delivered according to the positive phase shock vector, and wherein a negative phase of the biphasic shock is delivered according to the negative phase shock vector, and wherein the positive phase shock vector and the negative phase shock vector are in opposite directions along the first shock vector path. In some examples, at least one of the first defibrillation shock and the second defibrillation shock, comprises a biphasic rectilinear current waveform.

In some examples, at least one of: the first defibrillation shock and the second defibrillation shock, comprises a truncated exponential current waveform. In some examples, at least one of the first defibrillation shock and the second defibrillation shock, comprises a monophasic shock. In some examples, at least one of the first shock vector path and the second shock vector path defines an anterior-lateral path through a torso of the patient. In some examples, at least one of the first shock vector path and the second shock vector path defines an anterior-posterior path through a torso of the patient. In some examples, the first shock vector path defines an anterior-lateral path through a torso of the patient and the second shock vector path defines an anterior-posterior path through the torso of the patient. In some examples, the first shock vector path defines an anterior-posterior path through a torso of the patient and the second shock vector path defines an anterior-lateral path through the torso of the patient.

In some examples, the first pair of electrodes and the second pair of electrodes have an electrode in common. In some examples, the at least three electrodes cable connected to the defibrillation accessory device via the at least one electrode port. In some examples, the system comprises a multielectrode pad configured to comprise a plurality of constituent electrodes, wherein each of the plurality constituent electrodes is for placement on a different area of the patient's torso, wherein at least one of the at least three electrodes is a constituent electrode of the plurality of constituent electrodes. In some examples, the first defibrillator comprises a first controller, wherein the second defibrillator comprises a second controller, and wherein accessory device comprises a third controller. In some examples, the first defibrillator is configured to deliver the first defibrillation shock upon a corresponding selection by a care provider. In some examples, the selection comprises pressing of a button that causes delivery of the first defibrillation shock. In some examples, the first defibrillator is configured to deliver the first defibrillation shock at a preset first shock delivery time, wherein the preset first shock delivery time is set prior to delivery of the first defibrillation shock.

In some examples, the first defibrillator is a master defibrillator configured to deliver the first defibrillation shock at a first time that is independent of a second time of delivery of the second defibrillation shock, and the second defibrillator is a slave defibrillator, wherein the second time of delivery of the second defibrillation shock is dependent upon the first time of delivery of the first defibrillation shock. In some examples, the shock delay period is user configurable. In some examples, the shock delay period is not user configurable.

One example provides a system for double defibrillation, comprising: at least one defibrillator configured to: deliver a first defibrillation shock to a patient according to at least one first shock vector along a first shock vector path, the first shock vector path being defined by a first pair of electrodes, and deliver a second defibrillation shock to the patient according to at least one second shock vector along a second shock vector path, the second shock vector path being defined by a second pair of electrodes, wherein the first shock vector path is different than the second shock vector path, and wherein delivery of the second defibrillation shock occurs after a shock delay period following delivery of the first defibrillation shock, wherein the system is configured to: determine a first patient transthoracic impedance value associated with determining at least one electrical parameter for the first defibrillation shock, comprising sending electrical current along the first shock vector path and through the patient, and determine a second patient transthoracic impedance value associated with determining at least one electrical parameter for the second defibrillation shock, comprising sending electrical current along the second shock vector path and through the patient, wherein determining the first patient transthoracic impedance value and the second patient transthoracic impedance value comprises alternating between (1) the sending of electrical current along the first shock vector path and through the patient and (2) the sending of electrical current along the second shock vector path and through the patient.

In some examples, alternating between (1) the sending of electrical current along the first shock vector path and through the patient and (2) the sending of electrical current along the second shock vector path and through the patient comprises continuously sending electrical current, wherein continuously sending electrical current comprises alternating over time between (1) first periods of time of the sending of electrical current along the first shock vector path and through the patient and (2) second periods of time of the sending of electrical current along the second shock vector path and through the patient, wherein, at any particular time, current is either being sent along the first shock vector path and through the patient or along the second shock vector path and through the patient.

In some examples, each of the first periods of time and the second periods of time are between 0.01 and 5 seconds, between 0.1 and 3 seconds, between 0.1 and 1 second, or between 0.3 and 0.7 second. In some examples, each of the first periods of time and the second periods of time are approximately 0.5 second. In some examples, the shock delay period is between 0-300 milliseconds, between 0.1-100 milliseconds, or between 0.01-50 milliseconds. In some examples, the at least one defibrillator comprises a first defibrillator configured to deliver the first defibrillation shock and a second defibrillator configured to deliver the second defibrillation shock. In some examples, at least a first defibrillator of the at least one defibrillator is configured to monitor for the patient transthoracic impedance.

In some examples, the at least one defibrillator comprises at least one accessory device connection port, and comprising a defibrillation accessory device, separate from the at least one defibrillator, the defibrillation accessory device comprising: at least one defibrillator connection port, wherein at least one defibrillator and the defibrillation accessory device are configured for cable connection via the at least one accessory device connection port and the at least one defibrillator connection port, wherein electrical current for the first defibrillation shock passes through the defibrillation accessory device before reaching the patient, and wherein electrical current for the second defibrillation shock passes through the defibrillation accessory device before reaching the patient. In some examples, at least one of the first defibrillation shock and the second defibrillation shock comprises a biphasic shock.

In some examples, the at least one first shock vector comprises two shock vectors include a positive phase shock vector and a negative phase shock vector, wherein a positive phase of the biphasic shock is delivered according to the positive phase shock vector, and wherein a negative phase of the biphasic shock is delivered according to the negative phase shock vector, and wherein the positive phase shock vector and the negative phase shock vector are in opposite directions along the first shock vector path. In some examples, at least one of: the first defibrillation shock and the second defibrillation shock, comprises a biphasic rectilinear shock. In some examples, at least one of the first defibrillation shock and the second defibrillation shock, comprises a truncated exponential shock. In some examples, at least one of the first defibrillation shock and the second defibrillation shock, comprises a monophasic shock.

In some examples, at least one of the first shock vector path and the second shock vector path defines an anterior-lateral path through a torso of the patient. In some examples, at least one of the first shock vector path and the second shock vector path defines an anterior-posterior path through a torso of the patient. In some examples, the first shock vector path defines an anterior-lateral path through a torso of the patient and the second shock vector path defines an anterior-posterior path through the torso of the patient. In some examples, the first shock vector path defines an anterior-posterior path through a torso of the patient and the second shock vector path defines an anterior-lateral path through the torso of the patient. In some examples, three electrodes comprise the first pair of electrodes and the second pair of electrodes, and wherein the first pair of electrodes and the second pair of electrodes have a first electrode, of the three electrodes, in common. In some examples, the at least three electrodes cable connected to the defibrillation accessory device via the at least one electrode port.

One example provides a system for double defibrillation, comprising: a first defibrillator configured to deliver a first defibrillation shock to a patient according to at least one first shock vector along a first shock vector path, the first shock vector path being defined by a first pair of electrodes; a second defibrillator configured to deliver a second defibrillation shock to a patient according to at least one second shock vector along a second shock vector path, the second shock vector path being defined by a second pair of electrodes, the second shock vector path being different than the first shock vector path, wherein delivery of the second defibrillation shock occurs after a shock delay period following delivery of the first defibrillation shock; and a defibrillation accessory device, separate from the first defibrillator and the second defibrillator, configured for cable connection to the first defibrillator and the second defibrillator, configured to: electrically prevent patient transthoracic impedance monitoring by the first defibrillator and the second defibrillator, determine a first patient transthoracic impedance value associated with determining at least one electrical parameter for the first defibrillation shock, comprising sending electrical current along the first shock vector path and through the patient, and determine a second patient transthoracic impedance value associated with determining at least one electrical parameter for the second defibrillation shock, comprising sending electrical current along the second shock vector path and through the patient.

In some examples, electrical current for the first defibrillation shock passes through the defibrillation accessory device before reaching the patient, and wherein electrical current for the second defibrillation shock passes through the defibrillation accessory device before reaching the patient, wherein determining the first patient transthoracic impedance value and the second patient transthoracic impedance value comprises alternating between (1) the sending of electrical current along the first shock vector path and through the patient and (2) the sending of electrical current along the second shock vector path and through the patient, and wherein the defibrillation accessory device is configured to: cause sending of first electrical signaling associated with the first patient transthoracic impedance value to the first defibrillator for use in determining the at least one electrical parameter for the first defibrillation shock, and cause sending of second electrical signaling associated with the second patient transthoracic impedance value to the second defibrillator for use in determining the at least one electrical parameter for the second defibrillation shock.

In some examples, the defibrillation accessory device comprises: a first measurement circuit configured to measure the first patient transthoracic impedance value: a second measurement circuit configured to measure the first patient transthoracic impedance value; a first isolator circuit configured to be connected with the first measurement circuit; and a second isolator circuit configured to be connected with the second measurement circuit. In some examples, the first isolator circuit comprises a first capacitive isolator circuit. In some examples, the second isolator circuit comprises a second capacitive isolator circuit. In some examples, the shock delay period is between 0-300 milliseconds, between 0.1-100 milliseconds, or between 0.01-50 milliseconds.

In some examples, the defibrillation accessory device is configured to trigger the delivery of the second defibrillation shock to occur after the shock delay period. In some examples, triggering the delivery of the second defibrillation shock to occur after the shock delay period following the delivery of the first defibrillation shock comprises triggering the delivery of the second defibrillation shock such that a start of a time period of the second defibrillation shock occurs after the shock delay period following a start of a time period of the first defibrillation shock. In some examples, the triggering comprises at least one current sensing circuit component configured for use in detection of electrical current of the first defibrillation shock. In some examples, the at least one current sensing circuit component comprises a transformer. In some examples, the defibrillation accessory device comprises: a triggering circuit configured to trigger the delivery of the second defibrillation shock; and an electrical isolator circuit coupled between the current sensing circuit and the triggering circuit. In some examples, electrical isolator circuit comprises at least one transformer.

In some examples, electrically preventing patient transthoracic impedance monitoring by the first defibrillator and the second defibrillator comprises electrically blocking high frequency patient transthoracic impedance monitoring. In some examples, electrically preventing the patient transthoracic impedance monitoring by the first defibrillator and the second defibrillator comprises electrically blocking the high frequency patient transthoracic impedance monitoring while permitting low frequency patient electrocardiogram (ECG) monitoring by at least one of: the first defibrillator and the second defibrillator. In some examples, electrically preventing the patient transthoracic impedance monitoring by the first defibrillator and the second defibrillator comprises electrically blocking high frequency patient transthoracic impedance monitoring and electrically blocking low frequency patient ECG monitoring by at least one of the first defibrillator and the second defibrillator. In some examples, electrically preventing the patient transthoracic impedance monitoring by the first defibrillator comprises use of at least one first gas discharge tube, and wherein electrically preventing the patient transthoracic impedance monitoring by the second defibrillator comprises use of at least one second gas discharge tube. In some examples, the defibrillation accessory device comprises an electrical circuit to allow patient ECG monitoring by the first defibrillator, wherein electrical current along the electrical circuit does not pass through the at least one first gas discharge tube. In some examples, the defibrillation accessory device comprises an electrical circuit to allow patient ECG monitoring by the second defibrillator, wherein electrical current along the electrical circuit does not pass through the at least one second gas discharge tube.

In some examples, causing the sending of the first electrical signaling comprises: determining a first resistance value associated with the determined first patient transthoracic impedance value; and selecting a first electrical circuit configuration, for a first programmable resistor circuit, associated with the determined first resistance value; and wherein causing the sending of the second electrical signaling comprises: determining a second resistance value associated with the determined second patient transthoracic impedance value; and selecting a second electrical circuit configuration, for a second programmable resistor circuit, to reflect the determined second resistance value. In some examples, selecting the first electrical circuit configuration comprises determining a configuration of at least one switch of the first programmable resistor circuit as open or closed, and wherein selecting the second electrical circuit configuration comprises determining a configuration of at least one switch of the second programmable resistor circuit as open or closed.

In some examples, the first resistance value is selected from a set of available resistance values, wherein the first resistance value is closest, among the set of available resistance values, to the determined first patient transthoracic impedance value, and wherein the second resistance value is selected from the set of available resistance values, wherein the second resistance value is closest, among the set of available resistance values, to the determined second patient transthoracic impedance value. In some examples, the first resistance value is determined so as to be approximately equal to the first patient transthoracic impedance value, and wherein the second resistance value is determined so as to be approximately equal to the second patient transthoracic impedance value. In some examples, the first programmable resistor is PWM based.

In some examples, the system comprises: a first voltage regulator circuit, electrically connected between the first defibrillator and the first programmable resistor circuit, configured to protect at least the first programmable resistor circuit from over-voltage during delivery of the first defibrillation shock; and a second voltage regulator circuit, electrically connected between the second defibrillator and the second programmable resistor circuit, configured to protect at least the second programmable resistor circuit from over-voltage during delivery of the second defibrillation shock. In some examples, each of the first voltage regulator circuit and the second voltage regulator circuit comprises at least one cascode circuit. In some examples, the first voltage regulator comprises: a first cascode circuit configured to protect at least the first programmable resistor circuit from overvoltage during a positive phase of the first defibrillation shock, and a second cascode circuit configured to protect at least the first programmable resistor circuit from overvoltage during a negative phase of the first defibrillation shock, and the second voltage regulator comprises: a third cascode circuit configured to protect at least the second programmable resistor circuit from overvoltage during a positive phase of the second defibrillation shock, and a fourth cascode circuit configured to protect at least the second programmable resistor circuit from overvoltage during a negative phase of the second defibrillation shock.

In some examples, the first cascode circuit comprises a first high voltage transistor and a first low voltage transistor, the second cascode circuit comprises a second high voltage transistor and a second low voltage transistor, the third cascode circuit comprises a third high voltage transistor and a third low voltage transistor, and the fourth cascode circuit comprises a fourth high voltage transistor and a fourth low voltage transistor. In some examples, the first low voltage transistor is electrically connected between the first high voltage transistor and the first programmable resistor circuit, the second low voltage transistor is electrically connected between the second high voltage transistor and the first programmable resistor circuit, the third low voltage transistor is electrically connected between the third high voltage transistor and the second programmable resistor circuit, and the fourth low voltage transistor is electrically connected between the fourth high voltage transistor and the second programmable resistor circuit. In some examples, the defibrillation accessory device comprises at least one electrical circuit configured for electrically preventing the transthoracic impedance monitoring, determining the first patient transthoracic impedance, and determining the second patient transthoracic impedance.

One example provides a system for double defibrillation, comprising: a first defibrillator configured to deliver a first defibrillation shock to a patient according to at least one first shock vector along a first shock vector path, the first shock vector path being defined by a first pair of electrodes; a second defibrillator configured to deliver a second defibrillation shock to a patient according to at least one first shock vector along a second shock vector path, the second shock vector path being defined by a second pair of electrodes, the second shock vector path being different than the first shock vector path, wherein delivery of the second defibrillation shock occurs after a shock delay period following delivery of the first defibrillation shock; and a defibrillation accessory device, separate from the first defibrillator and the second defibrillator, configured for cable connection to the first defibrillator and the second defibrillator, wherein electrical current for the first defibrillation shock passes through the defibrillation accessory device before reaching the patient, and wherein electrical current for the second defibrillation shock passes through the defibrillation accessory device before reaching the patient, the defibrillation accessory device being configured to trigger the delivery of the second defibrillation shock to occur after the shock delay period.

In some examples, triggering the delivery of the second defibrillation shock to occur after the shock delay period following the delivery of the first defibrillation shock comprises triggering the delivery of the second defibrillation shock such that a start of a time period of the second defibrillation shock occurs after the shock delay period following a start of a time period of the first defibrillation shock. In some examples, the triggering comprises at least one current sensing circuit component configured for use in detection of electrical current of the first defibrillation shock. In some examples, the at least one current sensing circuit component comprises a transformer. In some examples, the shock delay period is between 0-300 milliseconds, between 0.1-100 milliseconds, or between 0.01-50 milliseconds.

One example provides a system for double defibrillation, comprising: at least one defibrillator configured to: deliver a first defibrillation shock to a patient according to at least one first shock vector along a first shock vector path, the first shock vector path being defined by a first pair of electrodes, and deliver a second defibrillation shock to a patient according to at least one second shock vector along a second shock vector path, the second shock vector path being defined by a second pair of electrodes, wherein the second shock vector path is different than the first shock vector path, and wherein delivery of the second defibrillation shock occurs after a shock delay period following delivery of the first defibrillation shock, wherein the system is configured to: based at least in part on the time of initiation of the first defibrillation shock and the delay, trigger initiation of the second defibrillation shock upon the expiration of the delay, determine a first patient transthoracic impedance value associated with determining at least one electrical parameter for the first defibrillation shock, comprising sending electrical current along the first shock vector path and through the patient, and determine a second patient transthoracic impedance value associated with determining at least one electrical parameter for the second defibrillation shock, comprising sending electrical current along the second shock vector path and through the patient. In some examples, the shock delay period is between 0-300 milliseconds, between 0.1-100 milliseconds, or between 0.01-50 milliseconds.

One example provides a method for double defibrillation, comprising: determining a first patient transthoracic impedance value to be used in determining at least one electrical parameter for a first defibrillation shock, comprising sending electrical current along a first shock vector path and through a patient, the first shock vector path being defined by a first pair of electrodes; determining a second patient transthoracic impedance value to be used in determining at least one electrical parameter for the second defibrillation shock, comprising sending electrical current along a second shock vector path and through the patient, the second shock vector path being defined by a second pair of electrodes, wherein the second shock vector path is different than the first shock vector path, wherein determining the first patient transthoracic impedance value and the second patient transthoracic impedance value comprises alternating between (1) the sending of electrical current along the first shock vector path and through the patient and (2) the sending of electrical current along the second shock vector path and through the patient; the first defibrillator delivering the first defibrillation shock to the patient according to at least one first shock vector along the first shock vector path; and the second defibrillator delivering the second defibrillation shock to the patient according to at least one second shock vector along the second shock vector path, comprising initiating the second defibrillation shock upon expiration of a delay of a shock delay period following a time of initiation of the first defibrillation shock, wherein the shock delay period is between 0-100 milliseconds.

In some examples, the method comprises: causing sending of first electrical signaling associated with the first patient transthoracic impedance value to a first defibrillator for use in determining the at least one electrical parameter for the first defibrillation shock, and causing sending of second electrical signaling associated with the second patient transthoracic impedance value to a second defibrillator for use in determining the at least one electrical parameter for the second defibrillation shock. In some examples, the shock delay period is between 50-100 milliseconds, between 1-50 milliseconds, or between 0.01-1 millisecond. In some examples, determining the first patient transthoracic impedance value and the second patient transthoracic impedance value comprises alternating between (1) the sending of electrical current along the first shock vector path and through the patient and (2) the sending of electrical current along the second shock vector path and through the patient. In some examples, alternating between (1) the sending of electrical current along the first shock vector path and through the patient and (2) the sending of electrical current along the second shock vector path and through the patient comprises continuously sending electrical current, wherein continuously sending electrical current comprises alternating over time between (1) first periods of time of the sending of electrical current along the first shock vector path and through the patient and (2) second periods of time of the sending of electrical current along the second shock vector path and through the patient, wherein, at any particular time, current is either being sent along the first shock vector path and through the patient or along the second shock vector path and through the patient.

One example provides a system for double defibrillation, comprising: a first defibrillator configured to deliver a first defibrillation shock to a patient according to at least one first shock vector along a first shock vector path, the first shock vector path being defined by a first pair of electrodes; a second defibrillator configured to deliver a second defibrillation shock to a patient according to at least one second shock vector along a second shock vector path, the second shock vector path being defined by a second pair of electrodes, the second shock vector path being different than the first shock vector path, wherein delivery of the second defibrillation shock occurs after a shock delay period following delivery of the first defibrillation shock; and a defibrillation accessory device, separate from the first defibrillator and the second defibrillator, comprising: a first programmable resistor circuit configured for use in providing a first transthoracic impedance value to the first defibrillator, the first transthoracic impedance value being associated with determination of at least one electrical parameter for the first defibrillation shock; a second programmable resistor circuit configured for use in providing a second transthoracic impedance value to the second defibrillator, the second transthoracic impedance value being associated with determination of at least one electrical parameter for the second defibrillation shock: a first voltage regulator circuit, connected between the first defibrillator and the first programmable resistor circuit, configured to protect at least the first programmable resistor circuit from over-voltage during delivery of the first defibrillation shock; and a second voltage regulator circuit, electrically connected between the second defibrillator and the second programmable resistor circuit, configured to protect at least the second programmable resistor circuit from over-voltage during delivery of the second defibrillation shock.

In some examples, the shock delay period is between 0-300 milliseconds, between 0.1-100 milliseconds, or between 0.01-50 milliseconds. In some examples, each of the first voltage regulator circuit and the second voltage regulator circuit comprises at least one cascode circuit. In some examples, the first voltage regulator comprises: a first cascode circuit configured to protect at least the first programmable resistor circuit from overvoltage during a positive phase of the first defibrillation shock, and a second cascode circuit configured to protect at least the first programmable resistor circuit from overvoltage during a negative phase of the first defibrillation shock, and the second voltage regulator comprises: a third cascode circuit configured to protect at least the second programmable resistor circuit from overvoltage during a positive phase of the second defibrillation shock, and a fourth cascode circuit configured to protect at least the second programmable resistor circuit from overvoltage during a negative phase of the second defibrillation shock.

In some examples, the first cascode circuit comprises a first high voltage transistor and a first low voltage transistor, the second cascode circuit comprises a second high voltage transistor and a second low voltage transistor, the third cascode circuit comprises a third high voltage transistor and a third low voltage transistor, and the fourth cascode circuit comprises a fourth high voltage transistor and a fourth low voltage transistor. In some examples, the first low voltage transistor is electrically connected between the first high voltage transistor and the first programmable resistor circuit, the second low voltage transistor is electrically connected between the second high voltage transistor and the first programmable resistor circuit, the third low voltage transistor is electrically connected between the third high voltage transistor and the second programmable resistor circuit, and the fourth low voltage transistor is electrically connected between the fourth high voltage transistor and the second programmable resistor circuit. In some examples, the defibrillation accessory device comprises at least one electrical circuit configured for electrically preventing the transthoracic impedance monitoring, determining the first patient transthoracic impedance, and determining the second patient transthoracic impedance. In some examples, the defibrillation accessory device comprises: a first digital isolator circuit connected with the first programmable resistor circuit; and a second digital isolator circuit connected with the second programmable resistor circuit.

One example provides a system for double defibrillation, comprising: a first defibrillator, comprising a first accessory device connection port, configured to deliver a first defibrillation shock to a patient; a second defibrillator, comprising a second accessory device connection port, configured to deliver a second defibrillation shock to the patient; and a defibrillation accessory device, separate from the first defibrillator and the second defibrillator, comprising: a first defibrillator connection port and a second defibrillator connection port, and at least one electrode port configured for connection of at least three electrodes comprising at least two electrode pairs comprising a first pair of electrodes defining a first shock vector path and a second pair of electrodes defining a second shock vector path, wherein the first shock vector path is different than the second shock vector path, wherein the first defibrillator is configured to deliver the first defibrillation shock to the patient via the first pair of electrodes along the first shock vector path, and wherein the second defibrillator is configured to deliver the second defibrillation shock to the patient via the second pair of electrodes along the second shock vector path, and wherein electrical current for the first defibrillation shock passes through the defibrillation accessory device before reaching the patient, and wherein electrical current for the second defibrillation shock passes through the defibrillation accessory device before reaching the patient.

In some examples, the first shock is delivered simultaneously with the second shock. In some examples, the delivery of the first shock and the delivery of the second shock are initiated simultaneously. In some examples, the first defibrillator and the defibrillation accessory device are configured for cable connection via the first accessory device connection port and the first defibrillator connection port, and wherein the second defibrillator and the defibrillation accessory device are configured for cable connection via the second accessory device connection port and the second defibrillator connection port, and wherein the first pair of electrodes defines the first shock vector path between a first electrode of the first pair of electrodes and a second electrode of the first pair of electrodes, wherein the second pair of electrodes defines the second shock vector path between a first electrode of the second pair of electrodes and a second electrode of the second pair of electrodes.

In some examples, the defibrillation accessory device is configured to trigger the delivery of the second defibrillation shock simultaneously with the delivery of the first shock. In some examples, the triggering comprises at least one current sensing circuit component configured for use in detection of electrical current of the first defibrillation shock. In some examples, the at least one current sensing circuit component comprises a transformer. In some examples, the defibrillation accessory device is configured to: determine a first patient transthoracic impedance value associated with determining at least one electrical parameter for the first defibrillation shock, comprising sending electrical current along the first shock vector path and through the patient, and determine a second patient transthoracic impedance value associated with determining at least one electrical parameter for the second defibrillation shock, comprising sending electrical current along the second shock vector path and through the patient, wherein determining the first patient transthoracic impedance value and the second patient transthoracic impedance value comprises alternating between (1) the sending of electrical current along the first shock vector path and through the patient and (2) the sending of electrical current along the second shock vector path and through the patient, send first electrical signaling associated with the first patient transthoracic impedance value to the first defibrillator for use in determining the at least one electrical parameter for the first defibrillation shock, and send second electrical signaling associated with the second patient transthoracic impedance value to the second defibrillator for use in determining the at least one electrical parameter for the second defibrillation shock.

In some examples, the at least one electrical parameter comprises voltage. In some examples, the defibrillation accessory device is configured to electrically prevent patient transthoracic impedance monitoring by the first defibrillator and the second defibrillator. In some examples, electrically preventing patient transthoracic impedance monitoring by the first defibrillator and the second defibrillator comprises electrically blocking high frequency patient transthoracic impedance monitoring. In some examples, electrically preventing the patient transthoracic impedance monitoring by the first defibrillator and the second defibrillator comprises electrically blocking the high frequency patient transthoracic impedance monitoring while permitting low frequency patient electrocardiogram (ECG) monitoring by at least one of the first defibrillator and the second defibrillator. In some examples, electrically preventing the patient transthoracic impedance monitoring by the first defibrillator and the second defibrillator comprises electrically blocking high frequency patient transthoracic impedance monitoring and electrically blocking low frequency patient ECG monitoring by at least one of the first defibrillator and the second defibrillator. In some examples, electrically preventing the patient transthoracic impedance monitoring by the first defibrillator comprises use of at least one first gas discharge tube, and wherein electrically preventing the patient transthoracic impedance monitoring by the second defibrillator comprises use of at least one second gas discharge tube. In some examples, the defibrillation accessory device comprises an electrical circuit to allow patient ECG monitoring by the first defibrillator, wherein electrical current along the electrical circuit does not pass through the at least one first gas discharge tube or the at least one second gas discharge tube.

In some examples, causing the sending of the first electrical signaling comprises: determining a first resistance value associated with the determined first patient transthoracic impedance value; and selecting a first electrical circuit configuration, for a first programmable resistor circuit, associated with the determined first resistance value; and wherein causing the sending of the second electrical signaling comprises: determining a second resistance value associated with the determined second patient transthoracic impedance value; and selecting a second electrical circuit configuration, for a second programmable resistor circuit, to reflect the determined second resistance value. In some examples, selecting the first electrical circuit configuration comprises determining a configuration of at least one switch of the first programmable resistor circuit as open or closed, and wherein selecting the second electrical circuit configuration comprises determining a configuration of at least one switch of the second programmable resistor circuit as open or closed.

In some examples, the first resistance value is selected from a set of available resistance values, wherein the first resistance value is closest, among the set of available resistance values, to the determined first patient transthoracic impedance value, and wherein the second resistance value is selected from the set of available resistance values, wherein the second resistance value is closest, among the set of available resistance values, to the determined second patient transthoracic impedance value. In some examples, the first resistance value is determined so as to be approximately equal to the first patient transthoracic impedance value, and wherein the second resistance value is determined so as to be approximately equal to the second patient transthoracic impedance value. In some examples, the first programmable resistor circuit and the second programmable resistor circuit are PWM based.

In some examples, the system comprises: a first voltage regulator circuit, electrically connected between the first defibrillator and the first programmable resistor circuit, configured to protect at least the first programmable resistor circuit from over-voltage during delivery of the first defibrillation shock; and a second voltage regulator circuit, electrically connected between the second defibrillator and the second programmable resistor circuit, configured to protect at least the second programmable resistor circuit from over-voltage during delivery of the second defibrillation shock. In some examples, the first voltage regulator circuit and the second voltage regulator circuit comprises at least one cascode circuit. In some examples, the first voltage regulator comprises: a first cascode circuit configured to protect at least the first programmable resistor circuit from overvoltage during a positive phase of the first defibrillation shock, and a second cascode circuit configured to protect at least the first programmable resistor circuit from overvoltage during a negative phase of the first defibrillation shock, and the second voltage regulator comprises: a third cascode circuit configured to protect at least the second programmable resistor circuit from overvoltage during a positive phase of the second defibrillation shock, and a fourth cascode circuit configured to protect at least the second programmable resistor circuit from overvoltage during a negative phase of the second defibrillation shock.

In some examples, the first cascode circuit comprises a first high voltage transistor and a first low voltage transistor, the second cascode circuit comprises a second high voltage transistor and a second low voltage transistor, the third cascode circuit comprises a third high voltage transistor and a third low voltage transistor, and the fourth cascode circuit comprises a fourth high voltage transistor and a fourth low voltage transistor. In some examples, the first low voltage transistor is electrically connected between the first high voltage transistor and the first programmable resistor circuit, the second low voltage transistor is electrically connected between the second high voltage transistor and the first programmable resistor circuit, the third low voltage transistor is electrically connected between the third high voltage transistor and the second programmable resistor circuit, and the fourth low voltage transistor is electrically connected between the fourth high voltage transistor and the second programmable resistor circuit.

In some examples, the first defibrillator comprises: an energy storage capacitor for providing electrotherapeutic current to the patient, wherein the first defibrillation shock comprises the electrotherapeutic current; a therapeutic current control network, electrically coupled to the energy storage capacitor, for controlling the electrotherapeutic current to be delivered to the patient, the therapeutic current control network comprising a resonant electrical circuit and at least one current control switch, wherein energy provided by the energy storage capacitor flows through the resonant electrical circuit; and a controller electrically coupled to the energy storage capacitor and the therapeutic current control network, wherein the controller is configured to, in connection with the energy provided by the energy storage capacitor that flows through the resonant electrical circuit, control operation of the at least one current control switch of the therapeutic current control network in delivering an electrotherapeutic waveform to the patient to correspond with a specified waveform.

In some examples, the first defibrillator comprises: at least one sensor configured to sense at least one electrical parameter from which current flow to the patient can be determined or estimated, wherein the controller is configured to: process a signal associated with the sensed at least one electrical parameter; compare the processed signal with a second signal associated with the specified waveform; and control operation of the at least one current control switch of the therapeutic current control network in adjusting delivery of the electrotherapeutic waveform to the patient to correspond with the specified waveform. In some examples, the first defibrillator and the second defibrillator comprises: a battery; and a bidirectional charging control network, comprising: a first circuit connected with the battery, a second circuit connected with the energy storage capacitor, and a transformer, connected with the first circuit and the second circuit, for use in storing first energy from the battery and in transferring at least a portion of the stored first energy to the energy storage capacitor, and for use in storing second energy from the energy storage capacitor and in transferring at least a portion of the stored second energy to the battery.

In some examples, the first defibrillator comprises: an energy storage capacitor for providing electrotherapeutic current to the patient, wherein the first defibrillation shock comprises the electrotherapeutic current; a therapeutic current control network, electrically coupled to the energy storage capacitor, for controlling the electrotherapeutic current to be delivered to the patient, the therapeutic current control network comprising a resonant electrical circuit in and at least one current control switch, wherein energy provided by the energy storage capacitor flows through the resonant electrical circuit, the therapeutic current control network being configured to be operable in an operation mode of two modes of operation, the two modes comprising: a first mode in which the resonant electrical circuit is connected with the patient in a parallel configuration, and a second mode in which the resonant electrical circuit is connected with the patient in a series configuration; and a controller electrically coupled to the energy storage capacitor and the therapeutic current control network, wherein the controller is configured to, in connection with the energy provided by the energy storage capacitor that flows through the resonant electrical circuit, control operation of the at least one current control switch of the therapeutic current control network in delivering an electrotherapeutic waveform to the patient to correspond with a specified waveform.

In some examples, the operation mode is selected based at least in part on maximizing a value of a Q factor, wherein the value of the Q factor represents a measure of peak energy relative to dissipated energy with regard to the resonant electrical circuit, wherein the value of the Q factor is affected by the operation mode and a patient impedance. In some examples, wherein at least one of the first defibrillation shock and the second defibrillation shock, comprises a biphasic shock. In some examples, the first defibrillation shock comprises a positive phase shock vector and a negative phase shock vector, wherein a positive phase of the biphasic shock is delivered according to the positive phase shock vector, and wherein a negative phase of the biphasic shock is delivered according to the negative phase shock vector, and wherein the positive phase shock vector and the negative phase shock vector are in opposite directions along the first shock vector path. In some examples, at least one of: the first defibrillation shock and the second defibrillation shock, comprises a biphasic rectilinear current waveform. In some examples, at least one of the first defibrillation shock and the second defibrillation shock, comprises a truncated exponential current waveform.

In some examples, at least one of the first defibrillation shock and the second defibrillation shock, comprises a monophasic shock. In some examples, at least one of the first shock vector path and the second shock vector path defines an anterior-lateral path through a torso of the patient. In some examples, at least one of the first shock vector path and the second shock vector path defines an anterior-posterior path through a torso of the patient. In some examples, the first shock vector path defines an anterior-lateral path through a torso of the patient and the second shock vector path defines an anterior-posterior path through the torso of the patient. In some examples, the first shock vector path defines an anterior-posterior path through a torso of the patient and the second shock vector path defines an anterior-lateral path through the torso of the patient. In some examples, the first pair of electrodes and the second pair of electrodes have an electrode in common. In some examples, the at least three electrodes cable connected to the defibrillation accessory device via the at least one electrode port. In some examples, the system comprises a multielectrode pad configured to comprise a plurality of constituent electrodes, wherein each of the plurality constituent electrodes is for placement on a different area of the patient's torso, wherein at least one of the at least three electrodes is a constituent electrode of the plurality of constituent electrodes.

In some examples, the first defibrillator comprises a first controller, wherein the second defibrillator comprises a second controller, and wherein accessory device comprises a third controller. In some examples, the first defibrillator is configured to deliver the first defibrillation shock upon a corresponding selection by a care provider. In some examples, the selection comprises pressing of a button that causes delivery of the first defibrillation shock. In some examples, the system of claim H1, wherein, upon a corresponding selection by a care provider, the first defibrillator is configured to deliver the first defibrillation shock and the second defibrillator is configured to simultaneously deliver the second defibrillation shock. In some examples, the selection comprises pressing of a button that causes simultaneous delivery of the first defibrillation shock and the second defibrillation shock.

### BRIEF DESCRIPTION OF THE DRAWINGS

Various aspects of embodiments of the present disclosure are discussed below with reference to the accompanying figures, which are not intended to be drawn to scale. The figures are included for illustrative purposes and a further understanding of the various aspects and examples, and are incorporated in and constitute a part of this specification, but are not intended to limit the scope of the disclosure. The drawings, together with the remainder of the specification, serve to explain principles and operations of the described and claimed aspects and examples. In the figures, identical or nearly identical components that are illustrated in various figures may be represented by like numerals. For purposes of clarity, not every component may be labeled in every figure.

FIGS. 1A and 1B illustrate an example of double sequential external defibrillation (DSED).

FIG. 1C is a plot illustrating example first and second DSED biphasic shock waveforms, which may correspond with shocks 1 and 2 as illustrated in FIGS. 1A and 1B, in which delivery of shock 2 is initiated after completion of delivery of shock 1.

FIG. 1D is a plot illustrating example first and second DSED biphasic shock waveforms, which may correspond with shocks 1 and 2 as illustrated in FIGS. 1A and 1B, in which delivery of shock 2 is initiated during delivery of shock 1.

FIG. 1E is a plot illustrating example first and second double biphasic shock waveforms, which may correspond with shocks 1 and 2 as illustrated in FIGS. 1A and 1B, in which delivery of shock 2 and delivery of shock 1 are initiated simultaneously.

FIGS. 2A and 2B illustrate an example of DSED implemented using two defibrillators and a defibrillation accessor device.

FIG. 2C is a block diagram illustrating example components of a defibrillation accessory device.

FIG. 2D is a block diagram illustrating example components of a defibrillation accessory device as illustrated in FIGS. 2A-C.

FIG. 3 is a block diagram illustrating example high frequency impedance monitoring blocking subsystems.

FIGS. 4 and 5 are simplified circuit diagrams illustrating example programmable digital resistors of mirror transthoracic impedance subsystems.

FIG. 6A is a simplified circuit diagram illustrating an example protection circuit for a mirror transthoracic impedance subsystem.

FIGS. 6B and 6C are conceptual block diagrams illustrating example operation of a protection circuit.

FIGS. 7A and 7B are flow diagrams illustrating example operation of the protection circuit of FIG. 6A.

FIG. 8 is a simplified circuit diagram illustrating an example protection circuit for a mirror transthoracic impedance subsystem, using a 2 MOSFET implementation.

FIGS. 9A and 9B are flow diagrams illustrating example operation of the protection circuit of FIG. 8.

FIG. 10 is a flow diagram relating to an example of DSED.

FIGS. 11A and 11B illustrate an example DSED system including two defibrillators.

FIGS. 12A and 12B illustrate an example DSED system including one defibrillator and a defibrillation accessory device.

FIGS. 13A and 13B illustrate an example DSED system including one defibrillator.

FIGS. 14A and 14B illustrate an example of defibrillation with vector change, including use of a vector change accessory device.

FIG. 14C is a simplified circuit diagram illustrating an example vector change accessory device.

FIGS. 15A and 15B illustrate an example of defibrillation with vector change, including a defibrillator with a vector change system.

FIGS. 16A and 16B illustrate an example of DSED or defibrillation with vector change including a defibrillation accessory device, and including use of three electrodes.

FIGS. 17A and 17B illustrate an example of DSED or defibrillation with vector change including a defibrillator with a vector change system, and including use of three electrodes.

FIG. 18 provides a simplified illustration of an example of use of two multielectrode pads in DSED or vector change.

FIGS. 19A and 19B illustrate an example of DSED with a very small delay between first and second defibrillation shocks.

FIGS. 20A and 20B illustrate an example of DSED including two monophasic defibrillation shocks.

FIG. 21 is a block diagram illustrating example components of various devices described with reference to preceding figures.

### DETAILED DESCRIPTION

A delay, shock delay, or shock delay period may refer to, e.g., a delay between the initiation (also referred to as the start) of delivery of a first shock and the initiation of delivery of a second shock. A start-to-end delay may refer to, e.g., a delay between the completion of delivery of a first shock and the initiation of delivery of the second shock.

Some embodiments provide devices, systems, apparatuses, methods and computer readable media for, or relating, to double sequential external defibrillation (DSED) and for defibrillation with vector change, such as may include delivery of two (or more) shocks. Additionally, some embodiments include double (or more) external defibrillation where the two (or more) shocks (e.g., a first shock and a second shock) are delivered simultaneously. While many embodiments herein are described with reference to DSED, it is to be understood that, unless otherwise indicated or applicable, features described with reference to embodiments including DSED may also apply to embodiments including, for example, double external defibrillation with simultaneous delivery of (two or more) shocks.

For example, in double external defibrillation and defibrillation with vector change, two or more shocks may be delivered to a patient, where the second shock is delivered along a different vector line (e.g., a path determined or defined by a particular pair of electrodes) than the first shock. In DSED, delivery of the second shock may, e.g., occur following a shock delay period (e.g., on the order of milliseconds) following delivery of the first shock (e.g., initiation of delivery of the second shock may occur after a delay following initiation of delivery of the first shock). In some embodiments, delivery of the second shock may be initiated immediately following completion of delivery of the first shock (with no end-to-start delay). In some embodiments, initiation of the second shock may occur after an end-to-start delay following completion of the first shock. In some embodiments, at least a portion of a time period of delivery of the second shock may overlap with at least a portion of a time period of delivery of the first shock. In some embodiments, the second shock and the first shock may be initiated simultaneously (e.g., whether or not they are of the same duration and end at the same time or not). It is noted that, while two shocks may be referred to as a first and a second shock, in some embodiments, the first second and the second shock may be delivered, e.g., non-simultaneously or simultaneously. In defibrillation with vector change, the second shock may be, e.g., delivered seconds or minutes after the first shock is delivered, and may, e.g., result from a user selection, following delivery of the first shock, to deliver the second shock.

In some embodiments, DSED and defibrillation with vector change may be used for patients in refractory ventricular fibrillation (VF). In some examples, refractory VF may be defined or identified as occurring when a patient is not responding, or is not sufficiently responding, to defibrillation, or multiple defibrillation shocks, such as by remaining in VF after three defibrillation shocks. However, in other examples, refractory VF may be defined or identified differently. Some embodiments of DSED and defibrillation with vector change may be used for patients in refractory VF, or for refractory VF defined or identified in various different ways or by various different conditions or sets of conditions, or for patients not in refractory VF, or may be used under or based on particular criteria, conditions or circumstances in addition to, or other than, whether the patient is in refractory VF (e.g., criteria, conditions or circumstances relating to the system, the patient, treatment aspects, or the environment). It is noted that, in various embodiments, and in some circumstances where safe and appropriate, DSED or defibrillation with vector change may be used patients not in refractory VF, such as, for example, patients in ventricular tachycardia (VT).

In many cases, patients in refractory VF do not respond to further conventional shock protocols. For patients, or some patients, in refractory VF, DSED and/or defibrillation with vector change may be associated with better outcomes. It has been suggested that one reason for such improved outcomes may be that two or more shocks through the heart along different vector lines (e.g., different electrode pairs), and/or, in DSED, where the second shock is delivered after an extremely short shock delay period following the first shock (e.g., after 0-75 milliseconds), may defibrillate, or better defibrillate, a ventricle portion that may not be completely defibrillated by repeated shocks along the same vector line. In some embodiments, DSED may be associated with a greater probability of positive outcomes as compared with repeated non-DSED defibrillation shocks.

In various embodiments, the different vector lines may be established by different pairs of electrodes in different positions on the thorax of the patient, such as various anterior, lateral or posterior positions.

In some embodiments, a single defibrillator or two (or more) defibrillators may be used in providing the two (or more) shocks. For example, in some embodiments, a single defibrillator may be used in delivering each of the two shocks using a different pair of electrodes that define different shock vector paths. In some embodiments, two defibrillators may be used, where each defibrillator delivers one of the two shocks, and where each of the two shocks is delivered using a different pair of electrodes.

Furthermore, in some embodiments, a defibrillation accessory device (or more than one) may be used. Each of the defibrillator(s) may be cable connected to the defibrillation accessory device. Each of the two pairs of electrodes may also be connected to the defibrillation accessory device, where each connected defibrillator is used in delivering a shock using one of the connected pairs of electrodes. Electrical current for the defibrillation shocks may pass through the accessory device, and electronic circuitry thereof, before reaching the patient.

In some embodiments, in DSED, the defibrillator(s) and/or the defibrillation accessory device may provide for alternating patient transthoracic impedance monitoring, allowing for continuous patient transthoracic impedance monitoring associated with each of the two pairs of electrodes and associated vector paths. Measured patient transthoracic impedance values may be used in determining one or more electrical parameters associated with each of the defibrillation shocks. The alternating impedance monitoring may be used in providing solutions to problems that may be associated with simultaneous monitoring of patient transthoracic impedance associated with each of the two pairs of electrodes, which may lead to measurement inaccuracy.

In some embodiments, in DSED, various subsystems may be included, such as in a defibrillation accessory device, in one or several defibrillators, or distributed between multiple such devices. In some embodiments, these may include one or more of a second shock trigger subsystem, a high frequency impedance monitoring blocking subsystem (HFIMBS), a mirror transthoracic impedance subsystem (MTIS) and a protection circuit for an MTIS. The second shock trigger subsystem may, for example, be used in sensing delivery of the first shock and triggering delivery of the second shock to occur after a shock delay period.

The HFIMBS may be used, for example, in blocking continuous patient transthoracic impedance monitoring by each of two defibrillators, such as to prevent associated patient transthoracic impedance measurement error. While continuous patient transthoracic impedance monitoring by the defibrillators may be blocked, the MTIS may instead be used to provide impedance or resistance values to each of the defibrillators, relating to patient transthoracic impedance values associated with the respective pair of electrodes, which may be used to mirror the respective patient transthoracic impedance values. The MTIS may provide continuous alternating patient transthoracic impedance monitoring associated with each of the two pairs of electrodes, thus avoiding possible patient transthoracic impedance measurement error that may be caused by simultaneous patient transthoracic impedance monitoring associated with both pairs of electrodes, for example. The MTIS may use programmable resistors to provide mirrored patient transthoracic impedance or resistance values to each of the defibrillators.

Components of the MTIS, and connected components, may, however, require electrical protection from the high voltage applied during the defibrillation shocks. The protection circuit for the MTIS may be used in providing such protection, such as by providing series, rather than shunt, type electrical protection without being based on use of a protective capacitor, which may be unsuitable in embodiments described herein. For example, in some embodiments, the protection circuit may include use of four high voltage MOSFETS arranged in two cascode configurations of two MOSFETS each, or may include use of two suitably rated high voltage MOSFETS, if available.

In some embodiments, rather than, for example, four electrodes being used to provide the two electrode pairs, three electrodes may be used, with each of the two electrode pairs having an electrode in common.

In some embodiments, in defibrillation with vector change, a defibrillator or a defibrillation accessory device may include an electrode pair selection control, which may be used in switching an electrode pair used between the first defibrillation shock (along a first vector line) and the second defibrillation shock (along a second vector line), such as based on sensing of delivery of the first defibrillation shock.

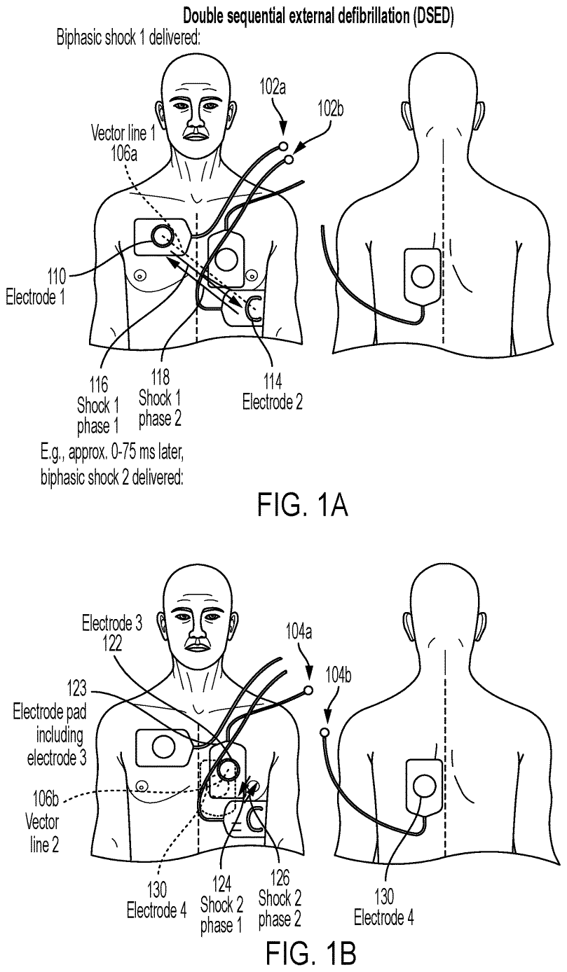

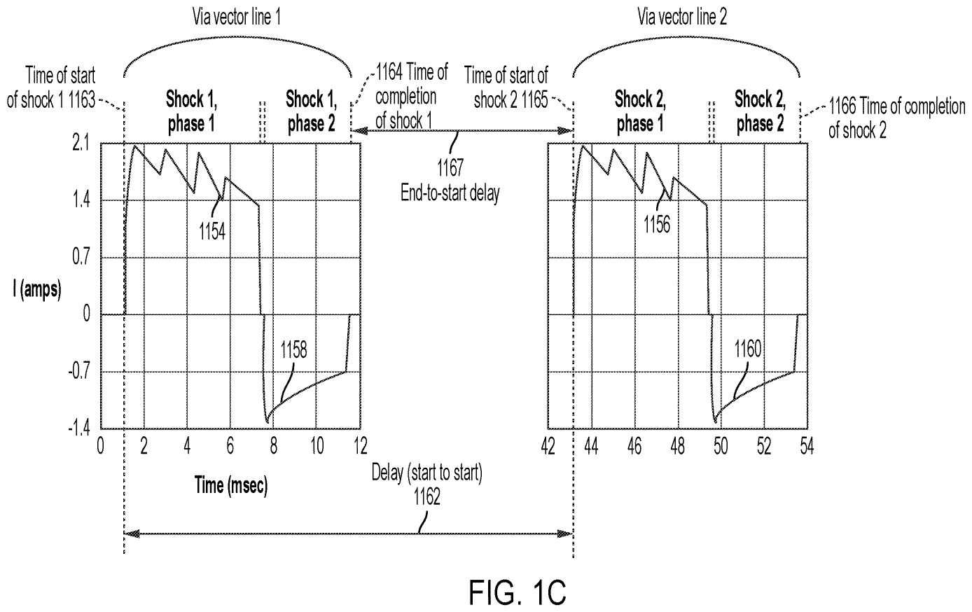

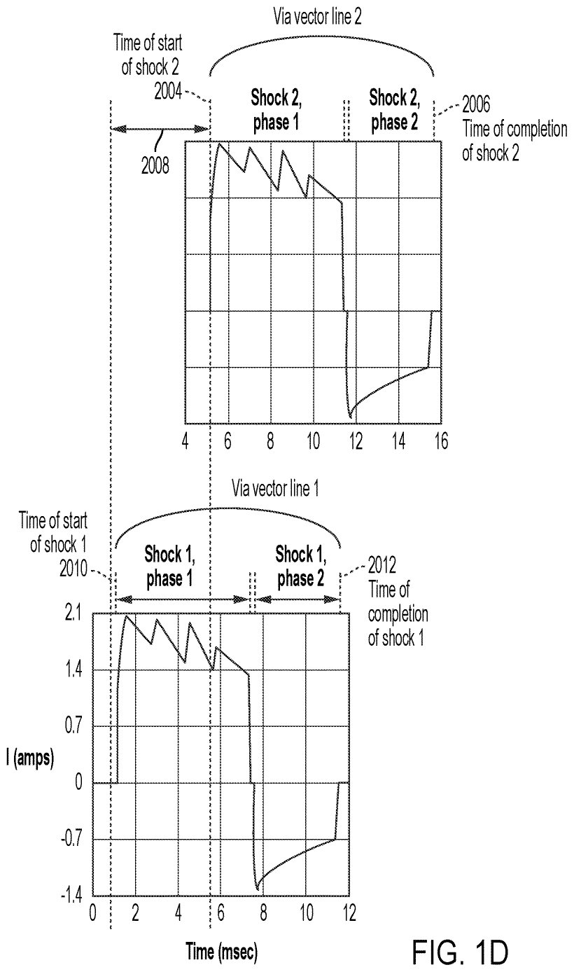

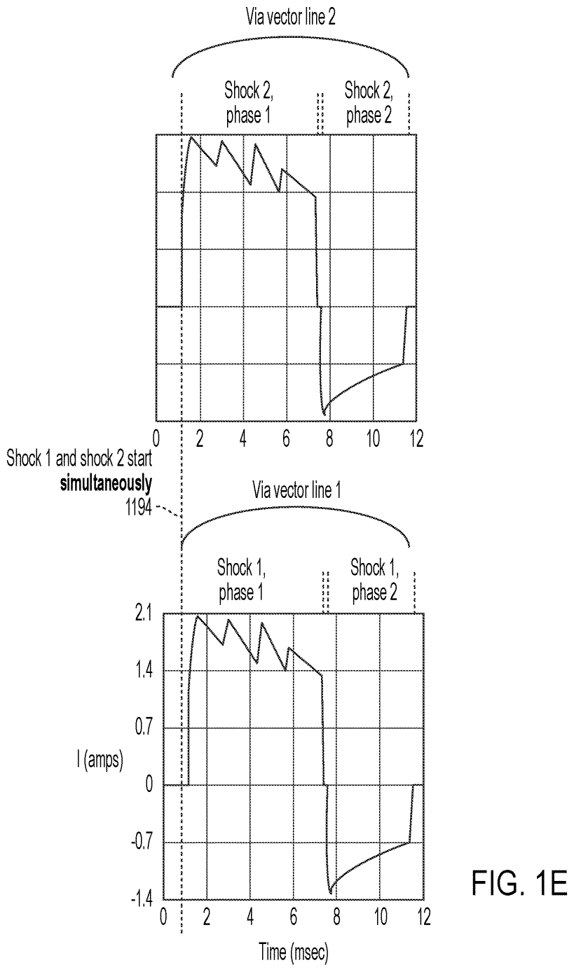

FIGS. 1A and 1B illustrate an example of double sequential external defibrillation (DSED). In the example depicted, two biphasic shocks are delivered, where delivery of the second shock (shock 2) occurs approximately 0-75 milliseconds after delivery of the first shock (shock 1), and where shock 1 and shock 2 are delivered along different vector lines.

In the example depicted, the first shock (shock 1) is delivered along vector line 1 106a and the second shock (shock 2) is delivered as a vector along vector line 2 106b. In this example, both shocks are biphasic, having a phase 1 (e.g., positive phase) and a phase 2 (e.g., negative phase). In phase 2, current flow is along the same vector line (as defined by the electrode pair) but in the opposite direction as phase 1. In FIG. 1A, the electrodes 110, 114, 122, 130, are included as part of electrode pads (e.g., electrode pad 123 includes electrode 3 122). It is noted that, in various embodiments, various different electrode pad types and electrode types and configurations may be used (e.g., one or more electrode pads may be used, each including one or more electrodes, or limited or no electrode pads may be used).

As depicted in FIG. 1A, shock 1 is delivered using electrode 1 110 and electrode 2 114, where electrode 1 110 is placed in an anterior position on the patient's torso (e.g., in this case, a right position on the patient's chest, in this case higher than/superior to the nipple), and electrode 2 114 is placed in a lateral position (e.g., in this case, toward the left side of the patient's chest, lower than/inferior to the nipple). As such, shock 1 is delivered along an anterior-lateral shock vector line. More specifically, shock 1 phase 1 116 is delivered in an anterior to lateral direction (along shock vector line 1 106a) and shock 1 phase 2 118 is delivered in a lateral to anterior direction (also along shock vector line 1 106a).

Shock 2 is delivered using electrode 3 122 and electrode 4 130, where electrode 3 122 is placed in an anterior position on the patient's torso (e.g., in this case, in a slightly left position on the patient's chest, generally slightly higher than/superior to the nipple), and electrode 4 130 is placed in a posterior position (e.g., in this case, slightly on the left side of the patient's back, generally close to/approximately level with the nipple, from a superior/inferior perspective). As such, shock 2 is delivered along an anterior-posterior shock vector line. More specifically, shock 2 phase 1 124 is delivered in an anterior to posterior direction (along shock vector line 2 106b) and shock 2 phase 2 126 is delivered in a posterior to anterior direction (also along shock vector line 2 106b).

It is noted that, in FIG. 1B, electrode 4 130, being positioned on the patient's back, may only be visible from a rear, or partially rear, view. As such, in the front view of FIG. 1B, electrode 3 130 is conceptually depicted in broken outline, although it may actually not be visible on the patient from a front view.

As depicted, pads including electrodes 1-4 110, 114, 122, 130 may be connected by electrically conductive cabling, in various embodiments, 102a, 102b, 104a. 104b, respectively, to, in various embodiments, one or more defibrillators or a defibrillation accessory device (which may itself, for example, by connected by cabling to one or more defibrillators), for example.

It is noted that the pad positions shown in figures herein are merely exemplary, and, in various embodiments, electrodes, which may be used to form electrode pairs, can be positioned in any of various positions on the patient, allowing any of many different vector lines and vectors (e.g., in either direction along a vector line), and any of many combinations and sequences of vector lines and vectors used for two (or more) defibrillation shocks, such as for DSED or defibrillation with vector change. As one example, in some embodiments, the positions of the electrodes 1-4 could instead be on the opposite sides of the patient as depicted in FIGS. 1A-B, e.g., with electrode 1 110 on a left, instead of right, position on the patient's chest, with electrode 2 114 being toward the right, instead of left, side of the patient's chest, with electrode 3 122 being in a slightly right, instead of left, position on the patient's chest, and with electrode 4 130 being slightly on the right, instead of left, side of the patient's chest. Of course, many variations and alternations of the positions of the electrodes 1-4 are possible, as well as variations of vector lines and vectors used, or sequences of vector lines and vectors used, or different positions, vector lines and vectors entirely.

In various examples, a first shock vector may pass from the left side to the right side of the patient, and the second shock vector may pass from the right side to the left side of the patient, or vice versa with respect to the sides, or in the opposite sequence. Furthermore, a first shock vector may pass from the front to the back of the patient, and a second shock vector from the back to the front of the patient, or vice versa with respect to front and back, or in the opposite sequence. In other examples, electrodes may be placed to be used to in providing a shock vector that passes through a left or right infraclavicular area or region, or to pass through a particular plane or region of the patient's thorax or of the patient's heart (e.g., the apex of the heart or another region or portion of the anatomy of the heart). Furthermore, electrodes could be places so as to provide a shock that passes through a particular quadrant, or other portion, of the patient's chest, heart or portion or anatomical feature of the heart.

In various embodiments, for the first and second shocks, electrode positions, and associated vector lines and vectors, may be selected based at least in part on particular anatomical features, regions or points along each vector. Furthermore, positioning may be based at least in part on other factors, such as the shock delay period or other factors relating to the shock, the shock waveform, the defibrillator(s), defibrillation accessor device or other devices, so as to optimize or maximize probably of successful outcomes.

FIG. 1C is a plot illustrating example first and second DSED biphasic shock waveforms, which may correspond with shocks 1 and 2 as illustrated in FIGS. 1A-B. More specifically, biphasic rectilinear waveforms are shown. However, in various embodiments. DSED or defibrillation with vector change may generate, include, use or employ any of various shock or pulse waveforms, such as truncated exponential waveforms, as well as monophasic waveforms. Additionally, in various embodiments, shock waveforms may be generated in various ways and using various electrical circuit topologies, such as may include use of a selected series of resistors or use of closed loop current control. Furthermore, in some embodiments, such topologies may include one or more resonant electrical circuits or resonant tanks. Examples of electrical circuit topologies and aspects of shock waveform generation, and other features, which may be included or used in embodiments herein, are described in U.S. patent application Ser. No. 17/331,275, filed on May 26, 2021 and entitled, “ELECTROTHERAPEUTIC WAVEFORM AND PULSE GENERATION AND DELIVERY SYSTEM AND METHOD” and International App. No. PCT/US2022/080792, entitled, “ELECTROTHERAPEUTIC WAVEFORM AND PULSE GENERATION,” each of which is hereby incorporated herein in their entirety.

As shown in FIG. 1C, phase 1 of shock 1 (e.g., the positive phase, during which current flows in an anterior to lateral direction) and phase 1 of shock 2 (e.g., the positive phase, during which current flows in an anterior to posterior direction) each include a series of ridges 1154, 1156, such as may, for example, be generated as a result of use of a series of resistors in the associated defibrillator electrical circuit topology (or, e.g., could be generated using closed loop control of defibrillation current). Phase 2 of shock 1 (e.g., the negative phase, during which current flows in a lateral to anterior direction) and phase 2 of shock 2 (e.g., the negative phase, during which current flows in a posterior to anterior direction) each include an upwardly sloping portion 1158, 1160.

Initiation of delivery of shock 2 may occur after a delay following initiation of delivery of shock 1 of, e.g., 0-75 milliseconds, 0-300 milliseconds, 125-175 milliseconds, 140-160 milliseconds, 150 milliseconds. 0.1-100 milliseconds, 0.01-50 milliseconds. 1-100 milliseconds, 1-50 milliseconds, 0.1-10 milliseconds, or 0-1 millisecond, 1-2 seconds, 2-5 seconds or 5-10 seconds. There may be an end-to-start delay (from the end of shock 1 to the start of shock 2) of, e.g., 0-75 milliseconds, 0-300 milliseconds, 125-175 milliseconds, 140-160 milliseconds, 150 milliseconds, 0.1-100 milliseconds, 0.01-50 milliseconds, 1-100 milliseconds. 1-50 milliseconds. 0.1-10 milliseconds, or 0-1 millisecond. 1-2 seconds, 2-5 seconds, or 5-10 seconds). In the example shown, there is a delay of approximately 42 milliseconds (from the start of shock 1 at approximately time 1 msec to the start of shock 2 at approximately time 43 msec) and an end-to-start delay (from the end of shock 1 to the start of shock 2) of approximately 31.5 seconds (from approximately time 11.5 msec to approximately time 43 msec).

FIG. 1D is a plot illustrating example first and second DSED biphasic shock waveforms, which may correspond with shock 1 (via vector line 1) and shock 2 (via vector line 2) as illustrated in FIGS. 1A and 1B, in which shock 2 is initiated during delivery of shock 1. As shown, shock 1 is initiated 2010 at approximately time 1 msec and ends 2012 at approximately time 11.5 msec (during delivery of shock 2). Shock 2 is initiated 2004 at approximately time 5.5 msec (during delivery of shock 1) and ends 2006 at approximately time 16 msec. As shown, shocks 1 and 2 are partially overlapping in periods of delivery (an overlap of approximately 6 milliseconds occurs from approximately time 5.5 msec to approximately time 11.5 msec). There is a delay 2008, from the initiation 2010 of shock 1 to the initiation 2004 of shock 2, of approximately 4.5 msec (from approximately time 1 msec to approximately time 5.5 msecs).

FIG. 1E is a plot illustrating example first and second DSED biphasic shock waveforms, which may correspond with shock 1 (via vector line 1) and shock 2 (via vector line 2) as illustrated in FIGS. 1A and 1B, in which shock 2 and shock 1 are initiated simultaneously 1194. In the embodiment shown, shocks 1 and 2 also end simultaneously; however, in some embodiments, shocks 1 and 2 may be of different durations (e.g., shock 1 may have a longer duration than shock 2, or shock 2 might have a longer duration than shock 1) and may not end simultaneously.

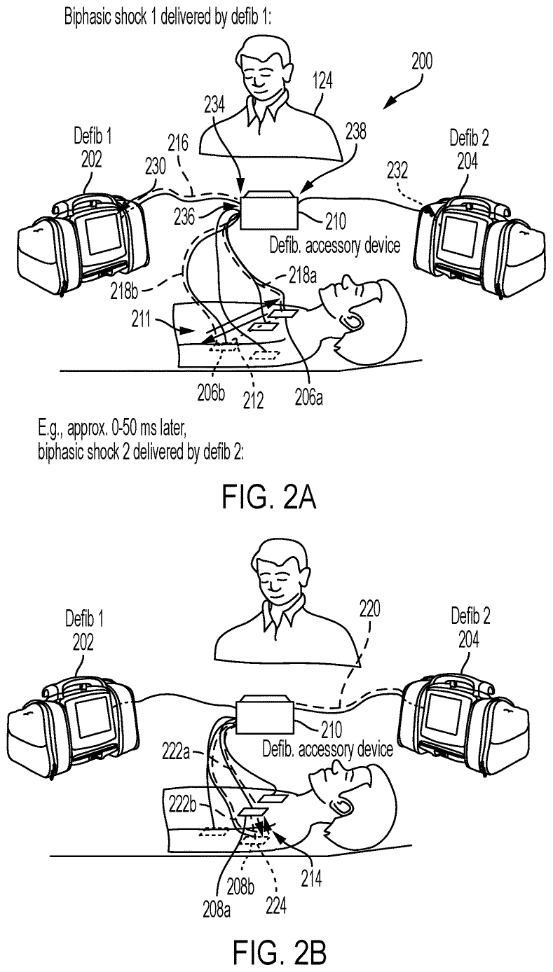

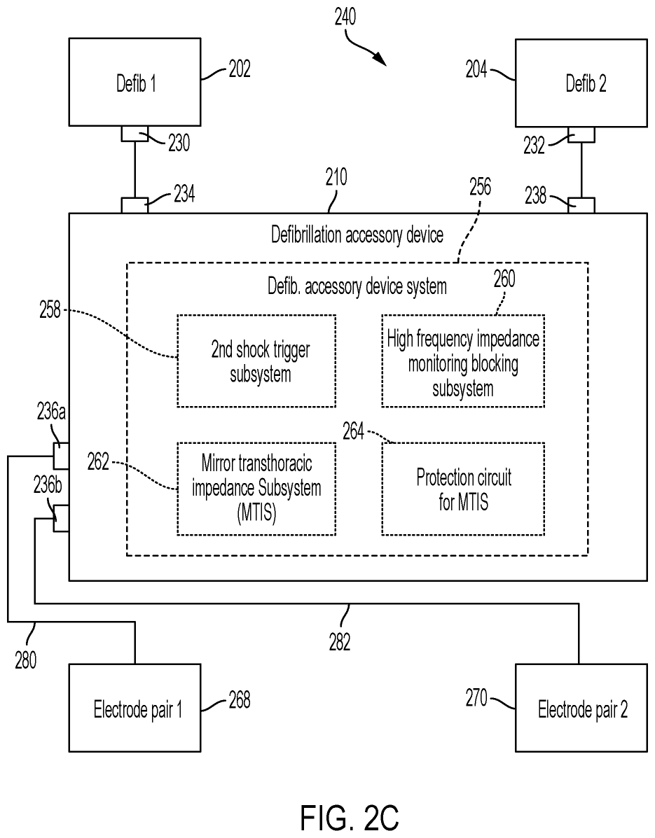

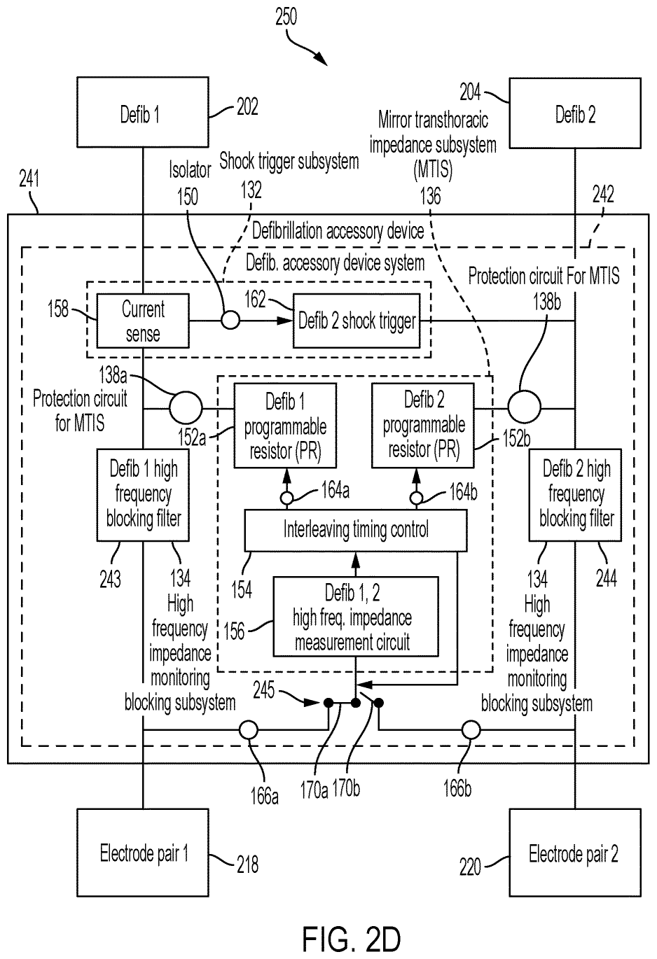

FIGS. 2A-B illustrate an example of DSED implemented using a system 200 including two defibrillators 202, 204 and a defibrillation accessory device 210. A user 124 (e.g., a care provider) is also shown (but two or more users may be present, for example). The system 200 (as well as other systems described herein) may, for example, be used in delivering shocks 1 and 2 as illustrated in previous FIGS. 1A-C. Each of the defibrillators 202, 204 may include, among other things, e.g., circuitry for generating defibrillation shock waveforms, a controller, a central processing units (CPU) and data storage.

As shown in FIG. 2A, defibrillator 1 202 delivers a first biphasic shock 211 along shock vector line 212 via electrode 206a (in an anterior position) and electrode 206b (in a lateral position). As conceptually illustrated by broken lines 216, 218a and 218b, shock 1 current flows, including via cabling, from defibrillator 1 202 through the defibrillation accessory device 210, including circuitry thereof, to the electrodes 206a, 206b. More specifically, during a positive phase of shock 1, current flows from defibrillator 1 202, through the defibrillation accessory device 210, to electrode 206a, through the torso of the patient, along shock vector line 212, to electrode 206b. During a negative phase of shock 1, current flows from defibrillator 1 202, through the defibrillation accessory device 210, to electrode 206b, through the torso of the patient, along shock vector line 212, to electrode 206a.

As shown in FIG. 2B, e.g., 0-75 milliseconds following the delivery of shock 1, defibrillator 2 204 delivers a second biphasic shock 214 along shock vector line 224 via electrode 208a (in an anterior position) and electrode 208b (in a posterior position). As conceptually illustrated by broken lines 220, 222a and 222b, shock 2 current flows, including via cabling, from defibrillator 2 204 through the defibrillation accessory device 210 to the electrodes 208a, 208b. More specifically, during a positive phase of shock 2, current flows from defibrillator 2 204, through the defibrillation accessory device 210, to electrode 208a, through the torso of the patient, along shock vector line 224, to electrode 208b. During a negative phase of shock 2, current flows from defibrillator 2 204, through the defibrillation accessory device 210, to electrode 208b, through the torso of the patient, along shock vector line 224, to electrode 208a.

In some embodiments, one or more defibrillators, such as defibrillators 1 202 and 2 204, may connect via electrically conductive cabling to a defibrillation accessory device. Furthermore, electrodes, such as electrodes 206a-b and 208a-b, may connect via electrically conductive cabling to a defibrillation accessory device, such as defibrillation accessory device 210. During a defibrillation shock, and potentially during, e.g., electrical monitoring of the patient (e.g., patient physiological monitoring such as electrocardiogram (ECG) monitoring), electrical current may flow through the defibrillation accessory device 210 before reaching the electrode(s) 206a-b. 208a-b and the patient, and, e.g., during monitoring, current may also flow from the electrode(s) 206a-b, 208a-b through the defibrillation accessory device 210 before reaching the defibrillators 1 202 and/or 2 204.

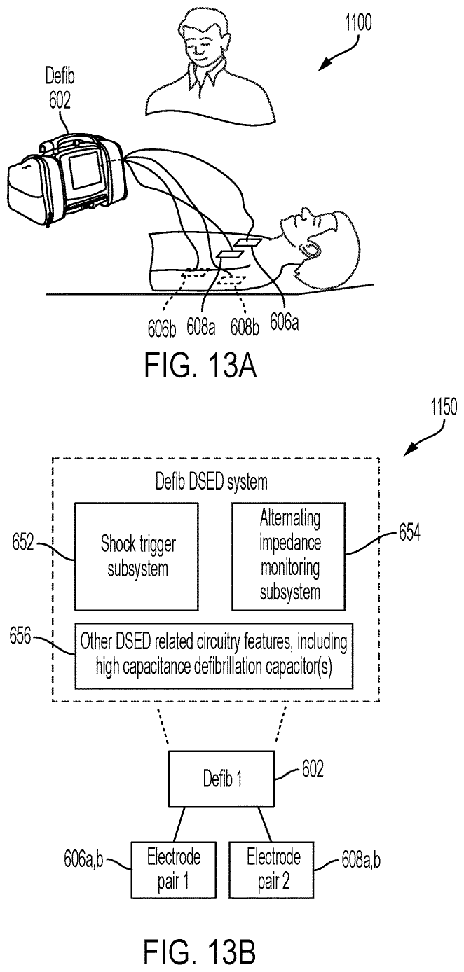

As shown in FIG. 2A and FIG. 2C, in some embodiments, for example, defibrillator 1 202 may include one or more ports 230 and the defibrillation accessory device 210 may include one or more ports 234, to allow cable connection to each other, where the ports can be of any of various types, and where the connection may be both physical and electrical. Similarly, defibrillator 2 204 may include one or more ports 232 and the defibrillation accessory device 210 may include one or more ports 238, to allow cable connection to each other (or, e.g., both defibrillators 202, 204 may connect via a single port, allowing multiple connections, on the defibrillation accessory device 210). Additionally, the defibrillation accessory device 210 may include one or more ports 236 for connection of cables to connect electrodes 206a-b and 208a-b, where the ports can be of any of various types, and where connection may be both physical and electrical. In the embodiment shown in FIG. 2C, two ports 236a-b are shown, with electrode pair 1 268 connecting by cable 280 to port 236a and electrode pair 2 270 connecting by cable 282 to port 236b. In various embodiments, however, two pairs of electrodes may connect with a defibrillation accessory device via. e.g., one port (or one port with multiple inputs), two ports (one port for each electrode pair), four ports (one port for each electrode cable), or in other ways. Furthermore, in embodiments in which two pairs of electrodes connect by cable to a single defibrillator (e.g., as shown in FIGS. 13A-B), the electrode pairs may connect with the defibrillator by one or more (e.g., one, two or four) ports on the defibrillator.

In some embodiments, defibrillators 1 202 and 2 204 may be automated external defibrillators (AED), or otherwise. In some embodiments, each of the defibrillators 1 202 and 2 204 may have implementation and operational features, or some such features, for allowing use of each of the defibrillators 202, 204 as independent defibrillators for use in non-DSED defibrillation treatment. In some embodiments, use of the accessory device 210 may allow use of such defibrillators (or such defibrillators with limited modifications or adaptations) in DSED.

For example, in some embodiments, a defibrillation accessory device may allow, or optimize use of, defibrillators for DSED, where the defibrillators may be older or otherwise not be implemented with features for DSED. In some embodiments, the subsystems of a defibrillation accessory device may be used in this regard. For example, such defibrillators may be set up to work individually and not in tandem with other defibrillators, and not already be preconfigured to have features to allow triggering of a second DSED shock after a shock delay period following a first DSED shock. Rather, they may be implemented such that a shock is delivered only upon pressing of a shock delivery button, for example. As such, a defibrillation accessory device may include a second shock delivery subsystem that may be configured to work with such defibrillators, such as by electrically triggering the second shock in a similar manner as would occur had a shock button been pressed, for example.

Additionally, such defibrillators may be implemented to perform continuous patient transthoracic impedance monitoring associated with the pair of electrodes that will be used to deliver a shock. However, if two such defibrillators are used in DSED, with both performing such monitoring associated with a different pair of electrodes, as mentioned above, this could result inaccurate patient transthoracic impedance measurements associated with each pair of electrodes, which could lead to incorrect defibrillation shock parameters, potentially leading to suboptimal defibrillation or injury to the patient. As such, in some embodiments, a defibrillation accessory device may include an MTIS to provide accurate mirror patient transthoracic impedance measurement values to each defibrillator, which may include each defibrillator electrically receiving such values in a manner that is similar to the manner in which they would receive patient transthoracic impedance or resistance values as measured by the defibrillators themselves. Additionally, the defibrillation accessory device may include other subsystems that may be required, such as a HFIMBS and a protection circuit for the MTIS. The protection circuit for the MTIS may be used, for example, in providing series protection for the programmable resistors of the MTIS (and potentially other connected components) while avoiding the problem of causing potential back-feeding of a shock, or portion thereof, to a defibrillator providing the shock, as described further herein.

As such, each of the subsystems of a defibrillation accessory device, as well as potentially other components thereof, such as other electrical protection components, may, in some embodiments, allow such defibrillators, which may be older or otherwise not intended to be implemented with features for DSED, to be safely used in DSED with no or minimal modifications. As described herein, however, in other embodiments, defibrillators themselves may include subsystems for allowing their use in DSED, with or without a defibrillation accessory device, whether such subsystems may be. e.g., included in the defibrillators as originally implemented or whether the defibrillators are modified later to include them.

For example, in some embodiments or implementations, a first care provider may arrive at an emergency scene and initially use defibrillator 1 204, after connecting electrodes 206a-b to defibrillator 1, to deliver defibrillation treatment. A second care provider may arrive shortly thereafter with defibrillator 2 204, along with electrodes 208a-b, which may or may not be attached to defibrillator 2 204. After, for example, three defibrillation shocks are delivered but fail to cause the patient to come out of VF (or, in some embodiments. VT), the patient may be considered to be in refractory VF, and it may be determined to then use DSED. At this point, prior to DSED treatment, defibrillators 1 202 and 2 204 may be connected by cabling, such as by one or more users, to the accessory device 210. Furthermore, electrodes 204a-b may be removed from defibrillator 1 202 (assuming, as in this example, that they were connected to defibrillator 1 202) and connected to the accessory device 210, and electrodes 206a-b may be removed from defibrillator 2 206 (again, assuming that they were connected to defibrillator 2 204) and connected to the accessory device 210, such as by one or more users. Prior to DSED treatment, the electrodes 204a-b and 206a-b are placed in the appropriate positions on the thorax of the patient.

As discussed herein, use of, e.g., the method as described above, including use of a defibrillation accessory device, may provide a safer and better alternative than, for example, simply using, or attempting to use, a second defibrillator to provide the second shock. For example, as described in detail herein, use of the defibrillation accessory device may avoid (or provide solutions to) problems including precisely timing the second defibrillation shock (such as by use of a second shock trigger subsystem), avoiding potential interference of monitoring or shocks applied by the defibrillators. These may include potentially inaccurate patient transthoracic impedance value measurements that may be caused by simultaneous monitoring by both defibrillators, and avoiding potential problems relating to compromised treatment, safety and damage to one or both of the defibrillators, such as older defibrillators or defibrillators that are otherwise not implemented with features for DSED, caused by the shocks inadvertently being applied to one or both of the defibrillators instead of completely or more completely to the patient.

The foregoing is one example of DSED. In other embodiments, as described herein, no accessory device may be used, and/or a single defibrillator (or more than two defibrillators) may be used. Furthermore, in various embodiments, one or more defibrillators used in DSED or defibrillation with vector change may have various degrees of adaptation or features for use in DSED or defibrillation with vector change. In some embodiments, an accessory device may allow one or more defibrillators that have relatively few, or no, DSED adaptations or features, to be used for DSED. In other embodiments, as described herein, one or more defibrillators may have some or all of the features needed for DSED, potentially without an accessory device, or with an accessory device with fewer or different features. In various embodiments, features to allow DSED or defibrillation with vector change may be included with an accessory device, or with one or both defibrillators, or distributed in various ways and combinations between an accessory device and one or both (or more than two) defibrillators, and/or other devices.

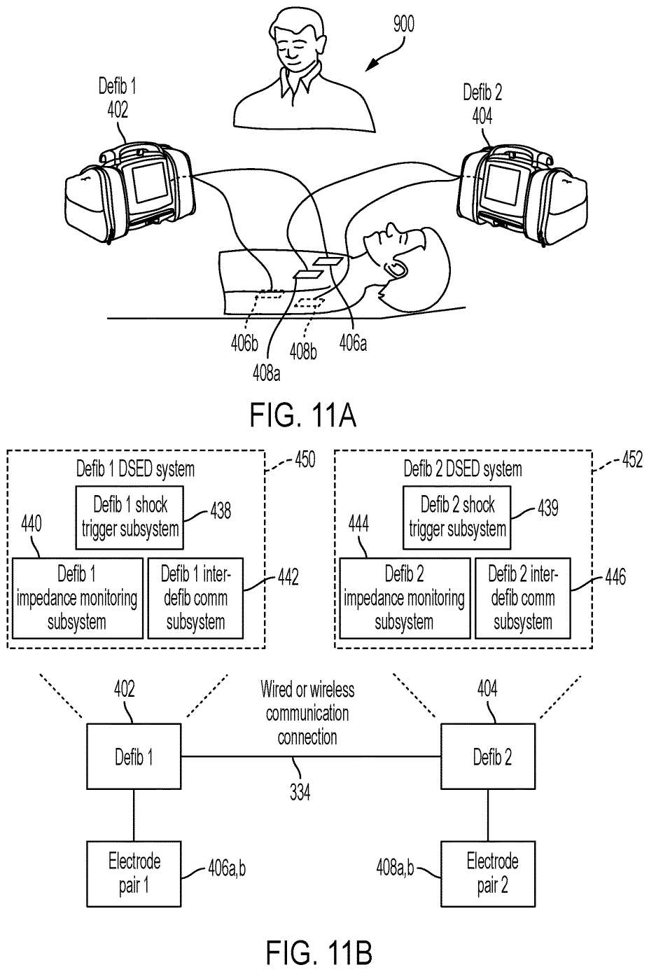

For example, as shown in FIGS. 11A-B (e.g., DSED with two defibrillators but no accessory device) and FIGS. 13A-B (e.g., DSED with one defibrillator but no accessory device), one or both defibrillators may include various features in this regard, such as may include subsystems, electrical circuitry and circuit components, software and algorithms. For example, one or both defibrillators may include a shock trigger subsystem that may be used in coordinating and timing of the DSED shocks. Furthermore, one or both defibrillators may include an alternating patient transthoracic impedance monitoring subsystem that may be used to allow for continuous, accurate, alternating patient transthoracic monitoring associated with each of two pairs of electrodes without interference. Still further, one or both of the defibrillators may include other features for use in DSED, such as potentially additional electrical protection measures and, in the case of a single defibrillator, ports for both pairs of electrodes and additional capacitor capacity for both shocks without recharging (e.g., a larger capacitor or several capacitors). Furthermore, with two defibrillators, each defibrillator may include hardware, circuitry and/or software to allow communication between the defibrillators in connection with DSED and coordination related to DSED (e.g., in alternating monitoring and shock delivery timing).

FIG. 2C is a block diagram illustrating example components of a DSED system 240 including a defibrillation accessory device, such as the defibrillation accessory device 210 as illustrated FIG. 2A-B. Connected with the defibrillation accessory device 210 are two defibrillators, such as defibrillators 1 202 and 2 204, as shown in FIGS. 2A-B, as well as electrode pair 1 (e.g. electrodes 206a-b as shown in FIGS. 2A-B) and electrode pair 2 (e.g. electrodes 208a-b as shown in FIGS. 2A-B). Furthermore, FIG. 2D is a block diagram illustrating a more detailed example of a DSED system 250 including components of an example 241 of the defibrillation accessory device 210 as shown in FIGS. 2A-C.

In the embodiment depicted in FIG. 2C, the defibrillation accessory device 210 includes a housing (which may include, or partially include, ports for connection of cables for connection of defibrillators 1 202 and 2 204 and electrode pairs 1 218 and 2 220) and a defibrillation accessory device system 256. The defibrillation accessory device 210 may also include, among other things, a controller, a central processing units (CPU) and data storage.

The defibrillation accessory device system 256 includes subsystems that may include a second shock trigger subsystem 258, a high frequency impedance monitoring blocking subsystem 260, a mirror transthoracic impedance subsystem (MTIS) 262 and a protection circuit 264 for the MTIS. In some embodiments, such as in which the defibrillation shocks are delivered simultaneously, the second shock trigger subsystem 238 may or may not be included. For example, in some embodiments in which the shocks are delivered simultaneously, the second shock trigger subsystem 258 may be used in triggering simultaneous delivery of the second shock with the first shock. However, in some embodiments in which the shocks are delivered simultaneously, the second shock trigger subsystem may not be included. For example, in some embodiments, a single trigger, such as a button press or other selection by a user, for example, may trigger delivery of both shocks simultaneously. While various embodiments depicted and described herein include a second shock trigger subsystem, in some embodiments, including variations of these, a second shock trigger subsystem may not be included.

The second shock trigger subsystem 258 may include circuitry for triggering the second shock of two DSED shocks, such as shock 2 as illustrated in previous figures. For example, in embodiments illustrated in previous figures, delivery of shock 2 may occur, e.g., 0-75 milliseconds after delivery of shock 1 (or, in various embodiments, e.g., 0-300 milliseconds, 125-175 milliseconds, 140-160 milliseconds, 150 milliseconds, 0.1-100 milliseconds. 0.01-50 milliseconds, 1-100 milliseconds. 1-50 milliseconds, 0.1-10 milliseconds, or 0-1 millisecond, 1-2 seconds. 2-5 seconds, or 5-10 seconds), which may be a shock delay period. For example, the second shock trigger subsystem 258 may detect delivery of shock 1 by defibrillator 1 and trigger delivery of shock 2 by defibrillator 2 to occur following the shock delay period. As such, a time of delivery of the second shock may be based at least in part on a time of delivery of the first shock and the shock delay period. However, in some embodiments, the second shock trigger subsystem 258 includes circuitry for triggering the second shock without a shock delay period, so that the second shock is delivered simultaneously with the first shock. For example, the second shock trigger subsystem may detect delivery of shock 1 and immediately trigger delivery of shock 2.

In various embodiments, the specified period time may be, e.g., preset, such as by being stored in a memory, or incorporated into a circuit topology or operation, of a device or component of the DSED system 240, such as a defibrillation accessory device, defibrillator, or other device, and/or by being preset by a user of one or more devices or components of the DSED system 240. For example, in some embodiments, one or more components of the DSED system 240, whether local or non-local, may include one or more controls to allow user specification or selection of the shock delay period prior to DSED using the shock delay period. For example, such controls may include physical controls (e.g., one or more buttons or scroll wheels) or graphical user interface (GUI) based controls (e.g., one or more user-interactive display screens, such as LED based display screens). In various embodiments, such controls may be included in any of various devices or components of the DSED system 240, which may include defibrillators 1 202 and 2 204, the defibrillation accessory device 210, and/or one or more other devices, such as a computerized device (e.g., smartphone, portable, handheld or tablet computing device), other medical device, or patient monitor, for example. Furthermore, in some embodiments, the DSED system 240 may include one or more non-local devices or systems, such as one or more remote computing systems or platforms.

In the example depicted in FIG. 2C, the defibrillation accessory device system 256 also includes high frequency impedance monitoring blocking subsystem 260, MTIS 262 and the protection circuit 264 for the MTIS. In defibrillation, a defibrillator may be preconfigured so as to continuously send electrical current through the thorax of the patient via the electrode pair that may be used in sending a defibrillation shock, for measurement of patient transthoracic impedance. The measured transthoracic impedance value may be used in determination of at least one parameter, e.g., an electrical or circuit related parameter, for the defibrillation shock. Patient impedance can vary between different paths through the thorax of the patient, and therefore between different electrode pairs. Still further, even for a particular path and pair of electrodes, patient impedance can vary over time, and even over short periods of time (e.g., 1 second or less). As such, in defibrillation, a defibrillator may continuously send high frequency patient impedance monitoring current via an electrode pair to be used for a potential defibrillation shock.

In some embodiments, where two defibrillators are used in DSED, each defibrillator may continuously send such monitoring current via the corresponding pair of electrodes. For example, in FIGS. 2A-B, defibrillator 1 202 may send such monitoring current, or excitation current, via electrodes 206a-b, and defibrillator 2 204 may send such monitoring current via electrodes 208a-b. In FIG. 2C, defibrillator 1 202 may send such monitoring current via electrode pair 1 268 and defibrillator 2 270 may send such monitoring current via electrode pair 2 270. This, however, can present a technical problem, in that simultaneous patient transthoracic impedance monitoring and associated measurement by both defibrillators may cause interference with the accuracy of each such measurement. This, in turn, can lead to inaccurate patient impedance measurements, which, in turn, can lead to improper shock or shock waveform parameters, such as an improper amount of energy being delivered to the patient. This can lead to suboptimal patient treatment, poor outcomes or serious injury to the patient.

For example, the two defibrillators may monitor using electrical frequencies that are not locked relative to each other, so that, at a given time, the relationship between the frequencies may be unpredictable or undefined, which can, in turn, lead to an unpredictable interference effect, and cause inaccuracy, for each patient transthoracic impedance measurement. For example, if the two sources of monitoring current happen to be in phase with each other, that can cause an increase in current density in the thorax of the patient, which can cause an increase in a detected voltage drop, which can, in turn, lead to inaccurately low determinations or estimates of patient transthoracic impedance. Conversely, if the two sources of monitoring current happen to be in an exactly opposite phase relationship, this may result in overall shorter current paths through the thorax of the patient, which can create a decrease in a detected voltage drop, which can, in turn, lead to an inaccurately high determinations or estimates of patient transthoracic impedance. This can lead to. e.g., an improper amount of energy being delivered to the patient from the shock, which can reduce the probably of success of potentially life-saving treatment, and may be physically dangerous or life-threatening to the patient. Furthermore, in some implementations, such inaccuracies may improperly trigger alarms, such as by leading to inaccurately small patient transthoracic impedance values that may improperly trigger a short circuit alarm, which can delay or prevent potentially life-saving defibrillation treatment.

Additionally, DSED with two defibrillators, for example, can cause the problem of risk of damage to one or both of the defibrillators as a result to the defibrillation shocks being delivered so closely in time to each other. For example, a defibrillator may include a mechanical patient isolation relay that may close very shortly prior to shock delivery, and re-open very shortly after, which may be achieved using solid state switching devices. The open relay state may prevent current from any potential external source from, e.g., reaching or back-feeding into these solid state devices. For example, in DSED, as illustrated in previous figures, if shock 2 was delivered from defibrillator 2 prior to the opening of the patient relay of defibrillator 1, this may lead to a portion of the energy of shock 2 back-feeding to the circuitry of defibrillator 1, potentially damaging, or permanently damaging, defibrillator 1, thus potentially rendering defibrillator 1 incapable of delivering life-saving treatment.

Some embodiments provide solutions to such technical problems as described above, such as, for example, by utilization of defibrillation accessory devices, and/or one or several defibrillators, including appropriate systems or subsystems (e.g., as illustrated in FIG. 2C, for a defibrillation accessory device, and as illustrated in later figures for defibrillation accessory devices and defibrillators). These may, for example, be used in preventing inaccurate patient transthoracic impedance measurements and associated improper defibrillation shocks or shock characteristics (e.g., delivered energy and waveforms) and/or inappropriate alarms, as well as in providing protection against shock back-feeding and associated potential defibrillator damage. For example some embodiments include blocking of patient transthoracic impedance monitoring by each of two defibrillators used in DSED (e.g., which may include use of a high frequency impedance monitoring blocking subsystem 260, as illustrated in FIG. 2C), providing of mirror transthoracic patient transthoracic impedance monitoring (e.g., which may include use of a mirror transthoracic impedance subsystem (MTIS) 262, as illustrated in FIG. 2C) and/or electrical protection or isolation subsystems or features (e.g., which may include use of a protection circuit 264 for the MTIS, and or other electrical isolation or protection features or components, such as those illustrated and described herein with reference to FIG. 2D), as well as potentially other features, such as particular patient relays or patient relay features, and/or second shock trigger features (e.g., a second shock trigger subsystem 258, as illustrated in FIG. 2C). Furthermore, some embodiments include related or other features, as illustrated and described with reference to other figures, including for systems with or without a defibrillation accessory device, and systems with one or several defibrillators, for example.

It is noted, however, that, in various embodiments, various approaches may be taken, such as to provide a solution to the problem associated with simultaneous high frequency patient transthoracic impedance monitoring. For example, in some embodiments, an accessory device, or two defibrillators without an accessory device, may be implemented to send simultaneous or overlapping patient impedance monitoring signaling, but use signaling frequencies that are sufficiently different so as to minimize interference and introduction of measurement inaccuracy or potential measurement inaccuracy.

As illustrated in FIG. 2C, the second shock trigger subsystem 258 may be used, in DSED, in triggering delivery of the second defibrillation shock (e.g., shock 2, delivered by defibrillator 2, as illustrated in previous figures), such as following a shock delay period after delivery of shock 1. As previously described, in various embodiments, the specified period time may be, e.g., preset, such as by being stored in a memory or incorporated into a circuit topology or operation of a defibrillation accessory device or other device or controller thereof, and/or preset by a user of one or more components of a DSED system.

As illustrated in FIG. 2D, in some embodiments, upon detection, by the current sense 158, of the shock trigger subsystem 132, of, e.g., delivery of shock 1 by defibrillator 1 202, the shock trigger 162, of the shock trigger subsystem 132, triggers, e.g., delivery of shock 2 by defibrillator 2 204 to occur after the shock delay period. The current sense 158, which is placed in an electrical path between defibrillator 1 202 and electrode pair 1 218 (and the patient), may include an electrical circuit capable of sensing current of (or, in other embodiments, voltage or another electrical parameter that is characteristic of) the shock 1, and thereby detect delivery of shock 1. For example, in some embodiments, the current sense 158 may include a current sense transformer.

As illustrated in FIG. 2D, positioned between the current sense 158 and the shock trigger 162 may be an electrical isolator circuit 150, such as an isolation transformer circuit (e.g., a circuit including a transformer circuit), which may be able to withstand, and operate correctly when exposed to, high current and high voltage, such as may be associated with a defibrillation shock. For example, in some embodiments, the isolation transformer circuit includes a current transformer circuit that blocks or mostly blocks the defibrillation current, providing potential protection for defibrillator 2 204, but generates a signal that is sent to the shock trigger 162. However, in some embodiments, a current sense transformer, as mentioned above, may serve as an electrical isolator circuit, in which case, the electrical isolator circuit 150 may be omitted, or both the current sense transformer and the electrical isolator circuit 150 may contribute to electrical isolation. Upon receiving the signal, the shock trigger 162 may generate and send a signal to defibrillator 2 204 to cause defibrillator 2 204 to deliver shock 2 following the shock delay period. In some embodiments, the shock delay period is long enough such that, before delivery of shock 2 by defibrillator 2 204, delivery of shock 1 is completed and the patient relay of defibrillator 1 202 has opened.

In some embodiments, defibrillator 1 202 may be considered the master defibrillator and defibrillator 2 204 may be considered the slave defibrillator. In some embodiments, the master-slave relationship between the defibrillators 202, 204 may include that, in DSED, delivery of shock 1 by defibrillator 1 202, and the timing thereof, is independent of delivery of shock 2 by defibrillator 204, but delivery of shock 2 by defibrillator 2 204 is dependent at least in part on delivery of shock 1 by defibrillator 1 202 and the timing thereof (such as by being delivered following the shock delay period after delivery of shock 1). For example, in some embodiments, in DSED, the delivery of shock 1 by defibrillator 1 202 may be triggered by a user selection, such as by selecting or pressing a button or using another control on defibrillator 1 to cause delivery of shock 1, when appropriate or allowed by defibrillator 1 or the DSED system 250. The shock trigger subsystem 132 may then detect delivery of shock 1 and trigger delivery of shock 2 by defibrillator 2 to occur following the shock delay period.

In some embodiments, a DSED system, e.g., system 250, may include, e.g., a DSED mode, which may include controls or user interface based features relating to DSED, which may, e.g., be implemented using software and algorithms. For example, in various embodiments, a DSED mode may entered into by a user, such as via a control, user interface or configuration screen, such as may be included on one or both of the defibrillators (if two are included), the defibrillation accessory device (if included), or another device (e.g., a tablet), or may be entered into when particular conditions relating to DSED are present, or both. In some embodiments, entry into DSED mode may be offered as a selection to be entered into when one or more conditions relating to or necessary for DSED are present, such as when the defibrillators are connected to the defibrillation accessory device, and/or when electrodes are connected to the defibrillation accessory device, or may be entered upon satisfaction of such condition(s) without user selection, and appropriate notifications may be provided on displays of the defibrillators, for example. In DSED embodiments with two defibrillators and no accessory device, in some embodiments, connection of the defibrillators with each other may trigger DSED mode.

In some embodiments, for example, notification displays may be presented on display screens of each of the defibrillators confirming entry into DSED mode, whether automatically or via user selection. Once in DSED mode, DSED features and options may be provided to the user. For example, after DSED mode in entered, the slave defibrillator may be ready to deliver the second shock after the proper amount of time has passed following the time when the DSED shock button is pressed on the master defibrillator, such that the second shock is delivered after the shock delay period following delivery of the first shock.

In other embodiments, the defibrillation accessory device may include a shock button which, when pressed, or when available and pressed, causes shock delivery from both defibrillators, by causing delivery of the first shock and delivery of the second shock after the shock delay period. Furthermore, in some embodiments, the defibrillation accessory device may include controls and/or a user interface, and may provide user messages, options and notifications relating to DSED.

The shock trigger 162 may include one or more circuits and, in some embodiments, may include a controller, and/or may operate in connection or communication with a controller of another device of the DSED system 250. In various embodiments, the shock trigger 162, as well as other devices in the DSED system 250, may operate in various different ways. For example, in some embodiments, upon receiving signaling of occurrence of delivery of shock 1, the shock trigger 162 may (and may be implemented or programmed so as to) allow the shock delay period to pass and then send a signal to defibrillator 2 204 to deliver shock 2. In other embodiments, the shock trigger 162 may immediately send a signal to defibrillator 2 to cause defibrillator 2 204 to deliver shock 2 following the shock delay period, where defibrillator 2 204 may be implemented or programmed to allow the shock delay period to pass and then deliver shock 2.

As described herein, in various embodiments, one or more defibrillators used in DSED, such as defibrillators 1 202 and 2 204, may or may not have various features and adaptations for use in DSED, or an accessory device may be used to supply some or all such features or allow defibrillators to be used in DSED even if they have limited or no such features. For example, in some embodiments, defibrillator 2 204 may be implemented specifically to receive a signal from the accessory device 210 to trigger DSED shock 2, separate from any triggering system that may allow triggering of a non-DSED shock, such as a triggering system in which a user presses a shock button to cause delivery of a non-DSED shock, and the accessory device 210 may be implemented to signal accordingly. However, in some embodiments, the shock trigger 162 may operate by signaling defibrillator 2 204 as if, or partly as if, the signaling was a result of a shock button being pressed for a non-DSED shock, such that the signal is internally received by defibrillator 2 204 in a way that is identical or similar to that of receipt of a signal resulting from the pressing of a shock button for delivery of a non-DSED shock, and the accessory device 210 may be implemented to signal accordingly. As such, in various embodiments, the defibrillation accessory device 241 may signal defibrillator 2 204 in various ways, and the signal may be received by defibrillator 2 204 in various ways. Furthermore, in some embodiments, various features and/or subsystems of an accessory device (e.g., a second shock trigger subsystem, a high frequency impedance monitoring blocking subsystem, and a mirror transthoracic impedance subsystem) may allow one or more defibrillators to be used in DSED even if they have limited or no features or adaptations for use in DSED, or otherwise.

In some embodiments, delivery of a single biphasic defibrillation shock, such as shock 1 or shock 2, may occur over a period of several milliseconds, such as, e.g., 2-50, 2-25, 5-15, 8-12 or approximately 10 milliseconds. In some embodiments, shock 2 is delivered such that a time of the start of delivery of shock 2 (that is, a time of the start of the several millisecond time period of delivery of shock 2) occurs following the shock delay period, e.g., 0-75 milliseconds, from a time of the completion of delivery of shock 1 (that is, a time of the completion of the several millisecond (or, e.g., up to 50 milliseconds) time period of delivery of shock 1). Referring to FIG. 1C, broken line 1163 marks the time of the start of shock 1 (when shock 1 current starts) and broken line 1164 marks the time of completion of shock 1 (when shock 1 current ends). Similarly, broken line 1165 marks the time of the start of shock 2 (when shock 2 current starts) and broken line 1166 marks the time of completion of shock 2 (when shock 2 current ends). As shown in FIG. 1C, the time 1165 of the start of shock 2 may occur following the shock delay period of, e.g., 0-75 milliseconds, after the time 1164 of completion of shock 1.

In some embodiments, the shock trigger 162 may be implemented or programmed such that the shock trigger 162 sends an electrical signal to defibrillator 2 204 to deliver shock 2 such that shock 2 is delivered after the shock delay period. As shown in FIG. 2D, the high frequency impedance monitoring blocking subsystem 134 includes a defibrillator 1 202 high frequency blocking filter 243 (blocking filter 243) and a defibrillator 2 204 high frequency blocking filter 244 (blocking filter 244). Blocking filter 243 is electrically coupled between defibrillator 1 202 and electrode pair 1 218 (with the current sense 158 coupled between the blocking filter 244 and defibrillator 1 202), and blocking filter 244 is electrically coupled between defibrillator 2 204 and electrode pair 2 220. In the embodiment depicted, defibrillators 1 202 and 2 204 are each implemented to continuously send high frequency impedance monitoring electrical signaling. However, as described in detail herein, simultaneous, or overlapping in time, monitoring by both defibrillators 202, 204 can cause inaccurate patient impedance measurements, which can lead to improper defibrillation shock parameter(s), such as an improper amount of delivered energy to the patient. Therefore, the blocking filters 243, 244 are used to block such high frequency electrical monitoring by the defibrillators 202, 204. In the embodiment shown in FIG. 2D, and as described in detail with regard to later figures, the MTIS 136 is instead used for patient transthoracic impedance monitoring to be supplied to each of the defibrillators 202, 204, such as by supplying an impedance or resistance value to mirror the patient transthoracic impedance.

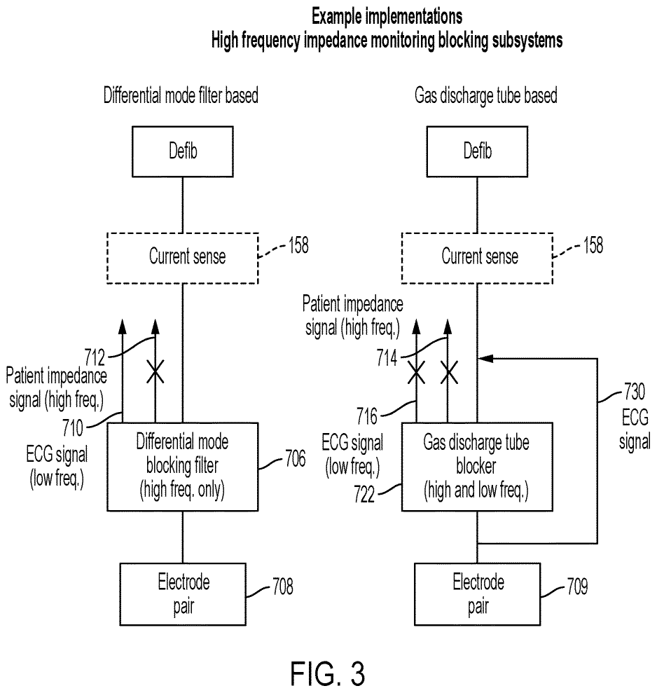

FIG. 3 is a block diagram illustrating example high frequency impedance monitoring blocking subsystems (blocking subsystems), including a differential mode filter based embodiment 706 and a gas discharge tube based embodiment 722. In various embodiments, in the example shown in FIG. 2D, either type of blocking subsystem 706, 722 (or another type) may be used with, e.g., defibrillator 1 202 and/or defibrillator 2 204 and either may be used as defibrillator high frequency blocking filters 243, 244 as shown in FIG. 2D, for example. Also as shown in FIG. 2D, in the case of defibrillator 1 202, a current sense 158 is coupled between the blocking subsystem 706, 722 and defibrillator 1 202, whereas, in the case of defibrillator 2 204, no current sense 158 is coupled between the blocking subsystem 706, 722 and defibrillator 2 204.

In the differential mode filter based blocking subsystem 706, a differential mode filter is used to block (or sufficiently block or suppress) the high frequency monitoring (excitation) current sent by the defibrillator for patient transthoracic impedance monitoring (which, as described herein, may be replaced by the operation of an MTIS). In some embodiments, the differential mode filter may be implemented including re-configuring a common mode filter to reverse a selected one or more of its windings, converting it into a differential mode filter, and causing it to differentially and selectively block high frequency signaling, for example. As shown in FIG. 3, this blocking filter 706 blocks 712 high frequency signaling from the defibrillator, but allows 710 (or mostly or sufficiently allows) low (or less high) frequency signaling to pass. In some embodiments, this provides a solution to the technical problem of allowing low frequency patient ECG signaling to pass from the electrode pair 708 to the defibrillator (and/or to allow other low or lower frequency monitoring or signaling to pass in either direction), while still blocking defibrillator high frequency patient impedance monitoring.

In another embodiment, in the gas discharge tube blocking subsystem 722, one or more gas discharge tubes are used to block (or sufficiently block or suppress) the defibrillator high frequency patient impedance monitoring. However, this blocking subsystem 722 may block 714, 716 both high and low frequency signaling to and from the defibrillator, which can lead to the problem that low or lower frequency signaling and monitoring, such as ECG monitoring, as mentioned, is also blocked. However, some embodiments provide a solution to the technical problem that the use of the gas discharge tube based blocking subsystem 722 presents in this regard. Specifically, in some embodiments, an electrical circuit 730 providing a circumvent may be used. The electrical circuit 730 may provide a low or lower frequency signal path that circumvents the gas discharge tube based blocking subsystem 730, thereby allowing, or sufficiently allowing, low frequency signaling, such as ECG signaling to pass, while still allowing blocking, or sufficient blocking, of high frequency patient transthoracic impedance monitoring by the defibrillator.

As shown in FIG. 2D, the mirror transthoracic impedance subsystem (MTIS) 136 may be used to provide patient transthoracic impedance or resistance values to defibrillators 1 202 and 2 204, to be used, e.g., in determining one or more parameters of defibrillation shocks provided as part of DSED, for example. Since defibrillator patient transthoracic impedance monitoring may be blocked (e.g., to avoid inaccurate patient transthoracic impedance measurements by the defibrillators 202, 204), the MTIS 136 may be used. e.g., to monitor and provide or allow for accurate such measurements, while providing a solution to overcome the technical problem of possible inaccuracy introduced as a result of potential simultaneous patient transthoracic impedance monitoring associated with each of the electrode pairs 218, 220 (if not blocked). More particularly, in the embodiment as depicted in FIG. 2D, the programmable digital resistors 152a, b (e.g., programmable digital resistor circuits) are used in providing high frequency resistance values to provide, or allow determination of, high frequency impedance or resistance values to defibrillators 1 202 and 2 204, respectively, associated with patient transthoracic impedance associated with the electrode pairs 1 218 and 2 220, respectively. The associated impedance values may be used by each of the defibrillators 202, 204, respectively, as representative of or corresponding with patient transthoracic impedance or resistance values, such as in determining one or more electrical parameters of a DSED shock delivered to the patient via electrode pairs 1 218 and 2 220, respectively.

As depicted in FIG. 2D, an interleaving timing control 154 (e.g., an interleaving timing control circuit) may be used in alternating or toggling between monitoring for and measurement of patient transthoracic impedance associated with electrode pairs 1 218 and 2 220, where the measurement itself occurs with the use of the high frequency impedance measurement circuit 156. This alternating monitoring and measurement may be used in providing a solution to the technical problem of providing ongoing (potentially changing over time) patient transthoracic impedance measurements to each of the defibrillators 202, 204, while yet avoiding simultaneous monitoring and its associated potential interference with measurement accuracy and/or other risks. For example, in some embodiments, the interleaving timing control 154 may alternate or toggle between measurements associated with electrode pairs 1 218 and 2 220 every, e.g., 0.5 seconds (or, e.g., 0.01-10 seconds, 0.1-3 seconds, 0.1-1 second, or 0.3-0.7 second) Particularly, in the embodiment depicted, the interleaving timing control 154 may control the toggling by controlling operation of a switch or switch network 245 of one or more switches to select the appropriate electrical pathway corresponding with the appropriate one of the electrode pairs 218, 220. For example, as depicted, switch 170a is closed and switch 170b is open, selecting electrode pair 1. Even though patient transthoracic can vary between electrode pairs (electrical paths) and over time, this provides one example of a solution in facilitating continuously (e.g., ongoingly and very frequently updated) providing accurate patient transthoracic impedance or resistance values to both defibrillators 202, 204, for use, e.g., in DSED, using different electrode pairs and different shock vectors. In various embodiments, various timing protocols may be used, including, in some embodiments, protocols where the toggling time may vary depending on particular parameters or circumstances, or where the time spent monitoring the impedance associated with each of the two pairs of electrodes is not equal. For example, in some embodiments, if CPR chest compressions are being performed, or detected as being performed, than toggling time may be decreased substantially, e.g., from 5 seconds if no compressions are detected to 0.1 second if chest compressions are detected.

As depicted in FIG. 2D, the interleaving timing control 154 controls the switch network 245 in order to determine whether patient transthoracic impedance signaling flows from electrode pair 1 218 or 2 220. Furthermore, the interleaving timing control circuit 154 signals the appropriate one of the programmable resistors 152a, b. Specifically, when the path from electrode pair 1 218 is adjusted, the path to programmable resistor 152a is also adjusted, which leads to defibrillator 1 202 to provide patient transthoracic impedance or resistance values to defibrillator 1 202 for use in possible DSED shock 1. Alternately, when the path from electrode pair 2 220 is adjusted, the path to programmable resistor 152b is also adjusted, which leads to defibrillator 2 204 to provide patient transthoracic impedance or resistance values to defibrillator 2 204 for use in possible DSED shock 2. In some embodiments, defibrillator 1 202 continuously monitors programmable resistor 152a, and defibrillator 2 204 continuously monitors programmable resistor 2 152b, and the programmable resistors 152a,b are periodically updated. As shown, electrical isolators, such as capacitive isolators 166a,b (or capacitive isolator circuits) are included, in an electrical leading to the measurement circuit 156 of the MTIS 136 from defibrillators 1 202 and 2 204 and electrode pairs 1 218 and 2 220, which may be used in protecting the measurement circuit 156 and the MTIS 136 from potential damage from delivered defibrillation shocks. Additionally, as shown, electrical isolators, such as digital isolators 164a, b may be coupled within the MTIS 136 between the interleaving timing control 154 and the programmable resistors 152a,b, respectively. Furthermore, electrical protection circuits 138a,b (as described further herein) may be coupled in an electrical path leading to the programmable resistors 152a,b of the MTIS 136 from defibrillators 1 202 and 2 204, and may be used in protecting the programmable resistors 152a,b and other components of the MTIS 136 from potential damage from delivered defibrillation shocks.

In some embodiments as described herein, an MTIS is used in providing resistance values to defibrillators that substitutes for, replaces, or mirrors, patient transthoracic impedance values that might otherwise be obtained by (non-blocked) defibrillator patient transthoracic impedance monitoring and measurements. Furthermore, in some embodiments, programmable digital resistors of the MTIS may be used in providing such mirror patient transthoracic impedance or resistance values to defibrillators.

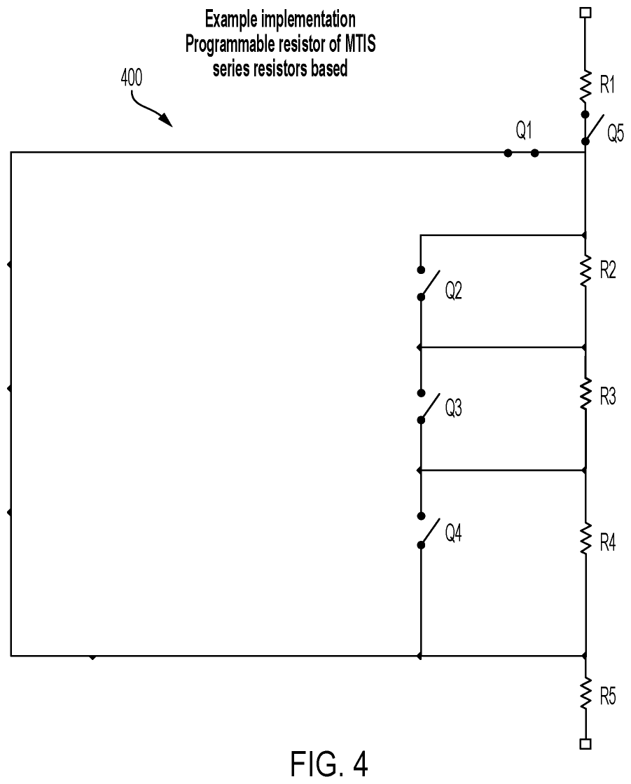

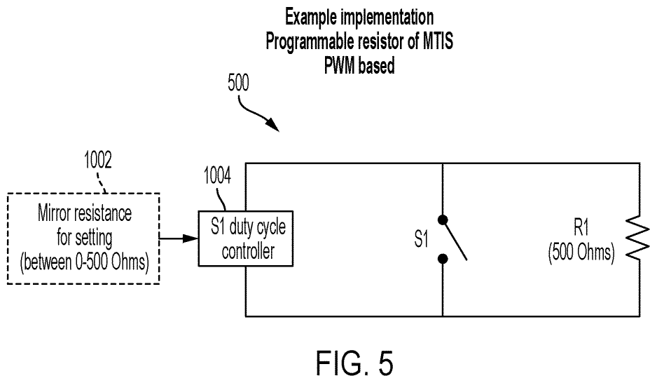

As such, in some embodiments, such as those illustrated in the examples of FIGS. 4 and 5, the programmable resistors 152a,b provide resistance values to the defibrillators 202, 204 (e.g. whether open loop or closed loop current control is used) that may mirror, or approximately mirror, the appropriate patient transthoracic impedance or resistance values that each of the defibrillators 202, 204 would otherwise measure in connection with the appropriate electrode pair 218, 220.

FIGS. 4 and 5 are example simplified circuit diagrams illustrating example programmable resistors 400, 500 of mirror transthoracic impedance monitoring subsystems, according to some embodiments. As depicted in FIG. 2D, the measurement circuit 156 inputs measured patient transthoracic resistance or impedance values (for each of the defibrillators 202, 204) to the interleaving timing control 154. Based on the input values, the interleaving timing control 154 determines or selects an appropriate programmable resistor resistance value for each of the programmable resistors 152a,b. Each of the programmable resistors 152a,b are then set or configured to reflect a corresponding resistance, such as a resistance that matches or nearly matches the measured value. In some embodiments, each of the programmable resistors 152a,b may be capable of being set to only a discrete set of resistance values. In such cases, in some embodiments, the programmable resistor 152a,b may be set to a resistance value that is closest to the appropriate measured value (however, in other embodiments, various other selection criteria may be used, such as by selecting the nearest lower or the nearest higher available resistance value to the measured value, or in other ways). Furthermore, in various embodiments, various components of the defibrillation accessory device system 242 may be used in determining or selecting the resistance values to which to set the programmable resistors 152a,b. For example, in some embodiments, the programmable resistors 152a,b themselves may include circuitry for such determination or selection.

As illustrated in FIG. 4, in some embodiments, either or each of the programmable resistors 152a,b may be based on a series of resistors, where an electrical circuit configuration is selected to generate and select an appropriate available resistance value. For example, in the circuit 400 shown in FIG. 4 as shown (which may be a simplified portion of a larger programmable resistor 152a,b), the circuit 400 includes a series of resistors R1-R5 as well as multiple switches Q1-Q4 arranged in parallel or series with each of the resistors R1-R5 (in the example shown, with Q1 being in series with R1 and R5, and Q2-Q4 being in parallel with resistors R2-R4, respectively). By selecting a configuration of each of the switches Q1-Q4 as open or closed, one of a set of available resistance values may be selected and generated. For example, if switch Q1 is set to a closed configuration (closed), as shown, then the resistance of the programmable resistor 152a,b will be equal to R1+R5, which is the minimum resistance value available. However, if Q1 is set to an open configuration (open), then the programmable resistor 152a,b will take on a resistance value that is dependent upon the particular configuration of each of the switches Q2-Q4. More specifically, if Q1 is open, then the resistance value will be equal to R1+R5 plus the resistance of any of resistors R2-R4 for which the corresponding switch Q2-Q4 is open (causing current to flow through the corresponding resistor) rather than closed (causing the corresponding resistor to be bypassed). Furthermore, if Q5 is set to an open configuration, then current will not flow through the circuit.

For example, if Q5 is set to an open configuration, then no current will flow through the circuit. Assuming that Q5 is set to a closed configuration, if Q1 and Q2 are open, and Q3 and Q4 are closed, then the programmable resistor 152a,b will take on a resistance value equal to R1+R2+R5. R2 will contribute to the total resistance value, because switch Q2 is open, causing current to pass through R2. However, R3 and R4 will not contribute, because switches Q3 and Q4 are closed, causing current to bypass, or short circuit, resistors R3 and R4. In the example shown, this can allow for a total of 9 possible and available specific resistance values (depending on the resistance values of each of the resistors), such as, e.g., Open Circuit (if Q5 is open), or, in Ohms, 25, 50, 75, 100, 125, 150, 175, 200, or other values depending on the values of each of R1-R5, or more or less values if more or less resistors are included.

Furthermore, in other embodiments, other circuit topologies may be used, in which particular circuit configurations, such as open or closed switches, can allow for various particular resistance values. In some embodiments, an available resistance value is chosen that is closest to an appropriate measured patient transthoracic impedance value, for example. If the interleaving timing control 154 toggles every half second, then, every half second, an updated measured patient transthoracic impedance value will be obtained and used to appropriately set each of the programmable resistors 152a,b to the appropriate resistance values. These will then be provided to each of the defibrillators 202, 204, so that one or more parameters for defibrillation shocks 1 and 2 can be determined accordingly.

As illustrated in FIG. 5, in some embodiments, each of the programmable resistors 152a,b (or either of them) may be implemented as pulse width modulation (PWM) based. FIG. 5 illustrates a simplified example of a PWM based programmable resistor 500 (or a portion thereof). As shown, a mirror resistance value for setting of the programmable resistor 500 is input to a controller 1004, which controls the duty cycle (for a cycle, the ratio of ON time to the complete cycle length, including ON time plus OFF time) of a switch, S1. For example, in some embodiments, the input mirror resistance setting value may be provided by the interleaving timing control 154 as shown in FIG. 2D, and may correspond with, or be based on, the appropriate measured patient transthoracic impedance value. The controller 1004 may then control the duty cycle of switch S1 to cause the resistance value of the programmable resistor 152a,b to be equal to, approximately equal to, or equal to the nearest available value to (or, e.g., the next highest or lowest available value to), or otherwise appropriately correspond with, the input mirror resistance setting value. Particularly, the inverse of the duty cycle ratio multiplied by the resistance value in parallel with the switch (in this example. R1, which is 500 Ohms), results in the resistance value for the programmable resistor 152a,b. For example, if the duty cycle is set to 0.5, then the resistance value for the programmable resistor 152a,b will be set to 250 Ohms. As such, various resistance values for the programmable resistor 152a,b may be available, between 0-500 Ohms, in this example, depending on the set duty cycle of switch S1, as set by controller 1004 based on the input mirror resistance value 1002.

It is noted that, in some embodiments that include use of a PWM based programmable resistor, other circuit adjustments may be required and made to the programmable resistor to ensure accuracy, or correct any inaccuracy, that might otherwise be present based on the overall operation of the circuit.

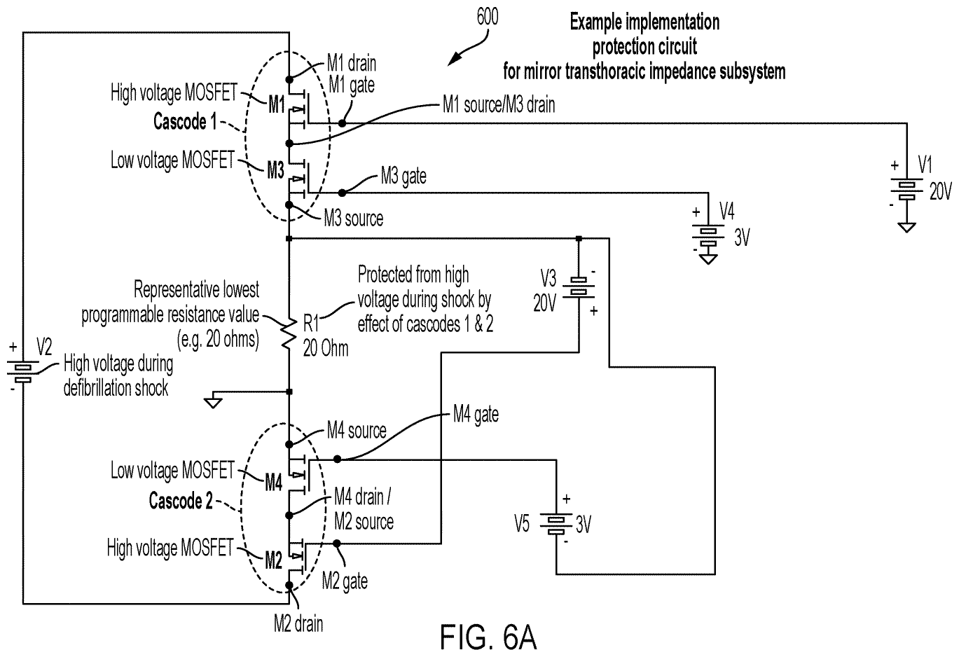

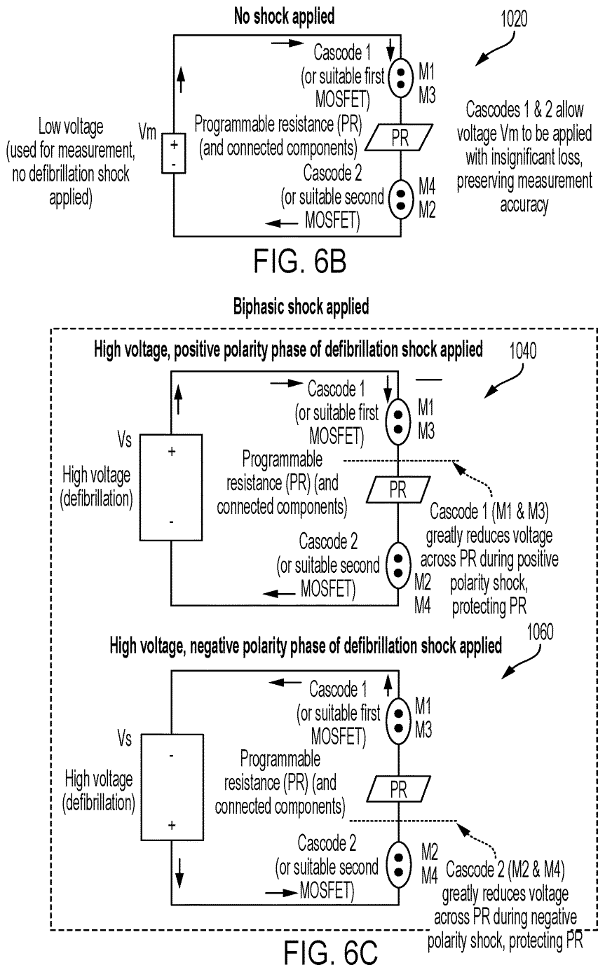

FIG. 6A is a simplified circuit diagram illustrating an example protection circuit 600 for a mirror transthoracic impedance subsystem. As described with reference to FIG. 2D, electrical protection circuits 138a,b may be coupled in an electrical path leading to the programmable resistors 152a,b of the MTIS 136 from defibrillators 1 202 and 2 204, and may be used in protecting the programmable resistors 152a,b and the MTIS 136 from potential damage from delivered defibrillation shocks. Protection circuit 600 is an example of, e.g., one of the electrical protection circuits 138a,b.

As described above, in some embodiments, in DSED using two defibrillators, programmable resistors (e.g., programmable resistors 152a,b as shown in FIG. 2D) may be used to provide resistance values to each of the defibrillators, e.g., to mirror the appropriate patient transthoracic impedance values. The programmable resistors are in electrical paths with shocks from the defibrillators and must be protected against potential damage from the high voltages of such shocks. The programmable resistors may. e.g., be digital resistors that include analog switch integrated circuits that are required to have low ON resistance and low capacitance. Such devices may typically only be capable of withstanding low voltages, such as, e.g., 5.5 volts, which is much lower than defibrillation voltages.

Defibrillator circuitry associated with high frequency impedance measurement by the defibrillator (which signaling may be blocked in some embodiments herein) may be protected against high voltage of defibrillation shocks by high voltage capacitors placed in series with the circuits to be protected. These protection capacitors must have a high enough capacitance to introduce insignificant or acceptably small high frequency impedance measurement error, and yet must also have low enough capacitance to result in insignificant or acceptably small reduction in delivered defibrillation shock energy. The defibrillator circuits to be protected may include the current source and the demodulator. The current source may consist of a relatively high voltage in series with a high source resistance. The protection capacitor must have an acceptably low impedance in comparison with the source resistance, so as to introduce insignificant or acceptably low measurement error. This may be achieved with a small capacitance value, as may be illustrated as follows.

$$Xc = 1 / ( 2 π fC ) \qquad ( Equation 1 )$$

where: Xc is capacitive reactance. π=the constant of approximately 3.14159, f is signal frequency, and C is capacitance.
In accordance with Equation 1, a high Xc allows for a low C, which allows a small capacitance value for the protective capacitor to protect the current source. The demodulator has a high input impedance, and so can also be protected with a small capacitance value for the protective capacitor.

However, in some embodiments, the programmable resistors, e.g. programmable resistors 138a,b as shown in FIG. 2D, may, in some instances or for some patient transthoracic impedances, be required to have a low resistance, such as a resistance as low as, e.g., 20 Ohms. Protection of such a low resistance value programmable resistor using a protective capacitor (for series protection) would require a large capacitance value. This large capacitance value may be required to be so large that it would divert an unacceptable amount of defibrillation shock energy away from the patient.

Besides series protection, another form of protection, such as may be used in surge protectors and other devices, is shunt protection. In shunt overvoltage protection, under appropriate circumstances, such as application of high voltage, a low resistance path is created for electrical current, to allow the current to pass around, e.g., a device or component to be protected. However, in various embodiments described herein, shunt protection is not suitable, because the DSED defibrillation shock must be delivered to the patient and not shunted back to the defibrillator. Therefore, series protection is required, and yet, series protection using a protective capacitor may also not be acceptable. As such, some embodiments provide a solution to the technical problem of providing acceptable electrical protection of programmable resistors, such as of an MTIS, such that sufficient protection is afforded without introducing an unacceptable amount of measurement error, and using series protection. FIGS. 6A-B and 7A-B provide examples of protection circuits, and operation thereof, that can be used in DSED to provide suitable series protection of, e.g., programmable resistors, such as programmable resistors 138a,b, even in implementations in which the programmable resistors 138a,b are required to have, or to sometimes have, low resistance values, such as, e.g., 20 Ohms. 10 Ohms or 5 Ohms.

In some embodiments, a protection circuit (for, e.g., a programmable resistor of an MTIS) is provided that uses multiple transistors, such as MOSFETs, in cascode configurations, which configuration may, e.g., allow control of the conduction state of a high voltage transistor or MOSFET using a low voltage MOSFET with a low voltage gate to source (Vgs) ON threshold voltage. As described in detail below, use of two cascodes in each protection circuit may allow use of readily available MOSFETs in the protection circuit.

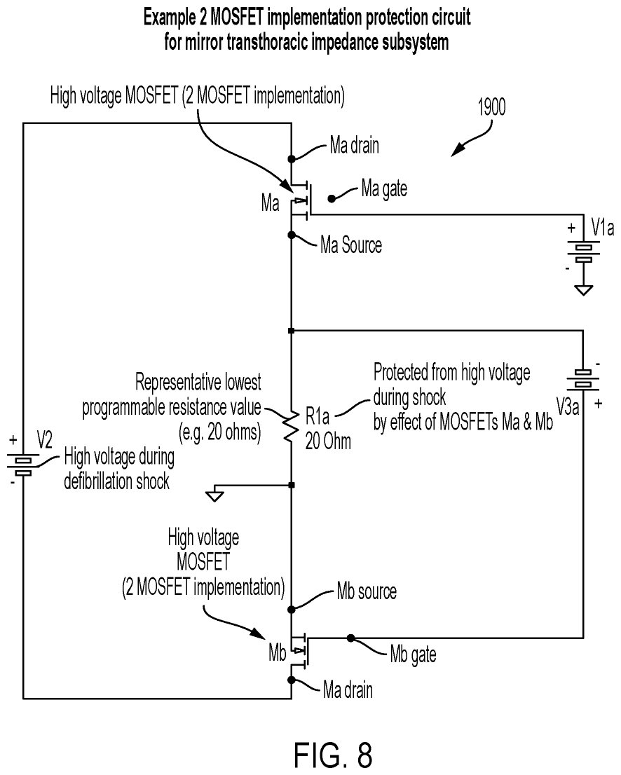

It is noted, however, that, as further described herein, in some embodiments, each protection circuit uses only two MOSFETS, instead of four, and no cascode configurations (as shown in FIG. 8, for example). In such an embodiment, a first suitable MOSFET may perform, or sufficiently perform, the function of cascode 1, including MOSFETs M1 and M3, and a second suitable MOSFET may perform, or sufficiently perform, the function of cascode 2, including MOSFETs M4 and M2. However, MOSFETs that fit the requirements for such embodiments may not be currently or readily available. For example, such MOSFETs may need to withstand much higher voltages than, e.g., 5.5 V.

FIG. 6A provides a simplified example protection circuit 600 (or a portion thereof) implemented accordingly. In the simplified protection circuit 600 shown in FIG. 6A, a representative lowest programmable resistance value of 20 ohms is shown as R1. A high voltage source V2 is shown, representing a voltage associated with a high voltage defibrillation shock, e.g. of DSED by a defibrillator (e.g., defibrillator 1 202 or 2 204 as shown in previous figures). Two cascodes, cascodes 1 and 2, are also shown, with cascode 1 including MOSFETs M3 (low voltage) and M1 (high voltage) and cascode 2 including MOSFETs M4 (low voltage) and M2 (high voltage), with each of the MOSFETs having terminals of drain, gate and source labelled accordingly. Associated voltage sources of V1 (20V), V3 (3V), V4 (20V) and V5 (3V) are also shown. Cascodes 1 and 2 operate to protect the programmable resistor, even set to a lowest resistance (represented by R1 of 20 Ohms), including during both phases of. e.g., a biphasic defibrillation shock (or a monophasic shock).

It is noted that, as described further below, in some embodiments, cascode 1 may be replaced with a single MOSFET, and/or cascode 2 may also be replaced with a single MOSFET, but each of the two MOSFETs would need to have suitable characteristics. However, MOSFETs that fit the requirements for such embodiments may not be currently or readily available.

FIGS. 6B and 6C provide conceptual illustrations of aspects of operation of the example protection circuit of FIG. 6A. More specifically, image 1020 of FIG. 6B illustrates operation when no defibrillation shock is applied, but low voltage monitoring and measurement voltage of Vm is applied. FIG. 6B illustrates operation when a biphasic defibrillation shock of voltage Vs is applied, with image 1040 relating to operation during the positive phase of the biphasic shock and image 1060 relating to operation during the negative phase of the biphasic shock.

In image 1020 of FIG. 6B, Vm represents an applied low voltage source used in, e.g., monitoring and measurement of patient transthoracic impedance, such as by a MTIS. Cascodes 1 and 2 are also shown, including MOSFETS M1 and M3, and M4 and M2, respectively. Box PR is shown, representing a programmable resistor and potentially other connected circuit components, coupled between the two cascodes. In operation, cascodes 1 and 2 allow voltage Vm to be applied with insignificant or acceptably small loss, preserving sufficient measurement accuracy, using series protection. Although, in FIG. 6B, current is shown flowing in a clockwise or positive direction, in some embodiments, the direction of current flow alternates from a clockwise or positive direction to a counterclockwise or negative direction.

More particularly, MOSFET M3 is optimized for low ON state resistance and low Gate to Source threshold voltage. As shown, M3 has a gate voltage supplied by V4 of 3V. This gate voltage exceeds the Vgs ON threshold by a sufficient margin to result in the conduction state of M3 being low resistance. MOSFET 4 has a gate voltage supplied by V1 of 20 V. This gate voltage exceeds the Vgs ON threshold by a sufficient margin to result in the conduction state of M4 being low resistance. Similar factors apply to MOSFETs M2 and M1. As such, all four MOSFETs are in a low resistance conduction state, and so the voltage Vm is applied across PR with insignificant or acceptably small loss, thus preserving sufficient measurement accuracy, using series protection.

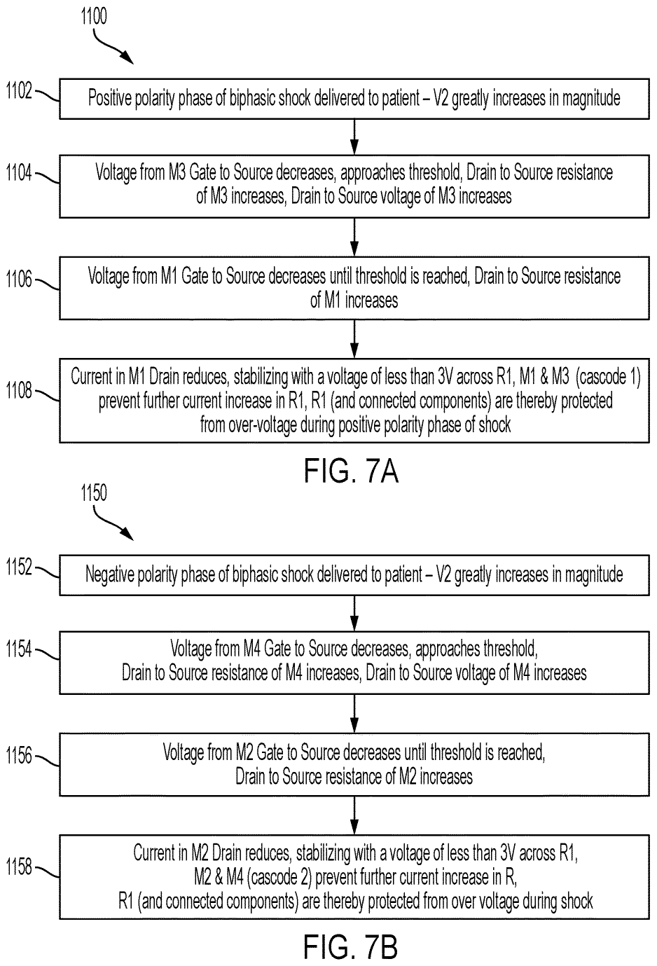

In image 1040 of FIG. 6C, the positive phase of a biphasic defibrillation shock is applied, with the shock voltage source represented as Vs. As shown in FIG. 6C, and as further illustrated with reference to FIGS. 7A-B, cascode 1, including MOSFETs M1 and M3, operates to protect PR by greatly reducing the voltage across PR during the positive phase of the biphasic shock, where defibrillation current passes across cascode 1 before reaching PR.

In image 1060 of FIG. 6C, the negative phase of the biphasic defibrillation shock is applied (immediately following the positive phase), with the shock voltage source again represented as Vs. As shown in FIG. 6C, and as further illustrated with reference to FIGS. 7A-B, cascode 2, including MOSFETs M4 and M2, operates to protect PR by greatly reducing the voltage across PR during the negative phase of the biphasic shock, where defibrillation current passes across cascode 2 before reaching PR.

FIGS. 7A and 7B provide flow diagrams 1100, 1150 that illustrate operation of the example protection circuit illustrated in FIG. 6A, where the protection circuit uses four MOSFETS in two cascode configurations. As noted above, in some embodiments, example protection circuits may use only two MOSFETS instead of four, and may not require any cascode configurations. However, MOSFETs fitting the requirements for this may not be readily or currently available. In some embodiments, examples using four MOSFETS in two cascode configurations provide a solution to technical problems created the limitations of readily or currently available MOSFETs. Moreover, in various embodiments, both the four MOSFET and two MOSFET (if suitable MOSFETs are available) example protection circuits provide solutions to the technical problem of providing suitable protection for the programmable resistor circuits.

More particularly, in some embodiments, readily or currently available high voltage MOSFETS may have Gate to Source threshold voltages that are too high for the analog switches of the programmable resistor circuits. However, readily or currently available low voltage MOSFETS with low Gate to Source thresholds have Drain to Source voltage ratings that are much too low to protect against the high voltage of defibrillation shocks. As such, in some embodiments of a protection circuit, two cascodes are included, where each cascode includes both a high voltage MOSFET and a low voltage MOSFET, where the high voltage MOSFET facilitates providing sufficient high voltage protection but have threshold voltages that are too high for the analog switches of the programmable resistor circuit, and where the low voltage MOSFET addresses this problem by having sufficiently low Gate to Source thresholds.

More specifically, in the examples illustrated in FIGS. 6A-C, which use four MOSFETs per protection circuit, suitable low voltage MOSFETs (e.g., meeting the requirements of M3 and M4) are currently and readily available, but, as mentioned above, have Drain to Source voltage ratings that are much too low to protect against the high voltages of defibrillation shocks. Suitable High voltage MOSFETs (e.g., meeting the requirements of M1 and M2) are relatively recent developments in the semiconductor market, and may be available to incorporate in embodiments of the present disclosure.

In FIG. 7A, flow diagram 1100 illustrates operation of the protection circuit 600 of FIGS. 6A (including two cascodes of two MOSFETs each). More particularly, flow diagram 1100 relates to circuit operation as conceptually illustrated in FIG. 6B, during application of the positive phase of a biphasic defibrillation shock. At step 1102, the positive polarity phase of the high voltage shock is delivered to the patient, causing the applied voltage source to become large in magnitude. At step 1104, the voltage from M3 Gate to Source decreases and approaches the threshold, the Drain to Source resistance of M3 increases, and the Drain to Source voltage of M3 increases. At step 1106, the voltage from M1 Gate to Source decreases until the threshold is reached, and the Drain to Source resistance of M1 increases. Finally, at step 1108, the current in the M1 Drain reduces, stabilizing with a voltage of less than 3V across R1. As such, M1 and M3 (cascode 1) prevent further current increase in R1, and R1 (and connected circuit components) are thereby protected from over-voltage during the positive polarity phase of the shock.

As such, during the positive polarity phase of the shock, cascode 1 provides protection. Cascode 2 (M4 and M2) does not, but current flows unimpeded in the body diodes of M4 and M3 of cascode 2, which diodes are an intrinsic part of the MOSFET construction. Conversely, during the negative phase of the shock, as described below, cascode 2 (M4 and M2) provides protection, while cascode 1 (M1 and M3) does not, but current flows unimpeded through M4 and M3 of cascode 1. As such, the protection circuit may be implemented in an intentionally symmetric fashion in this regard. It is noted that, in other embodiments, assuming that high voltage MOSFETs are available that meet all requirements for a two MOSFET only embodiment of a protection circuit, as described above, then cascode 1 and cascode 2 would each be replaced by a single suitable high voltage MOSFET, such that only two MOSFETs would be required for the protection circuit, and no cascode configurations would be required.

In FIG. 7B, flow diagram 1150 relates to circuit operation as conceptually illustrated in FIG. 6C, during application of the negative phase of a biphasic defibrillation shock (e.g., immediately following the positive phase). At step 1152, the negative polarity phase of the high voltage shock is delivered to the patient, causing the applied voltage source to become large in magnitude. At step 1154, the voltage from M4 Gate to Source decreases and approaches the threshold, the Drain to Source resistance of M4 increases, and the Drain to Source voltage of M4 increases. At step 1156, the voltage from M2 Gate to Source decreases until the threshold is reached, and the Drain to Source resistance of M2 increases. Finally, at step 1158, the current in the M2 Drain reduces, stabilizing with a voltage of less than 3V across R1. As such, M2 and M4 (cascode 2) prevent further current increase in R1, and R1 (and, e.g., connected circuit components) are thereby protected from over-voltage during the negative polarity phase of the shock. During the negative phase of the shock, cascode 2 (M4 and M4) provide protection, while cascode 1 (M1 and M3) does not, but current flows unimpeded through M1 and M3 of cascode 1.

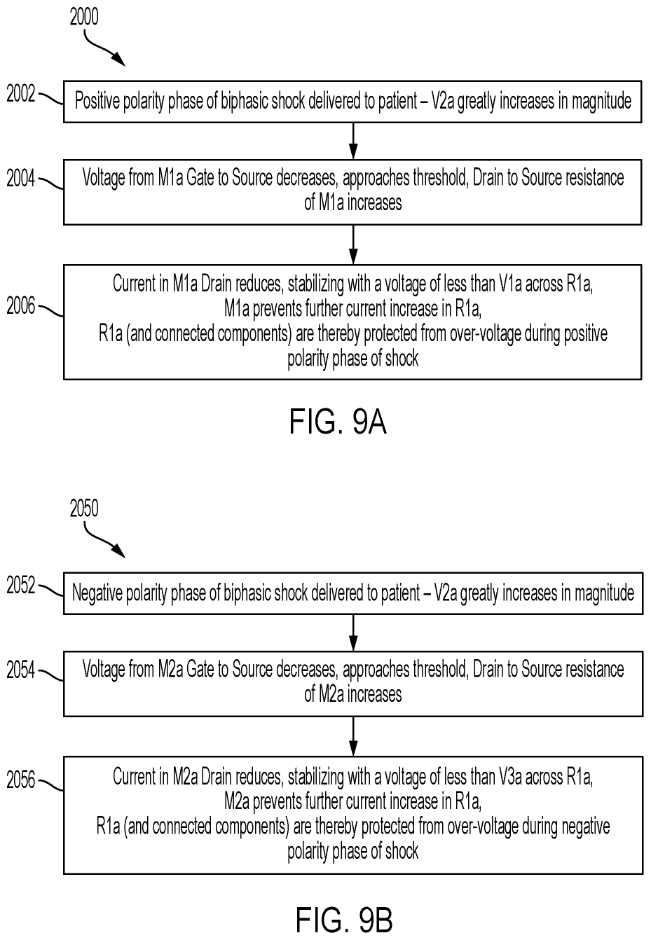

FIG. 8 is a simplified circuit diagram 1900 illustrating an example protection circuit for a mirror transthoracic impedance subsystem, using a implementation with 2 high voltage MOSFETs. As described above, in a 2 MOSFET implementation, each of the 2 MOSFETs, if available, may be required to be rated to withstand voltages much higher than, e.g., 5.5 V. In the example depicted in FIG. 8, the 2 MOSFETs are MOSFETs Ma and Mb, with the respective drain, gate and source for each of the 2 MOSFETs labelled in FIG. 8. As depicted, voltages V1a and V3a define the maximum voltage stress that may be applied to resistance R1a, which is, in the example shown, 20 ohms, which represents an example lowest programmable resistance value. V2a represents high voltage applied during a defibrillation shock. As shown, relative to the implementation shown in FIG. 6A, MOSFET Ma accomplishes the function of cascode 1, and MOSFET Mb accomplished the function of cascode 2, as further described with reference to FIGS. 9A and 9B.

FIGS. 9A and 9B are flow diagrams 2000, 2050 illustrating example operation of the protection circuit of FIG. 8, with flow diagram 2000 relating to operation during a positive polarity phase of a delivered biphasic defibrillation shock, and flow diagram 2050 relating to operation during a negative polarity phase of the delivered biphasic defibrillation shock.

In flow diagram 2000 of FIG. 9A, at step 2002, the positive polarity phase of the high voltage shock is delivered to the patient, causing the applied voltage source to become large in magnitude. At step 2004, the voltage from M1a Gate to Source decreases and approaches the threshold, the Drain to Source resistance of M1a increases. At step 2006, the current in Mia Drain reduces, stabilizing with a voltage of less than V1a across R1a, M1a prevents further current increase in R1a, and R1a (and connected components) are thereby protected from over-voltage during the positive polarity phase of the shock.

In flow diagram 2050 of FIG. 9B, at step 2052, the negative polarity phase of the high voltage shock is delivered to the patient, causing the applied voltage source to become large in magnitude. At step 2054, the voltage from M2a Gate to Source decreases and approaches the threshold, the Drain to Source resistance of M2a increases. At step 2056, the current in M2a Drain reduces, stabilizing with a voltage of less than V3a across R1a, M2a prevents further current increase in R1a, and R1a (and connected components) are thereby protected from over-voltage during the negative polarity phase of the shock.

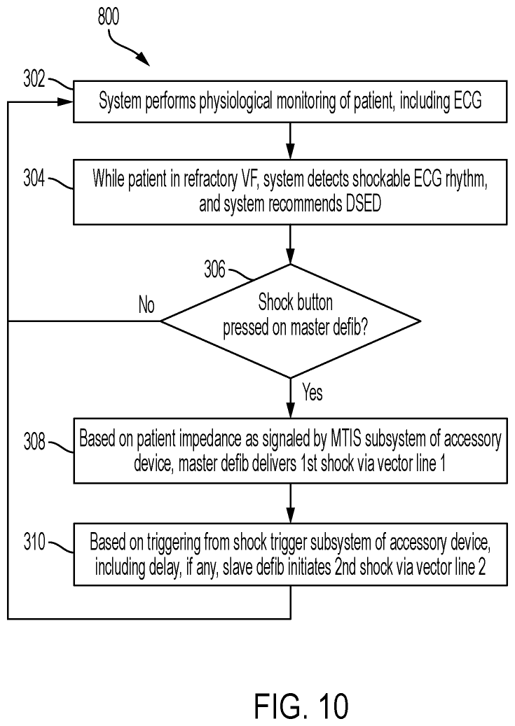

FIG. 10 is a simplified flow diagram 800 relating to an example embodiment of DSED as implemented on a DSED system such as the DSED system depicted in FIGS. 2A-B. At step 302, the DSED system performs physiological monitoring of the patient, including ECG monitoring. At step 304, while the patient is in refractory VF, the system detects a shockable ECG rhythm, and the system provides a recommendation to the user to cause DSED to be delivered to the patient. At step 306, if the user has not pressed the shock button (or DSED shock button) (e.g. on the master defibrillator, such as defibrillator 1 in previous figures), then the method 800 returns to step 302. However, at step 306, if the user presses the DSED shock button on the master defibrillator (or otherwise selects to immediately implement DSED, whether the selection is made on the master defibrillator or another device or interface), then the method 800 proceeds to step 308. At step 308, based at least in part on patient impedance as signaled by the MTIS subsystem of the accessory device of the DSED system, the master defibrillator delivers the first shock to the patient via vector line 1 (e.g., via a first pair of electrodes attached to the accessory device). Next, at step 310, based on triggering from the shock trigger subsystem of the accessory device, including any delay of a shock delay period, the slave defibrillator (e.g., defibrillator 2 of previous figures) delivers the second shock via vector line 2, which is different than vector line 1 (e.g., via a second pair of electrodes attached to the accessory device). Following step 310 (after DSED has been delivered to the patient), the method returns to step 302. It is noted that, in some embodiments, as described herein, a DSED shock button, which may cause delivery of both DSED shocks, may be included on the defibrillation accessory device.

FIGS. 11A and 11B illustrate an example DSED system 900 including two defibrillators, defibrillator 1 402, which may be the master defibrillator, and defibrillator 2 404, which may be the slave defibrillator. Unlike the DSED system 200 illustrated in FIG. 2A-B, this system 900 does not include or require a DSED accessory device for DSED to be properly administered in accordance with the present disclosure. As shown, electrodes 406a,b (in anterior and lateral positions on the patient) of electrode pair 1 are connected with defibrillator 1 402 and electrodes 408a,b (in anterior and posterior positions on the patient) of electrode pair 2 are connected with defibrillator 2 404. Each of the defibrillators 402, 404 includes a DSED system 450, 452. In various embodiments, the defibrillators 402, 404 are communicatively connected, such as by wired and/or wireless connection (or both, where one can replace the other if/as needed).

Each of the defibrillators 402, 404 each include their own inter-defibrillator communication subsystem 442, 446 to allow the defibrillators 402, 404 to communicate with each other as required and described herein for DSED to be properly administered.

Defibrillator 1 402 includes a defibrillator 1 shock trigger subsystem 438, a defibrillator 1 impedance monitoring subsystem 440 and a defibrillator 1 inter-defibrillator communication subsystem 442. Defibrillator 2 404 includes a defibrillator 2 shock trigger subsystem 439, a defibrillator 2 impedance monitoring subsystem 444 and a defibrillator 2 inter-defibrillator communication subsystem 446.

As depicted, defibrillator 1 402 is, or is functioning in this embodiment as, the master defibrillator and defibrillator 2 404 is, or is functioning in this embodiment as, the slave defibrillator. In some embodiments, defibrillator 1 402 is implemented to deliver DSED shock 1 (the first shock), such as upon selection by a user, e.g., pressing of a DSED button on defibrillator 1 402. Defibrillator 2 404 is implemented to deliver DSED shock 2 (the second shock), after a shock delay period following the delivery of shock 1 (e.g., 0-75 milliseconds). The defibrillator 1 402 shock trigger subsystem 438 may include circuitry and software to enable delivery of shock 1 upon selection by the user and to communicate to defibrillator 2 404 when such delivery is implemented. The defibrillator 2 404 shock trigger subsystem 439 may include circuitry and software to enable delivery of shock 2 after a shock delay period following shock 1. In some embodiments, the communication by defibrillator 1 402 to defibrillator 2 404 that delivery of shock 1 is occurring provides a trigger to defibrillator 2 404 to deliver shock 2 after the shock delay period following delivery of shock 1.

It is noted that, in various embodiments, circuitry and software of each of the defibrillators and for each of the subsystems may be divided in various varies between them, and the depicted embodiment is one example thereof. Furthermore, in some embodiments, for all of the embodiments described herein including more than one defibrillator, each of the defibrillators may be implemented to be capable of functioning as the master or the slave defibrillator, and may have all necessary subsystems for such roles, whereas, in other embodiments, each of the defibrillators is implemented only to function as the master or slave defibrillator.

In some embodiments, defibrillator 1 402 delivers shock 1 immediately upon a corresponding selection/command by the user. However, in other embodiments, as described further as follows, defibrillator 1 402 may be capable of being preset with a time of delivery of shock 1, such that, after a particular delay period, shock 1 is delivered, where the user is aware of, and/or sets, the particular delay period. Such a delay period may, e.g., allow ensuring that DSED shocks are delivered with the required shock delay period between them, even in the event of interrupted communication between them, for example.

In some embodiments, given that wireless and even wired communication can sometimes fail or be subject to unpredictable intermittent interruptions, one or both of the shock trigger subsystems 438, 439 may be implemented to allow DSED to occur as required even in the event of a communication interruption or failure, or, in the event that is not safely and reliably possible, to cancel otherwise anticipated DSED shock delivery. For example, in some embodiments, one or both of the defibrillators 402, 404 are implemented to include a precise timer/clock that can be set to a particular delay period (e.g., a fraction of a millisecond, one or more milliseconds, a fraction of second, or one or more seconds), where the DSED defibrillation shocks are delivered following the particular delay period (and, potentially, for shock 2, after a shock delay period following delivery of shock 1). That is, in some embodiments, shock 1 may be delivered after the particular delay period following selection by a user, and shock 2 may be delivered after the particular delay period and after a shock delay period following delivery of shock 1.

For example, Defibrillator 2 404 may be set such that, after receiving a communication from defibrillator 1 402 that shock 1 is set to be delivered at a particular time (after the particular delay period), shock 2 is delivered following the particular delay period and the shock delay period, such that shock 2 is delivered at the correct time following shock 1 (which may or may include an extremely small about of time (e.g., less than 1 millisecond) to initiate delivery, as described previously herein). Once defibrillator 1 402 is set to deliver shock 1 and communicates accordingly with defibrillator 2, 404 (where, in some embodiments, any communication between the defibrillators may include. e.g., confirmations of receipt of communications, etc.), both defibrillators may be implemented to proceed with their respective DSED shocks at the set times even if communication between them is interrupted before and/or through delivery. This may allow DSED to reliably occur on schedule even if communication between the defibrillators is interrupted.

Furthermore, in some embodiments, one or both of the defibrillators 402, 404 may be implemented so that steps are taken (e.g., required communicated checking or confirmations between the defibrillators 402, 404, cancellations, alarms, etc.) any time communication is interrupted or interrupted during particular circumstances, or interrupted for at least a threshold period of time, or interrupted for at least a threshold period of time dependent on circumstances, etc.

In some embodiments, one or more failsafe systems may be used to prevent potential failure, for example, in the event that defibrillator 1 402 is set to delivery shock 1 but communication to defibrillator 2 to communicate the set timing to delivery shock 1 fails. For example, in some embodiments, defibrillator 1 402 may be implemented to require communicated confirmation from defibrillator 2 404 that the communication has been received, otherwise defibrillator 1 402 may cancel set delivery of shock 1 and, e.g., notify the user accordingly. For example, in some embodiments, if, during the particular delay period, either defibrillator becomes disabled or patient monitoring leads to a determination that DSED must be cancelled, each defibrillator may be implemented to communicate this, which may cause cancellation by both defibrillators, or communication during the particular delay period and nearer to the shock times may be required for confirmation, or the user may be alerted and required to perform confirmation or some action, etc.

As shown in FIG. 11B, each of the defibrillators 402, 404 includes an impedance monitoring subsystem 442, 444. In some embodiments, each of the defibrillators 402, 404 is implemented so as not to continuously monitor for patient transthoracic impedance. Rather, the impedance monitoring subsystems 442, 444 may be implemented, and may operate in communication and coordination, such that alternating impedance monitoring is performed by each of the defibrillators 402, 404, in a manner that prevents simultaneous monitoring and may, e.g., be analogous to the alternating impedance monitoring (alternating, e.g., every fraction of a second, half second, or several seconds) performed by the accessory device 210 of FIG. 2A-D. The defibrillators 402, 404 may be implemented to communicate with each other in this regard, and timing of future alternating impedance monitoring may be coordinated, for example, slightly ahead of time, and may be coordinated using timers or clocks. Furthermore, communication failsafe systems may be used, analogously to the measures that may be taken in connection with ensuring timely and coordinated delivery of DSED shocks even in the event of communication failure, or cancellation, where appropriate. For example, in some embodiments, if lack of communication may jeopardize properly timed alternating impedance monitoring by one or both of the defibrillators 402, 404, one or both of the defibrillators 402, 404 may be implemented to cancel DSED, cancel the ability to select DSED, trigger confirmations, alarms or notifications, etc.

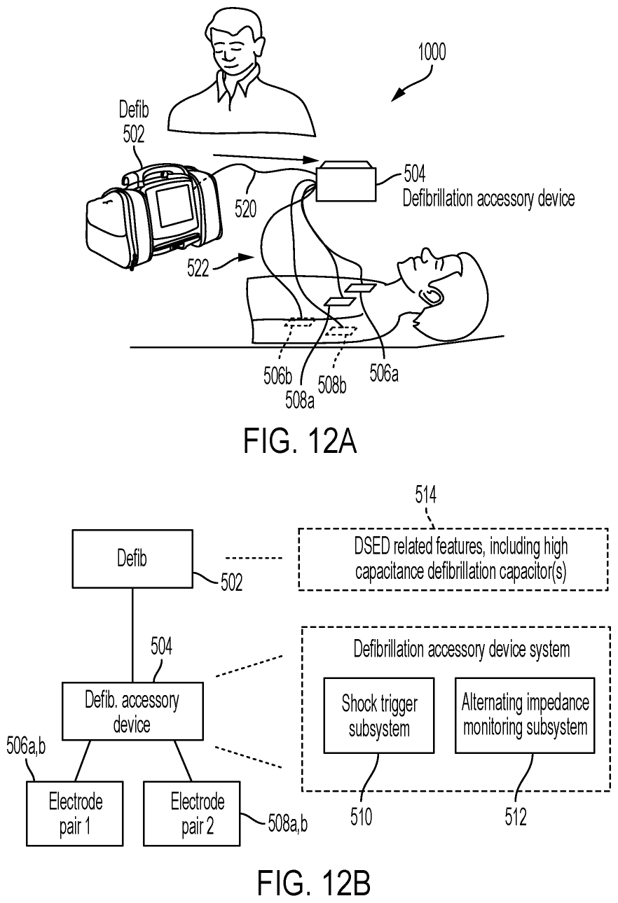

FIGS. 12A and 12B illustrate an example DSED system 1000 including one defibrillator 502 and a defibrillation accessory device 504. In this embodiment, the defibrillator 502 may be cable connected to the accessory device 504, and two pairs of electrodes are also connected to the accessory device 504 (electrode pair 1 including electrodes 506a,b positioned in anterior and lateral positions on the patient, and electrode pair 2 including electrodes 508a,b positioned in anterior and posterior positions on the patient), such that either pair of electrodes can be placed in electrical connection with the defibrillator 502, such as via the accessory device 504. In this embodiment, the single DSED defibrillator 502 has particular features 514 to allow it to implement DSED, including, for example, a high capacitance capacitor (or several capacitors) to provide sufficient capacitance for shock 1 and shock 2 without recharging. The defibrillator 502 may have other DSED related features as well, such a communication features or a communication subsystem to facilitate communication and coordination with the accessory device 504, DSED related failsafe systems, or features related to interaction with the user (e.g., a DSED button or control, DSED related notifications, alarms, etc.), although, in other embodiments, such features may be included in an accessory device, or distributed between a defibrillator and an accessory device, for example.

As depicted in FIG. 12A, the defibrillator 502 is connected by cable 520 to the defibrillation accessory device 504. Furthermore, cables lead from the defibrillation accessory device 504 to each of electrodes 506a-b and 508a-b, via one or more ports 524 (or, e.g., a single port with multiple inputs). For example, in various embodiments, all four of the electrodes 506a-b, 508a-b may connect by cable to the defibrillation accessory device 504 via a single port, or each of the two pairs may connect via one of two ports, or each of the four electrodes may connect via one or four ports.

The accessory device 504 may include a DSED shock trigger subsystem 510 implemented to sense delivery of shock 1 and trigger delivery of shock 2 following a shock delay period. In some embodiments, the shock trigger subsystem 510 may also facilitate electrically connecting electrode pair 1 with the defibrillator for delivery of shock 1, and then immediately switching to electrically connecting of electrode pair 2 with the defibrillator for delivery of shock 2, or the defibrillator may have this role, or it may distributed between both devices, for example.

The accessory device 504 may also include an alternating impedance monitoring subsystem 512 which may enable alternating patient transthoracic impedance monitoring, associated with each of the electrode pairs 1 and 2, by the defibrillator 502. For example, the alternating impedance monitoring subsystem 512 may alternate between electrically connecting electrode pair 1 and electrode pair 2 with the defibrillator 502, to facilitate such alternating monitoring. As such, in some embodiments, the defibrillator may be implemented to perform continuous impedance monitoring, but, via the action of the accessory device 504, alternating impedance monitoring between the electrode pairs 1 and 2 may be accomplished.

FIGS. 13A and 13B illustrate an example DSED system 1100 including one defibrillator 602. Connected with the defibrillator 602 are electrode pairs 1 and 2 (including electrodes 606a,b positioned in anterior and lateral positions on the patient and electrodes 608a,b positioned in anterior and posterior positions on the patient).

The defibrillator 602 may have various features to enable DSED, including high capacitance (e.g., as described with regard to the embodiment of FIGS. 10A and 10B). The defibrillator 602 may also include a shock trigger subsystem 652 and an alternating impedance monitoring subsystem 654, which subsystems 652, 654 may operate analogously to the corresponding subsystems of the accessory device 504 as described with reference to FIGS. 10A-B, but be adapted for operation with the single defibrillator 602, or may be otherwise implemented.

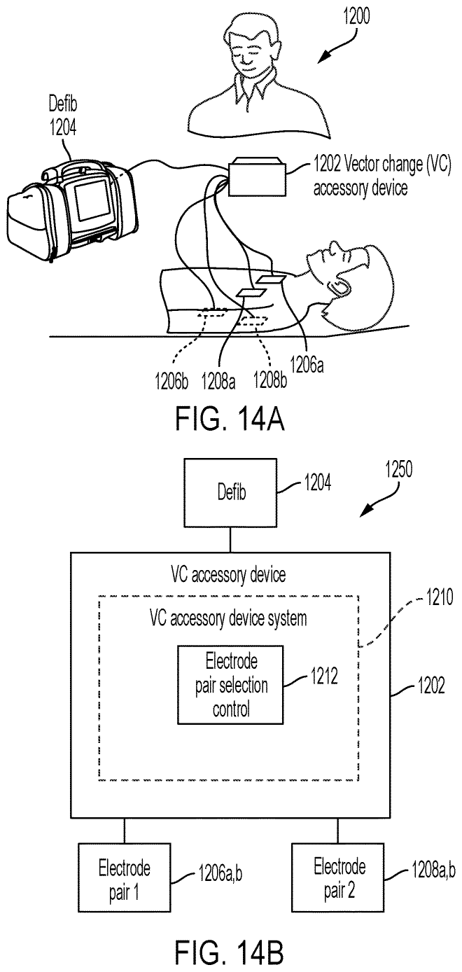

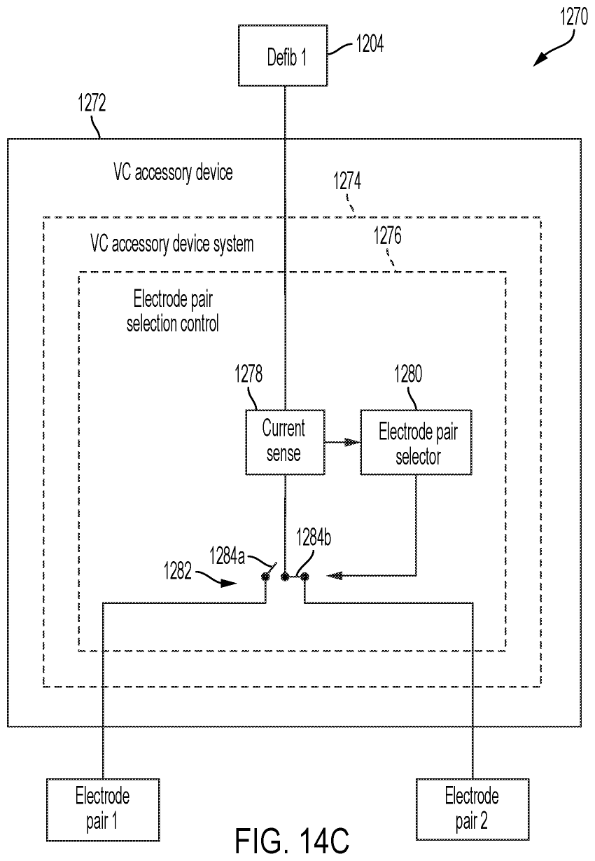

FIGS. 14A and 14B illustrate an example of defibrillation with vector change, including use of a defibrillator 1204 and a vector change accessory device 1202. In some embodiments, defibrillation with vector change may include delivery of two (or more) defibrillation shocks, where, e.g., the second shock is delivered via a different vector line than the first shock, such as by being delivered by a different electrode pair than the first shock. As depicted, the first shock may be delivered along a vector line defined by electrode pair 1, including electrodes 1206a,b, positioned in anterior and lateral positions on the patient, and the second shock may be delivered along a different vector line defined by electrode pair 2, including electrodes 1208a,b, positioned in anterior and posterior positions on the patient. In some embodiments of defibrillation with vector change, unlike some embodiments of DSED, the second shock may be performed after a much longer period of time than in DSED (e.g., seconds or minutes), and the second shock may not be, and may not need to be, delivered after a specified period of time relative to the first shock. For example, the second shock might be delivered after a period of CPR chest compressions, or after a period of chest compressions followed by ventilations (e.g., with a 30:2 protocol, after an approximately 2 minute period including 30 chest compressions followed by 2 ventilation breaths).

It is to be noted that, in other embodiments, as with DSED, defibrillation with vector change may be implemented without an accessory device, or with multiple defibrillators.

As depicted, the vector change accessory device 1202 may include a vector change accessory device system 1210, which may include an electrode pair selection control 1212, among other things. The electrode pair selection control 1212 may be used to select the electrode pair, of electrode pairs 1 and 2, to be used for a particular defibrillation shock, and may facilitate electrical connection of the appropriate pair of electrodes (electrode pair 1 or 2) with the defibrillator 1204 accordingly. For example, electrode pair 1 may be electrically connected with the defibrillator 1204 for delivery of the first shock, and electrode pair 2 may be electrically connected with the defibrillator 1204 for delivery of the second shock, for example.

FIG. 14C is a simplified circuit diagram 1270 illustrating a vector change accessory device 1272, such as may be an example of the vector change accessory device 1202 shown in FIGS. 14A-B. As depicted, the vector change accessory device 1272 includes a vector change accessory device system 1274, which includes an electrode pair selection control 1276 including a current sense 1278 and an electrode pair selector 1280. The current sense 1278 may be implemented, for example, using a current sense transformer. The current sense 1278 may be used to detect delivery of a shock (e.g., the first shock) along a particular vector line (e.g., using electrode pair 1). The current sense 1278 may signal the electrode pair selector 1280 accordingly. The electrode pair selector may then control the configuration of a one or more switches or switch network 1282 in order to select, and potentially also to electrically connect, the appropriate electrode pair (e.g., in order to switch from electrode pair 1 to electrode pair 2) for delivery of the subsequent shock (e.g., shock 2) along a different vector line than shock 1. As shown, the switch network includes two switches, where switch 1 1284a is open and switch 2 1284b is closed, causing connection of electrode pair 2, such as for delivery of the second next shock.

In various embodiments, the electrode pair(s) selected by the electrode pair selection control 1276 may be selected in various ways, e.g., by being preset by a user, preset in the device 1272 or its software, or selected by the electrode pair selection control 1276 based on particular selection criteria or using one or more selection algorithms, for example.

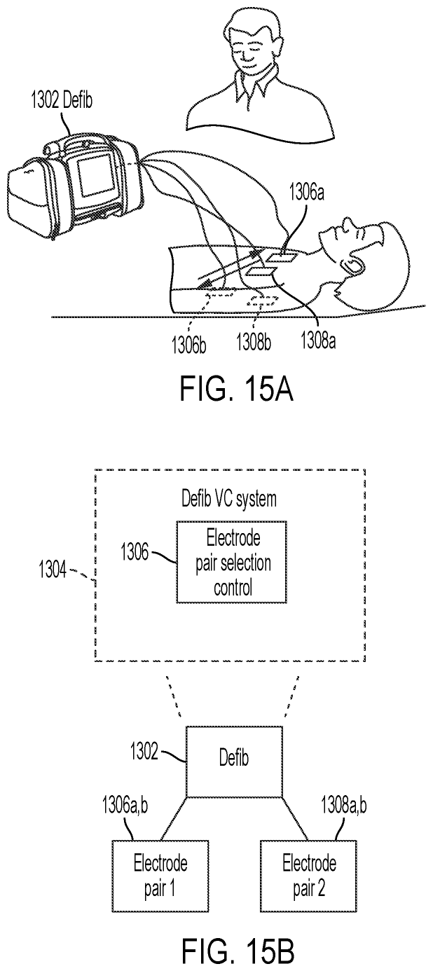

FIGS. 15A and 15B illustrate an example of defibrillation with vector change, including a defibrillator 1302 with a defibrillator vector change system 1304 including an electrode pair selection control 1306. As depicted, electrode pair selection control 1306 may control the appropriate pair of electrodes (electrode pair 1 or 2) in order to cause. e.g., a second shock to be delivered along a different vector line than the first shock (e.g., by switching from pair 1, used for delivery the first shock, to pair 2, to be used for delivery of the second shock). As depicted, electrode pair 1 includes electrodes 1306a,b, positioned in anterior and lateral positions on the patient, and electrode pair 2 includes electrodes 1308 a,b, positioned in anterior and posterior positions on the patient.

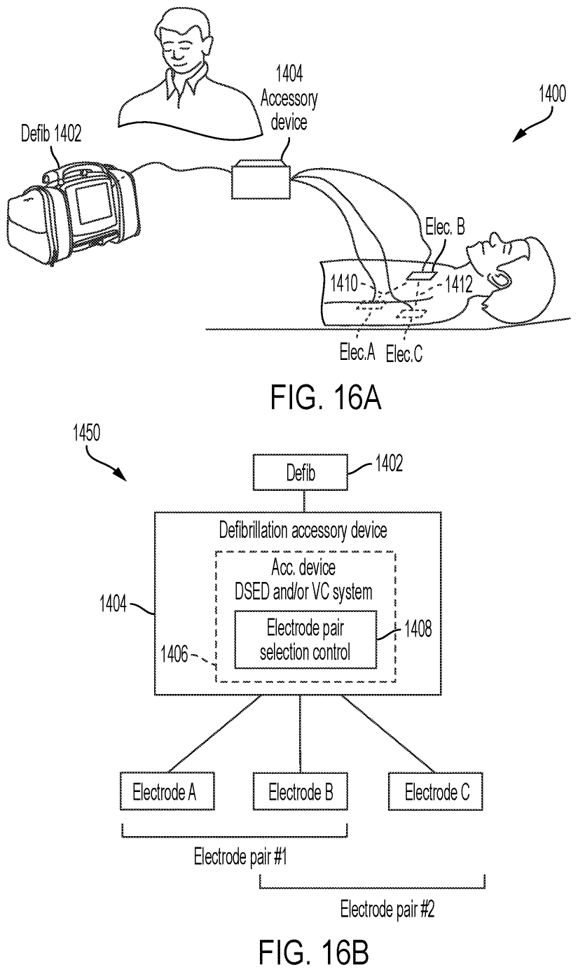

FIGS. 16A and 16B illustrate an example of DSED or defibrillation with vector change including a defibrillator 1402 and a defibrillation accessory device 1404, and including use of three electrodes, including electrodes A, B and C, which are connected to the accessory device 1404, such as via one or more ports. As shown, electrode B is positioned in an anterior position on the patient, electrode A is positioned in a lateral position, and electrode C is positioned in a posterior position. Electrodes A, B and C are connected with the accessory device 1404, such as via one or more ports.

As shown, the defibrillation accessory device 1404 includes an accessory device DSED system (or an accessory device vector change system) 1406, which includes an electrode pair selection control 1408 (which may differ depending on whether the system is for DSED or defibrillation with vector change, or may include multiple modes of operation to accommodate either DSED or defibrillation with vector change).

The electrode pair selection control 1408 may be implemented and operate similarly to an electrode pair selection controls as previously described for DSED or defibrillation with vector change using an accessory device. However, in the embodiment shown, electrode pair 1 includes electrodes B and A, and electrode pair 2 includes electrodes B and C, so that electrode B is included in both pairs. As a result, electrode pair 1 defines vector line 1410 (anterior-lateral), and electrode pair 2 defines different vector line 2 1412 (anterior-posterior), where the vector lines 1 and 2 are defined including use of one common electrode—electrode B.

As such, in the embodiment of FIGS. 16A-B, DSED or defibrillation with vector change can be implemented using only three electrodes to form two electrode pairs and two different vector lines. As shown, the common electrode is electrode B. However, in other embodiments, other electrodes may serve as the common electrode (e.g., in this example, electrodes A or B). Additionally, in some multielectrode pad embodiments, as described herein, the common electrode may be any electrode of a multielectrode pad.

In some embodiments, use of a defibrillation accessory device may allow use of three electrodes (e.g., to form two pairs of electrodes), or multielectrode pads, even with older defibrillators or defibrillators that are otherwise not implemented with features for such use. In some embodiments, since the defibrillator must only connect with the defibrillation accessory device, the defibrillation accessory device may provide a link, potentially including hardware and software, to enable such uses, e.g., by connecting the appropriate pair of electrodes to the defibrillator, or by connecting the appropriate pair of electrodes from one or more multielectrode pads, and/or by providing appropriate display or user interface communications on a display or user interface of a defibrillator to a user, e.g., as may relate to a DSED mode, as described herein.

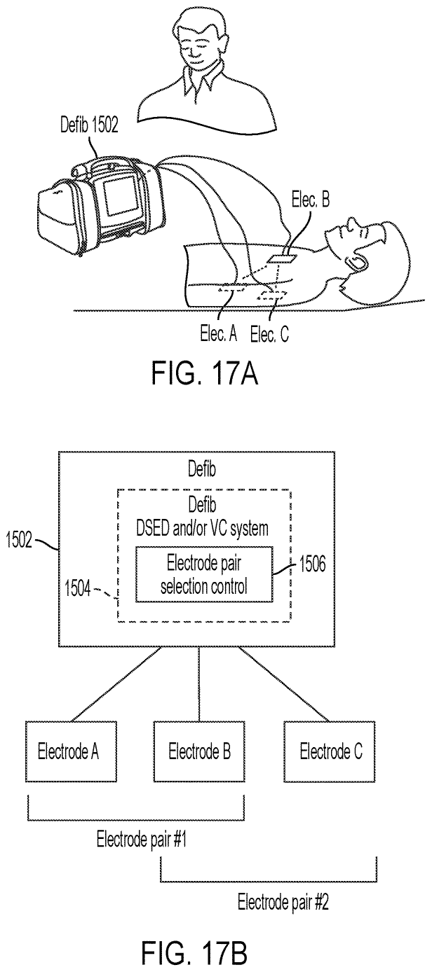

FIGS. 17A and 17B illustrate an example of DSED or defibrillation with vector change including a defibrillator 1502 with a vector change system, and including use of three electrodes, including electrodes A. B and C, as depicted in FIG. 16A-B (although, in other embodiments, the electrodes may be different between the embodiments). In the embodiment shown in FIG. 17A-B, the electrodes A, B and C are connected to the defibrillator 1402, such as via one or more ports.

The defibrillator 1502 includes a defibrillator DSED (or defibrillation with vector change) system 1504, including an electrode pair selection control 1506, which may, for example operate similarly or analogously to the electrode pair selection control 1408 of the defibrillation accessory device 1404 as shown in FIG. 16A-B, such as by causing selection and switching of electrical connection of electrode pairs to the defibrillator 1502. Also similarly to the embodiment shown in FIGS. 16A-B, electrode pairs 1 and 2 are formed using only three electrodes A, B and C, with electrode B being common between both pairs 1 and 2.

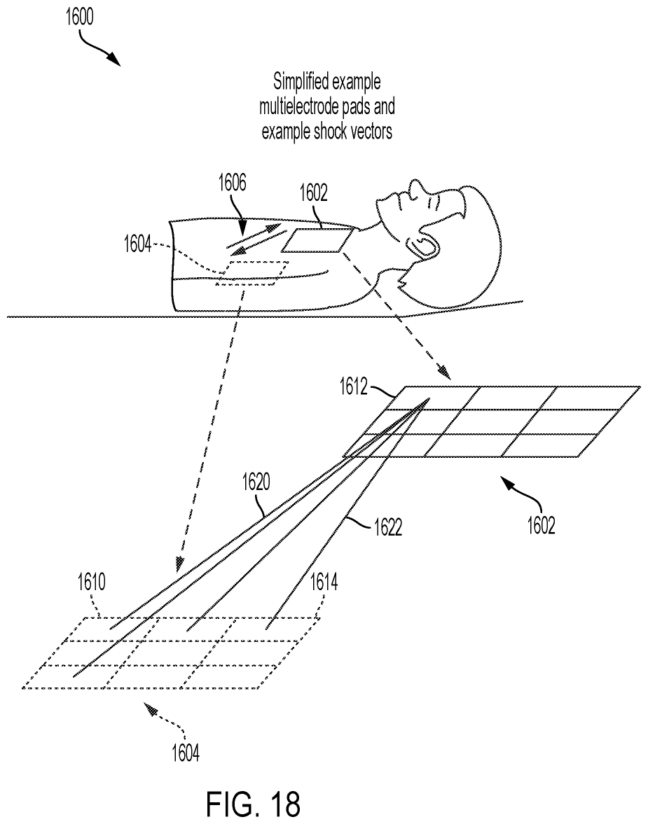

FIG. 18 provides a simplified illustration 1600 of use of two multielectrode pads 1602, 1604, as provided and used in some embodiments, and use thereof, such as in DSED or defibrillation with vector change. As shown, pad 1602 is positioned in an anterior position on the patient, and pad 1604 is positioned on the patient in a position that may include lateral, posterior, or both lateral and posterior positioning areas, for example (however, in other embodiments, other positions of the pads 1602, 1604 are used). Each of the multielectrode pads 1602, 1604 includes (e.g., such as attached with, formed as part of, or incorporated into, the pad 1602, 1604) a network of electrically separated individual electrodes, each shown as a rectangle on each pad 1602, 1604, including, for example, electrode 1612 of pad 1602 and electrodes 1610 and 1614 of electrode pad 1604. In some embodiments, for a particular shock (e.g., of DSED or defibrillation with vector change), any (or some) of the individual electrodes of pad 1602 may be paired with any (or some) of the individual electrodes of pad 1604 to form an electrode pair to be used for a shock, which pair defines the vector line used for the shock. Each of the electrodes of each pad 1602, 1604 may be electrically isolated from each other, and each may be electrically connectable and disconnectable from, e.g., a defibrillator or a defibrillation accessory device. In some embodiments, a defibrillator or accessory device may include an electrode pair control subsystem that selects the pair of electrodes and electrically connects only that pair of electrodes to the defibrillator, or the appropriate defibrillator, for delivery of the corresponding shock using the selected electrode pair and along a vector line defined by selected pair. For a second shock (e.g., of DSED or defibrillation with vector change), the electrode pair control subsystem may select a different pair of electrodes, thereby causing the second shock to be delivered along a different vector line than the first shock.

For example, if electrode 1612 of pad 1602 and electrode 1610 of pad 1604 are selected, then the corresponding shock will be delivered along vector line 1620. However, if electrode 1612 of pad 1602 and electrode 1614 of pad 1604 are selected, then the corresponding shock will be delivered along vector line 1622, which is different than vector line 1620. In some embodiments, any of the electrodes of either of the pads 1602 and 1604 may be paired, allowing a large number of different pairs and vector lines, including different particular vector lines that differ from each other by relatively small directional differences, or differences of varying magnitudes, for example, e.g., as may be deemed or determined to be optimal.

In some embodiments of DSED and defibrillation with vector change, vector lines and pairs of electrodes, e.g., selected for first and second shocks, may be selected in order to optimize, or to attempt to optimize, treatment effect or probably of treatment success, such as may include, e.g., consideration of factors relating to the patient, potentially among other factors. For example, accumulated historical outcome data across many patients may be algorithmically mined to estimate probabilities of success with particular vector lines, vectors, vector sequences and other protocol parameters (e.g., the length of the shock delay period or the shock waveforms).

Additionally, other aspects or parameters of the system or operation may also be so optimized, including, for example, the shock durations, shock waveforms, shock current or voltage levels, whether to use biphasic or monophasic shocks, shock polarities, any shock delay period between shocks, overlapping or simultaneous shocks and associated timing (if/as safe for the patient), the number of shocks used (e.g., 2 or more), etc.

For example, in some embodiments, either or both of various types of physiological and theoretical data, as well as historical treatment data and statistics, may be used in such optimization. For example, in some embodiments, one or more algorithms, such as may include one or more machine learning or artificial intelligence models, may be used in this regard, where input and/or training data may include, e.g., physiological data as well as historical data. The historical data may include, e.g., outcome statistics associated with different vector lines and pairs, and different combinations of first shock and second shock vector lines and pairs, and other system and/or operational factors. This may include, for example, even small differences in, e.g., anterior positioning, lateral positioning and posterior positioning, or other positioning, of electrodes, timing differences, etc.

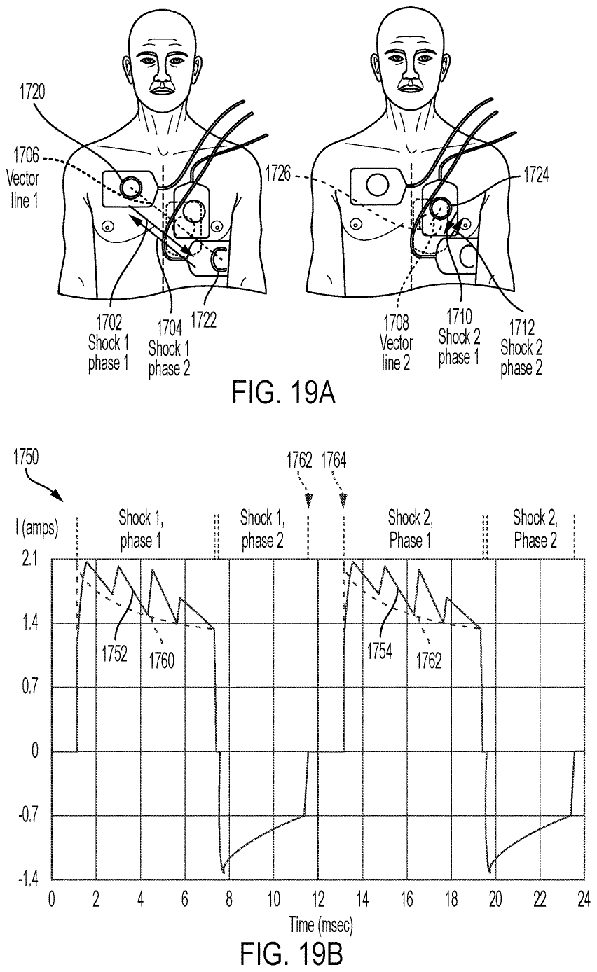

FIGS. 19A and 19B illustrate an example of DSED with no, or only a very small, end-to-start delay between the end of biphasic defibrillation shock 1 and the start of biphasic defibrillation shock 2, each including phase 1 (positive polarity phase) and 2 (negative polarity phase). Specifically, in the example shown, broken line 1762 marks the completion of shock 1 and broken line 1764 marks the start of shock 2, with a less than 1 millisecond end-to-start delay between them.

Specifically, shock 1 is delivered along vector line 1706 using electrode 1720 (positioned in an anterior position) and electrode 1722 (positioned in a lateral position), and shock 2 is delivered along vector line 1708 using electrode 1724 (positioned in an anterior position) and electrode 1726 (positioned in a posterior position). As shown in plot 1750, each of shock 1 phase 1 and shock 2 phase 1 may be delivered in accordance with a biphasic rectilinear current waveform 1752, 1754. In other embodiments, however, any of various different current or voltage waveforms (or others) may be used, including, for example, a truncated exponential waveform, as shown in part by broken lines 1760, 1762 of shock 1 phase 1 and shock 2 phase 1.

Furthermore, in various embodiments, various combinations of biphasic or monophasic shocks may be used in DSED or defibrillation with vector change. This may include, for example, a biphasic shock followed by a monophasic shock or vice versa. It may also include monophasic shocks in different directions along particular vector lines, for example. It may further include three, four, five or more shocks, for example.

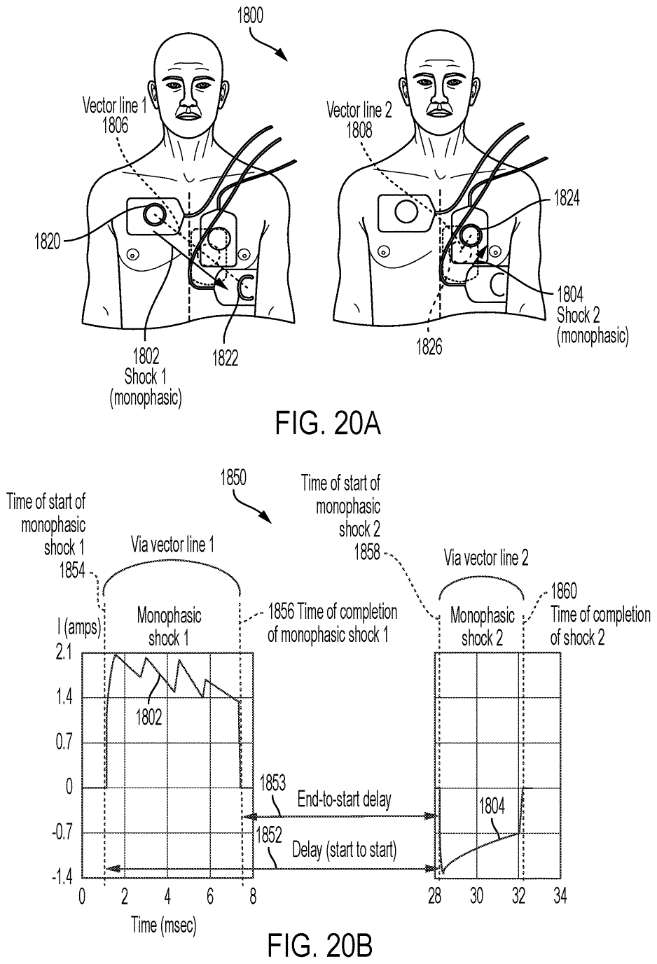

FIGS. 20A and 20B illustrate an example of DSED including use of two monophasic defibrillation shocks. Specifically, monophasic shock 1 is delivered in vector 1802 along vector line 1806 from electrode 1820 (positioned in an anterior position) to electrode 1822 (positioned in a lateral position) and monophasic shock 2 is delivered in vector 1804 along vector line 1808 from electrode 1824 (positioned in an anterior position) to electrode 1826 (positioned in a posterior position).

Plot 1850 shows monophasic shock 1 1802, which is delivered from electrode 1820 to electrode 1822 in vector 1802 along vector line 1 1806, from time of start 1854 to time of completion 1856, and monophasic shock 2 1804, which is delivered in vector 1804 from electrode 1826 to electrode 1824, from time of start 1858 to time of completion 1860. In DSED, the monophasic shocks 1802, 1804 may be separated by a shock delay period of. e.g., 0-75 milliseconds. As depicted, monophasic shock 1 and monophasic shock 2 may similar or identical in waveform to positive and negative phases of a biphasic rectilinear shock, respectively, but may be separated by an end-to-start (end of monophasic shock 1 to start of monophasic shock 2), e.g., 0-75 milliseconds, for example. In the example shown, there is a delay (start of monophasic shock 1 to start of monophasic shock 2) of approximately 27 seconds (from approximately time 1 msec to approximately time 28 msec) and an end-to-start delay of approximately 21 seconds (from approximately time 7.5 msecs to approximately time 28 msecs). In other embodiments, however, each of the monophasic shocks may be of various different durations and waveform types and shapes, for example.

In other embodiments, DSED using biphasic and monophasic shocks of various timing relationships may be used, including simultaneous, overlapping or partially overlapping (to various degrees or for various amounts of time) shock delivery periods (if/as safe for the patient and deemed effective).

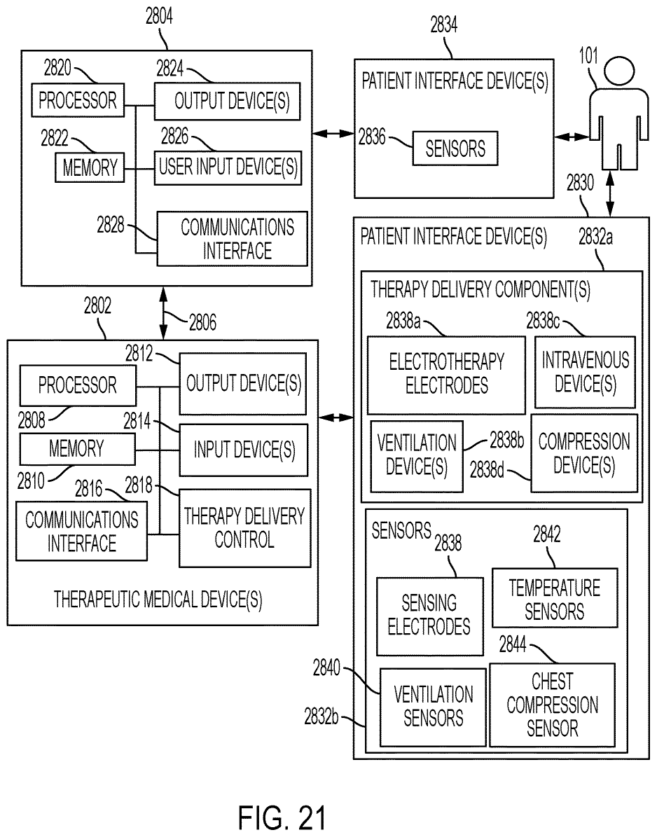

FIG. 21 illustrates an example of components of various devices and systems described with reference to prior figures, such as devices or systems for delivery of multiple defibrillation shocks, for DSED, or for defibrillation with vector change, such as defibrillators, defibrillation accessory devices, and associated systems or devices, as well as other systems and devices. The components 2808, 2810, 2812, 2814, 2816, and 2818 are communicatively coupled (directly and/or indirectly) to each other for bi-directional communication. Similarly, the components 2820, 2822, 2824, 2826, and 2828 are communicatively coupled (directly and/or indirectly) to each other for bi-directional communication.

In some implementations, the components 2808, 2810, 2816, and/or 2818 of the therapeutic medical device 2802 may be combined into one or more discrete components and components 2816 and/or 2818 may be part of the processor 2808. The processor 2808 and the memory 2810 may include and/or be coupled to associated circuitry in order to perform the functions described herein. Additionally, the components 2820, 2822, and 2828 of companion device 2804 may be combined into one or more discrete components and component 2828 may be part of the processor 2820. The processor 2820 and the memory 2822 may include and/or be coupled to associated circuitry in order to perform the functions described herein.

In some implementations, the therapeutic medical device(s) 2802 may include the therapy delivery control module 2818. For example, the therapy delivery control module 2818 may be an electrotherapy delivery circuit that includes one or more high-voltage capacitors configured to store electrical energy for a pacing pulse or one or more defibrillation shocks. The electrotherapy delivery circuit may further include resistors, additional capacitors, relays and/or switches, electrical bridges such as an H-bridge (e.g., including a plurality of insulated gate bipolar transistors or IGBTs), voltage measuring components, and/or current measuring components. As another example, the therapy delivery control module 2818 may be a compression device electro-mechanical controller configured to control a mechanical compression device. As a further example, the therapy delivery control module 2818 may be an electro-mechanical controller configured to control drug delivery, temperature management, ventilation, and/or other type of therapy delivery.

The therapeutic medical device 2802 may incorporate and/or be configured to couple to one or more patient interface devices 2830 and patient interface devices (e.g., of or coupled with a ventilation system such as a BVM ventilation system) that may be coupled with a patient 2849. The patient interface devices 2830 may include one or more therapy delivery component(s) 2832a and one or more sensor(s) 2832b (some of which may be included as part of, e.g., a sensing device according to various embodiments described herein). Similarly, the companion device 2804 may be adapted for medical use and may incorporate and/or be configured to couple to one or more patient interface device(s) 2834. The patient interface device(s) 2834 may include one or more sensors 2836. The sensor(s) 2836 may be substantially as described herein with regard to the sensor(s) 2832b.

The sensor(s) 2832b and 2836 may include sensing electrodes (e.g., the sensing electrodes 2838), ventilation and/or respiration sensors (e.g., the ventilation and/or respiration sensors 2830), temperature sensors (e.g., the temperature sensor 2842), chest compression sensors (e.g., the chest compression sensor 2844), etc. In some implementations, the information obtained from the sensors 2832b and 2836 can be used to generate information displayed at the therapeutic medical device 2802 and simultaneously at the display views at companion device 2804 and described above. In one example, the sensing electrodes 2838 may include cardiac sensing electrodes. The cardiac sensing electrodes may be conductive and/or capacitive electrodes configured to measure changes in a patient's electrophysiology to measure the patient's ECG information. The sensing electrodes 2838 may further measure the transthoracic impedance and/or a heart rate of the patient. The ventilation and/or respiration sensors 2830 may include spirometry sensors, flow sensors, pressure sensors, oxygen and/or carbon dioxide sensors such as, for example, one or more of pulse oximetry sensors, oxygenation sensors (e.g., muscle oxygenation/pH), 02 gas sensors and capnography sensors, impedance sensors, and combinations thereof. The temperature sensors 2842 may include an infrared thermometer, a contact thermometer, a remote thermometer, a liquid crystal thermometer, a thermocouple, a thermistor, etc. and may measure patient temperature internally and/or externally. The chest compression sensor 2844 may include one or more motion sensors including, for example, one or more accelerometers, one or more force sensors, one or more magnetic sensors, one or more velocity sensors, one or more displacement sensors, etc.

The chest compression sensor 2844 may provide one or more signals indicative of the chest motion to the therapeutic medical device 2802 via a wired and/or wireless connection. The chest compression sensor 2844 may be, for example, but not limited to, a compression puck, a smart-phone, a hand-held device, a wearable device, etc. The chest compression sensor 2844 may be configured to detect chest motion imparted by a rescuer and/or an automated chest compression device (e.g., a belt system, a piston system, etc.). The chest compression sensor 2844 may provide signals indicative of chest compression data including displacement data, velocity data, release velocity data, acceleration data, force data, compression rate data, dwell time data, hold time data, blood flow data, blood pressure data, etc. In an implementation, the defibrillation and/or pacing electrodes may include or be configured to couple to the chest compression sensor 2844.

In various implementations, the sensors 2832b and 2836 may include one or more sensor devices configured to provide sensor data that includes, for example, but not limited to ECG, blood pressure, heart rate, respiration rate, heart sounds, lung sounds, respiration sounds, end tidal CO₂, saturation of muscle oxygen (SMO₂), oxygen saturation (e.g., SpO₂ and/or PaO₂), cerebral blood flow, point of care laboratory measurements (e.g., lactate, glucose, etc.), temperature, electroencephalogram (EEG) signals, brain oxygen level, tissue pH, tissue fluid levels, images and/or videos via ultrasound, laryngoscopy, and/or other medical imaging techniques, near-infrared spectroscopy, pneumography, cardiography, and/or patient movement. Images and/or videos may be two-dimensional or three-dimensional, such a various forms of ultrasound imaging.

The one or more therapy delivery components 2832a may include electrotherapy electrodes (e.g., the electrotherapy electrodes 2838a), ventilation device(s) (e.g., the ventilation devices 2838b), intravenous device(s) (e.g., the intravenous devices 2838c), compression device(s) (e.g., the compression devices 2838d), etc. For example, the electrotherapy electrodes 2838a may include defibrillation electrodes, pacing electrodes, and combinations thereof. The ventilation devices 2838b may include a tube, a mask, an abdominal and/or chest compressor (e.g., a belt, a cuirass, etc.), etc. and combinations thereof. The intravenous devices 2838c may include drug delivery devices, fluid delivery devices, and combinations thereof. The compression devices 2838d may include mechanical compression devices such as abdominal compressors, chest compressors, belts, pistons, and combinations thereof. In various implementation, the therapy delivery component(s) 2832a may be configured to provide sensor data and/or be coupled to and/or incorporate sensors. For example, the electrotherapy electrodes 2838a may provide sensor data such as transthoracic impedance, ECG, heart rate, etc. Further the electrotherapy electrodes 2838a may include and or be coupled to a chest compression sensor. As another example, the ventilation devices 2838b may be coupled to and/or incorporate flow sensors, gas species sensors (e.g., oxygen sensor, carbon dioxide sensor, etc.), etc. As a further example, the intravenous devices 2838c may be coupled to and/or incorporate temperature sensors, flow sensors, blood pressure sensors, etc. As yet another example, the compression devices 2838d may be coupled to and/or incorporate chest compression sensors, patient position sensors, etc. The therapy delivery control modules 2818 may be configured to couple to and control the therapy delivery component(s) 2832a, respectively.

The one or more sensor(s) 2832b and 2836 and/or the therapy delivery component(s) 2832a may provide sensor data. The patient data provided at the display screens of the therapeutic medical device 2802 and companion device 2804 may display the sensor data. For example, the therapeutic medical device 2802 may process signals received from the sensor(s) 2832b and/or the therapy delivery component(s) 2832a to determine the sensor data. Similarly, the companion device 2804 may process signals received from the sensor(s) 2836 and/or sensor data from the sensors 2832b received via the therapeutic medical device 2802 to determine the sensor data.

Systems, apparatus and methods are presented herein that include generating and delivering one or more electrotherapeutic pulses according to a specified current waveform to a patient, such as for defibrillation or pacing. It is to be understood that, with reference to diagrams herein that depict circuit components, for instance, additional circuit components beyond those that are depicted may be included. An “electrical circuit” includes one or more electrical components. A “resonant electrical circuit” is a type of electrical circuit that includes inductance and capacitance. A “resonant tank” is a type of resonant electrical circuit.

The description set forth herein in connection with the appended drawings is intended to be a description of various, illustrative embodiments of the disclosed subject matter. Specific features and functionalities are described in connection with each illustrative embodiment; however, it will be apparent to those skilled in the art that the disclosed embodiments may be practiced without each of those specific features and functionalities.

Reference throughout the specification to “one embodiment” or “an embodiment” means that a particular feature, structure, or characteristic described in connection with an embodiment is included in at least one embodiment of the subject matter disclosed. Thus, the appearance of the phrases “in one embodiment” or “in an embodiment” in various places throughout the specification is not necessarily referring to the same embodiment. Further, the particular features, structures or characteristics may be combined in any suitable manner in one or more embodiments. Further, it is intended that embodiments of the disclosed subject matter cover modifications and variations thereof.

As used in the specification and the appended claims, the singular forms “a,” “an,” and “the” include plural referents unless the context expressly dictates otherwise. That is, unless expressly specified otherwise, as used herein the words “a,” “an,” “the,” and the like carry the meaning of “one or more.” Additionally, it is to be understood that terms such as “left,” “right,” “top,” “bottom,” “front,” “rear,” “side,” “height,” “length,” “width,” “upper.” “lower,” “interior,” “exterior,” “inner,” “outer.” and the like that may be used herein merely describe points of reference and do not necessarily limit embodiments of the present disclosure to any particular orientation or configuration. Furthermore, terms such as “first,” “second,” “third,” etc., merely identify one of a number of portions, components, steps, operations, functions, and/or points of reference as disclosed herein, and likewise do not necessarily limit embodiments of the present disclosure to any particular configuration or orientation.

Furthermore, the terms “approximately,” “substantially”, “about,” “proximate,” “minor variation,” and similar terms generally refer to ranges that include the identified value within a margin of 20%, 10%, 5%, or less than 5%, and any values therebetween.

All of the functionalities described in connection with one embodiment are intended to be applicable to the additional embodiments described below except where expressly stated or where the feature or function is incompatible with the additional embodiments. For example, where a given feature or function is expressly described in connection with one embodiment but not expressly mentioned in connection with an alternative embodiment, it should be understood that the inventors intend that that feature or function may be deployed, utilized or implemented in connection with the alternative embodiment unless the feature or function is incompatible with the alternative embodiment.

In some instances, variations of a term may be utilized that may refer to the same or similar concepts, and certain terms may have meanings that are informed by a particular context. Generally, sending, receiving, or transmitting of data may include by wired and/or wireless connection, and/or within one or more wired or wireless networks. Furthermore, sending from a first entity to a second entity, or to be received by the second entity, can include sending from the first entity to the second entity, or to be received by the second entity, directly from the first entity to the second entity, or indirectly via one or more intermediary entities.

Herein, the term adjusting can include changing as well as not changing or maintaining without change, as may be appropriate. Herein, a determined parameter value can include a determined estimated or determined approximated value for the parameter. Herein, the term optimizing may include, for example, improvement or improved operation in one or more aspects, for example, relative to an actual, potential or hypothetical less optimized situation or less optimized operation. Herein, the term adjusting can include changing as well as not changing or maintaining without change, as may be appropriate. Herein, a determined parameter value can include a determined estimated or determined approximated value for the parameter.

Herein, the term monitoring can refer to or include, for example, monitoring or tracking performed by a computerized device utilizing one or more algorithms and not by a person or user, or monitoring by a person or user, or both. Herein, the term continuous can include, among other things, on a periodic basis (with identical or different periods), on a frequent basis, on a repeated basis, or cyclically, for example. Herein, a determined parameter value can include a determined estimated or determined approximated value for the parameter, and the term parameter can refer to a parameter value, for example (e.g., voltage or voltage value). Herein, the term continuous can include, among other things, on a periodic basis (with identical or different periods), on a frequent basis, on a repeated basis, or cyclically, for example.

An alert or alarm, as used herein, may be presented for the attention of a user, such as by being visually or audibly presented, such as via a display, graphical user interface (GUI) or speaker of a device. However, an alert or alarm may also include reference to alert or alarm conditions that are algorithmically identified, recognized or determined by a computerized device and not necessarily presented or displayed. Herein, the term optimizing may include, for example, improvement or improved operation in one or more aspects, for example, relative to an actual, potential or hypothetical less optimized situation or less optimized operation.

The term closed loop control, as used herein, may refer to control of one or more ventilation related or patient related parameters, such as with relatively little or no required user action, participation or intervention, and can include reference to, but is not limited to reference to, fully automated or fully automatically regulated control. Closed loop control may include, for example, device facilitated or algorithmically facilitated tracking, control and adjustment of one or more parameters, which may or may not include user involvement or participation. Where user involvement or participation is included, it may include, for example, confirming a suggested or recommended ventilation setting change or configuration, deciding on implementing a course of action, selecting one of several suggested courses of action, responding to a presented alert or alarm, or other decisions, choices or actions. User involvement or participation could also include, for example, setting or changing a parameter, where a closed loop control algorithm proceeds from there, initially according to the user-set or user-changed parameter setting. In various embodiments, if there is user involvement, it may be, for example, among other things, in whole or in part user-initiated, or in whole or in part prompted, suggested, recommended or required.

While certain embodiments have been described, these embodiments have been presented by way of example only and are not intended to limit the scope of the present disclosures. Indeed, the novel methods, apparatuses and systems described herein can be embodied in a variety of other forms; furthermore, various omissions, substitutions and changes in the form of the methods, apparatuses and systems described herein can be made without departing from the spirit of the present disclosures. The accompanying claims and their equivalents are intended to cover such forms or modifications as would fall within the scope and spirit of the present disclosures.

1. A system for double defibrillation, comprising:
a first defibrillator, comprising a first accessory device connection port, configured to deliver a first defibrillation shock to a patient;
a second defibrillator, comprising a second accessory device connection port, configured to deliver a second defibrillation shock to the patient; and
a defibrillation accessory device, separate from the first defibrillator and the second defibrillator, comprising:
a first defibrillator connection port and a second defibrillator connection port, and
at least one electrode port configured for connection of at least three electrodes comprising at least two electrode pairs comprising a first pair of electrodes defining a first shock vector path and a second pair of electrodes defining a second shock vector path, wherein the first shock vector path is different than the second shock vector path, and wherein delivery of the second defibrillation shock occurs after a shock delay period following delivery of the first defibrillation shock,
wherein the first defibrillator is configured to deliver the first defibrillation shock to the patient via the first pair of electrodes along the first shock vector path, and wherein the second defibrillator is configured to deliver the second defibrillation shock to the patient via the second pair of electrodes along the second shock vector path, and
wherein electrical current for the first defibrillation shock passes through the defibrillation accessory device before reaching the patient, and wherein electrical current for the second defibrillation shock passes through the defibrillation accessory device before reaching the patient.

2-5. (canceled)

6. The system of claim 1,
wherein the first defibrillator and the defibrillation accessory device are configured for cable connection via the first accessory device connection port and the first defibrillator connection port, and wherein the second defibrillator and the defibrillation accessory device are configured for cable connection via the second accessory device connection port and the second defibrillator connection port, and
wherein the first pair of electrodes defines the first shock vector path between a first electrode of the first pair of electrodes and a second electrode of the first pair of electrodes, wherein the second pair of electrodes defines the second shock vector path between a first electrode of the second pair of electrodes and a second electrode of the second pair of electrodes.

7-11. (canceled)

12. The system of claim 1 wherein the defibrillation accessory device is configured to trigger the delivery of the second defibrillation shock to occur after the shock delay period, wherein the triggering comprises at least one current sensing circuit component configured for use in detection of electrical current of the first defibrillation shock, and wherein the at least one current sensing circuit component comprises a transformer.

13. (canceled)

14. The system of claim 1, wherein the defibrillation accessory device is configured to:
determine a first patient transthoracic impedance value associated with determining at least one electrical parameter for the first defibrillation shock, comprising sending electrical current along the first shock vector path and through the patient, and
determine a second patient transthoracic impedance value associated with determining at least one electrical parameter for the second defibrillation shock, comprising sending electrical current along the second shock vector path and through the patient,
wherein determining the first patient transthoracic impedance value and the second patient transthoracic impedance value comprises alternating between (1) the sending of electrical current along the first shock vector path and through the patient and (2) the sending of electrical current along the second shock vector path and through the patient,
send first electrical signaling associated with the first patient transthoracic impedance value to the first defibrillator for use in determining the at least one electrical parameter for the first defibrillation shock, and
send second electrical signaling associated with the second patient transthoracic impedance value to the second defibrillator for use in determining the at least one electrical parameter for the second defibrillation shock.

15. (canceled)

16. The system of claim 1 wherein the defibrillation accessory device is configured to electrically prevent patient transthoracic impedance monitoring by the first defibrillator and the second defibrillator.

17. The system of claim 16, wherein electrically preventing patient transthoracic impedance monitoring by the first defibrillator and the second defibrillator comprises electrically blocking high frequency patient transthoracic impedance monitoring.

18. The system of claim 17, wherein electrically preventing the patient transthoracic impedance monitoring by the first defibrillator and the second defibrillator comprises electrically blocking the high frequency patient transthoracic impedance monitoring while permitting low frequency patient electrocardiogram (ECG) monitoring by at least one of: the first defibrillator and the second defibrillator.

19. The system of claim 16, wherein electrically preventing the patient transthoracic impedance monitoring by the first defibrillator and the second defibrillator comprises electrically blocking high frequency patient transthoracic impedance monitoring and electrically blocking low frequency patient ECG monitoring by at least one of: the first defibrillator and the second defibrillator.

20. The system of claim 19, wherein electrically preventing the patient transthoracic impedance monitoring by the first defibrillator comprises use of at least one first gas discharge tube, and wherein electrically preventing the patient transthoracic impedance monitoring by the second defibrillator comprises use of at least one second gas discharge tube.

21. The system of claim 20, wherein the defibrillation accessory device comprises an electrical circuit to allow patient ECG monitoring by the first defibrillator, wherein electrical current along the electrical circuit does not pass through the at least one first gas discharge tube or the at least one second gas discharge tube.

22. The system of claim 14, wherein:
causing the sending of the first electrical signaling comprises:
determining a first resistance value associated with the determined first patient transthoracic impedance value; and
selecting a first electrical circuit configuration, for a first programmable resistor circuit, associated with the determined first resistance value; and
wherein causing the sending of the second electrical signaling comprises:
determining a second resistance value associated with the determined second patient transthoracic impedance value; and
selecting a second electrical circuit configuration, for a second programmable resistor circuit, to reflect the determined second resistance value.

23. The system of claim 22, wherein selecting the first electrical circuit configuration comprises determining a configuration of at least one switch of the first programmable resistor circuit as open or closed, and wherein selecting the second electrical circuit configuration comprises determining a configuration of at least one switch of the second programmable resistor circuit as open or closed.

24. The system of claim 23, wherein the first resistance value is selected from a set of available resistance values, wherein the first resistance value is closest, among the set of available resistance values, to the determined first patient transthoracic impedance value, and wherein the second resistance value is selected from the set of available resistance values, wherein the second resistance value is closest, among the set of available resistance values, to the determined second patient transthoracic impedance value, wherein the first resistance value is determined so as to be approximately equal to the first patient transthoracic impedance value, and wherein the second resistance value is determined so as to be approximately equal to the second patient transthoracic impedance value, and wherein the first programmable resistor circuit and the second programmable resistor circuit are PWM based.

25-26. (canceled)

27. The system of claim 22, comprising:
a first voltage regulator circuit, electrically connected between the first defibrillator and the first programmable resistor circuit, configured to protect at least the first programmable resistor circuit from over-voltage during delivery of the first defibrillation shock; and
a second voltage regulator circuit, electrically connected between the second defibrillator and the second programmable resistor circuit, configured to protect at least the second programmable resistor circuit from over-voltage during delivery of the second defibrillation shock.

28. The system of claim 27, wherein each of the first voltage regulator circuit and the second voltage regulator circuit comprises at least one cascode circuit.

29. The system of claim 28, wherein:
the first voltage regulator circuit comprises:
a first cascode circuit configured to protect at least the first programmable resistor circuit from overvoltage during a positive phase of the first defibrillation shock, and
a second cascode circuit configured to protect at least the first programmable resistor circuit from overvoltage during a negative phase of the first defibrillation shock, and
the second voltage regulator circuit comprises:
a third cascode circuit configured to protect at least the second programmable resistor circuit from overvoltage during a positive phase of the second defibrillation shock, and
a fourth cascode circuit configured to protect at least the second programmable resistor circuit from overvoltage during a negative phase of the second defibrillation shock.

30. The system of claim 29, wherein:
the first cascode circuit comprises a first high voltage transistor and a first low voltage transistor,
the second cascode circuit comprises a second high voltage transistor and a second low voltage transistor,
the third cascode circuit comprises a third high voltage transistor and a third low voltage transistor, and
the fourth cascode circuit comprises a fourth high voltage transistor and a fourth low voltage transistor.

31. The system of claim 30, wherein:
the first low voltage transistor is electrically connected between the first high voltage transistor and the first programmable resistor circuit,
the second low voltage transistor is electrically connected between the second high voltage transistor and the first programmable resistor circuit,
the third low voltage transistor is electrically connected between the third high voltage transistor and the second programmable resistor circuit, and
the fourth low voltage transistor is electrically connected between the fourth high voltage transistor and the second programmable resistor circuit.

32. The system of claim 1, wherein the first defibrillator comprises:
an energy storage capacitor for providing electrotherapeutic current to the patient, wherein the first defibrillation shock comprises the electrotherapeutic current;
a therapeutic current control network, electrically coupled to the energy storage capacitor, for controlling the electrotherapeutic current to be delivered to the patient, the therapeutic current control network comprising a resonant electrical circuit and at least one current control switch, wherein energy provided by the energy storage capacitor flows through the resonant electrical circuit; and
a controller electrically coupled to the energy storage capacitor and the therapeutic current control network, wherein the controller is configured to, in connection with the energy provided by the energy storage capacitor that flows through the resonant electrical circuit, control operation of the at least one current control switch of the therapeutic current control network in delivering an electrotherapeutic waveform to the patient to correspond with a specified waveform.

33. (canceled)

34. The system of claim 32, the first defibrillator and the second defibrillator comprises:
a battery; and
a bidirectional charging control network, comprising:
a first circuit connected with the battery,
a second circuit connected with the energy storage capacitor, and
a transformer, connected with the first circuit and the second circuit, for use in storing first energy from the battery and in transferring at least a portion of the stored first energy to the energy storage capacitor, and for use in storing second energy from the energy storage capacitor and in transferring at least a portion of the stored second energy to the battery.

35. The system of claim 1, wherein the first defibrillator comprises:
an energy storage capacitor for providing electrotherapeutic current to the patient, wherein the first defibrillation shock comprises the electrotherapeutic current;
a therapeutic current control network, electrically coupled to the energy storage capacitor, for controlling the electrotherapeutic current to be delivered to the patient, the therapeutic current control network comprising a resonant electrical circuit in and at least one current control switch, wherein energy provided by the energy storage capacitor flows through the resonant electrical circuit,
the therapeutic current control network being configured to be operable in an operation mode of two modes of operation, the two modes comprising:
a first mode in which the resonant electrical circuit is connected with the patient in a parallel configuration, and
a second mode in which the resonant electrical circuit is connected with the patient in a series configuration; and
a controller electrically coupled to the energy storage capacitor and the therapeutic current control network, wherein the controller is configured to, in connection with the energy provided by the energy storage capacitor that flows through the resonant electrical circuit, control operation of the at least one current control switch of the therapeutic current control network in delivering an electrotherapeutic waveform to the patient to correspond with a specified waveform.

36. The system of claim 35, wherein the operation mode is selected based at least in part on maximizing a value of a Q factor, wherein the value of the Q factor represents a measure of peak energy relative to dissipated energy with regard to the resonant electrical circuit, wherein the value of the Q factor is affected by the operation mode and a patient impedance.

37-55. (canceled)

56. A system for double sequential defibrillation, comprising:
at least one defibrillator configured to:
deliver a first defibrillation shock to a patient according to at least one first shock vector along a first shock vector path, the first shock vector path being defined by a first pair of electrodes, and
deliver a second defibrillation shock to the patient according to at least one second shock vector along a second shock vector path, the second shock vector path being defined by a second pair of electrodes, wherein the first shock vector path is different than the second shock vector path, and wherein delivery of the second defibrillation shock occurs after a shock delay period following delivery of the first defibrillation shock,
wherein the system is configured to:
determine a first patient transthoracic impedance value associated with determining at least one electrical parameter for the first defibrillation shock, comprising sending electrical current along the first shock vector path and through the patient, and
determine a second patient transthoracic impedance value associated with determining at least one electrical parameter for the second defibrillation shock, comprising sending electrical current along the second shock vector path and through the patient,
wherein determining the first patient transthoracic impedance value and the second patient transthoracic impedance value comprises alternating between (1) the sending of electrical current along the first shock vector path and through the patient and (2) the sending of electrical current along the second shock vector path and through the patient.

57. The system of claim 56, wherein alternating between (1) the sending of electrical current along the first shock vector path and through the patient and (2) the sending of electrical current along the second shock vector path and through the patient comprises continuously sending electrical current, wherein continuously sending electrical current comprises alternating over time between (1) first periods of time of the sending of electrical current along the first shock vector path and through the patient and (2) second periods of time of the sending of electrical current along the second shock vector path and through the patient, wherein, at any particular time, current is either being sent along the first shock vector path and through the patient or along the second shock vector path and through the patient.

58-65. (canceled)

66. The system of claim 56, wherein the at least one defibrillator comprises a first defibrillator configured to deliver the first defibrillation shock and a second defibrillator configured to deliver the second defibrillation shock.

67. (canceled)

68. The system of claim 56, wherein the at least one defibrillator comprises at least one accessory device connection port, and comprising a defibrillation accessory device, separate from the at least one defibrillator, the defibrillation accessory device comprising:
at least one defibrillator connection port, wherein at least one defibrillator and the defibrillation accessory device are configured for cable connection via the at least one accessory device connection port and the at least one defibrillator connection port,
wherein electrical current for the first defibrillation shock passes through the defibrillation accessory device before reaching the patient, and wherein electrical current for the second defibrillation shock passes through the defibrillation accessory device before reaching the patient.

69-184. (canceled)
